(19) United States
(12) Patent Application Publication
(10) Pub. No.: US 2026/0365626 A1
(43) Pub. Date: Nov. 19, 2026

(54) CRYSTALLINE FORMS OF 5-[(2,4-DINITROPHENOXY)METHYL]-1-METHYL-2-NITRO-1H-IMIDAZOLE

(71) Applicant: Rivus Pharmaceuticals, Inc., Charlottesville, VA (US)

(72) Inventors: Muralikrishna DUVVURI, Chapel Hill, NC (US); Donald Herbert LAMUNYON, Eugene, OR (US); Micah Jeffrey BODNER, Eugene, OR (US)

(21) Appl. No.: 19/131,044

(22) PCT Filed: Nov. 20, 2023
(86) PCT No.: PCT/US2023/080519

§ 371 Date: May 19, 2025

Related U.S. Application Data
(60) Provisional application No. 63/384,478, filed on Nov. 21, 2022.

(51) Int. Cl. C07D 233/91; A61K 31/4168

(52) U.S. Cl. CPC C07D 233/91; A61K 31/4168

(57) ABSTRACT

The present disclosure relates to polymorphic forms of 5-[(2,4-dinitrophenoxy)methyl]-1-methyl-2-nitro-1H-imidazole for treating mitochondria-related disorders or conditions.

### CROSS-REFERENCE TO RELATED APPLICATIONS

This application claims the benefit of U.S. Provisional Application No. 63/384,478, filed on Nov. 21, 2022. The entire contents of the aforementioned application are incorporated herein by reference in their entireties.

### FIELD OF THE INVENTION

The present disclosure relates to polymorphic forms of 5-[(2,4-dinitrophenoxy)methyl]-1-methyl-2-nitro-1H-imidazole for treating mitochondria-related disorders or conditions.

### BACKGROUND

The present disclosure provides polymorphic forms of 5-[(2,4-dinitrophenoxy)methyl]-1-methyl-2-nitro-1H-imidazole for treating a subject afflicted with mitochondria-related disorders or conditions, such as metabolic disorders including obesity, diabetes, or diabetes-associated complications.

Mitochondria control metabolism in individual cells by burning sugars and fats. Mitochondrial uncoupling is a robust and natural process that the body utilizes to generate heat. Heat is generated by the mitochondrion via the uncoupling of respiration (Complexes I-IV) from ATP phosphorylation (Complex V). In fact, 20-40% of the calories consumed go toward the generation of body heat. Mitochondria-related disorders or conditions occur when mitochondria fail to produce enough energy for the body to function properly, affecting almost any part of the body including the cells of the brain, adipose tissue, nerves, muscles, heart, lungs, liver, kidneys, pancreas, eyes, and ears.

5-[(2,4-Dinitrophenoxy)methyl]-1-methyl-2-nitro-1H-imidazole is a novel small molecule uncoupler. It works as a controlled metabolic accelerator (CMA). It is designed to effectively address the root cause of metabolic diseases, the accumulation of fat and sugars in the body. CMAs work to improve cellular metabolism and increase energy expenditure and calorie consumption, reducing the accumulation of fat. Using a new controlled and targeted approach, 5-[(2,4-dinitrophenoxy)methyl]-1-methyl-2-nitro-1H-imidazole can increase mitochondrial proton leak, an ongoing process in the body that dissipates energy, and accounts for 20%-40% of daily calories. 5-[(2,4-Dinitrophenoxy)methyl]-1-methyl-2-nitro-1H-imidazole leverages a mitochondrial uncoupling mechanism to increase substrate utilization.

A primary concern for the manufacture of pharmaceutical compounds is the stability of an active substance. An active substance having a stable crystalline morphology may provide consistent processing parameters and pharmaceutical quality. Unstable active substances may affect the reproducibility of the manufacturing process and thus lead to final formulations that do not meet the high quality and other stringent requirements imposed on formulations of pharmaceutical compositions.

There is thus a continuing need for polymorphic forms of 5-[(2,4-dinitrophenoxy)methyl]-1-methyl-2-nitro-1H-imidazole and manufacturing processes for preparing thereof.

### SUMMARY

In one embodiment, the present disclosure provides a compound of 5-[(2,4-dinitrophenoxy)methyl]-1-methyl-2-nitro-1H-imidazole having the structure:

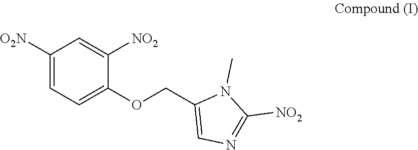 [SMILES: Cn1c(COc2ccc([N+](=O)[O-])cc2[N+](=O)[O-])cnc1[N+](=O)[O-]]

wherein the compound (I) is in a substantially crystalline form.

In another embodiment, the present disclosure provides polymorphic Form A of freebase Compound (I) having an X-ray powder diffraction pattern with characteristic peaks expressed in degrees two-theta at approximately 17.6±0.2, 24.9±0.2, 26.1±0.2, and 30.0±0.2.

In another embodiment, the present disclosure provides polymorphic Form A of freebase Compound (I) having an X-ray powder diffraction pattern with characteristic peaks expressed in degrees two-theta at approximately 16.1±0.2, 16.4±0.2, 17.6±0.2, 17.9±0.2, 24.9±0.2, 26.1±0.2, and 30.0±0.2.

In another embodiment, the present disclosure provides polymorphic Form A of freebase Compound (I) having an X-ray powder diffraction pattern with characteristic peaks expressed in degrees two-theta at approximately 13±0.2, 16.1±0.2, 16.4±0.2, 17.6±0.2, 17.9±0.2, 20.4±0.2, 20.7±0.2, 24.9±0.2, 26.1±0.2, and 30.0±0.2.

In another embodiment, the present disclosure provides polymorphic Form A of freebase Compound (I) having XRPD pattern as shown in FIG. 1.

In another embodiment, the present disclosure provides polymorphic Form B of freebase Compound (I) having an X-ray powder diffraction pattern with characteristic peaks expressed, in degree two-theta, at approximately 8.9±0.2, 13.30±0.2, and 26.2±0.2.

In another embodiment, the present disclosure provides polymorphic Form B of freebase Compound (I) having an X-ray powder diffraction pattern with characteristic peaks expressed, in degree two-theta, at approximately 8.9±0.2, 9.8±0.2, 13.3±0.2, 21.6±0.2, 23.8±0.2, and 26.2±0.2.

In another embodiment, the present disclosure provides polymorphic Form B of freebase Compound (I) having an X-ray powder diffraction pattern with characteristic peaks expressed, in degree two-theta, at approximately 8.9±0.2, 9.8±0.2, 13.3±0.2, 14.0±0.2, 15.7±0.2, 21.6±0.2, 23.8±0.2, 26.2±0.2, 27.3±0.2, and 31.1±0.2.

In certain embodiments, the present disclosure provides polymorphic Form B of freebase Compound (I) having XRPD pattern as shown in FIG. 2.

In some embodiments, the crystalline forms of 5-[(2,4-dinitrophenoxy)methyl]-1-methyl-2-nitro-1H-imidazole are useful for regulating mitochondria activities, reducing adiposity, treating diseases including metabolic disorders, diabetes or diabetes-associated complications such as heart disease and renal failure, and moderating or controlling of weight gain in a subject.

In some embodiments, the disorder is metabolic disorders, diabetes, or diabetes-associated complications, such as heart disease and renal failure, and moderating or controlling of weight gain in a subject.

In some embodiments, the disorder is obesity or excess body fat, diabetes, non-alcoholic fatty liver disease (NAFLD), non-alcoholic steatohepatitis (NASH), hepatic steatosis, insulin resistance or intolerance, dyslipidemia, cardiovascular disease, atherosclerosis.

In some embodiments, the crystalline forms of 5-[(2,4-dinitrophenoxy)methyl]-1-methyl-2-nitro-1H-imidazole are used to reduce adiposity, controlling or preventing of weight gain in a subject, and/or to stimulate oxygen consumption rate (OCR) in a subject, and/or to treat inflammation and fibrosis resulting in NASH in a subject.

### BRIEF DESCRIPTION OF THE DRAWINGS

FIG. 1 shows an X-ray powder diffraction (XRPD) pattern of 5-[(2,4-dinitrophenoxy)methyl]-1-methyl-2-nitro-1H-imidazole Form A.

FIG. 2 shows an X-ray powder diffraction (XRPD) pattern of 5-[(2,4-dinitrophenoxy)methyl]-1-methyl-2-nitro-1H-imidazole Form B.

FIG. 3 shows a differential scanning calorimetry (DSC) profile of 5-[(2,4-dinitrophenoxy)methyl]-1-methyl-2-nitro-1H-imidazole Form A.

FIG. 4 shows a thermal gravimetric analysis (TGA) profile of 5-[(2,4-dinitrophenoxy)methyl]-1-methyl-2-nitro-1H-imidazole Form A.

FIG. 5 shows a ¹H NMR of 5-[(2,4-dinitrophenoxy)methyl]-1-methyl-2-nitro-1H-imidazole Form A.

FIG. 6 shows a differential scanning calorimetry (DSC) profile of 5-[(2,4-dinitrophenoxy)methyl]-1-methyl-2-nitro-1H-imidazole Form B.

FIG. 7 shows a thermal gravimetric analysis (TGA) profile of 5-[(2,4-dinitrophenoxy)methyl]-1-methyl-2-nitro-1H-imidazole Form B.

FIG. 8 shows a ¹HNMR of 5-[(2,4-dinitrophenoxy)methyl]-1-methyl-2-nitro-1H-imidazole Form B.

FIG. 9 shows a ¹³C NMR Spectrum of Form B.

FIG. 10 shows an XRPD patterns overlay of Form A obtained by slow evaporation.

FIG. 11 shows an XRPD patterns overlay of Forms A and B obtained by slow evaporation.

FIG. 12 shows an XRPD patterns overlay of Form A obtained by slow cooling.

FIG. 13 shows an XRPD patterns overlay of Form A obtained by slow cooling.

FIG. 14 shows an XRPD patterns overlay of samples obtained by addition of anti-solvent.

FIG. 15 shows an ortep image of single crystal structure of Form A.

FIG. 16 shows asymmetric unit image of Form A.

FIG. 17 shows a 3D packing image of Form A.

FIG. 18 shows an ortep image of single crystal structure of Form B.

FIG. 19 shows an asymmetric unit image of Form B.

FIG. 20 shows a 3D packing image of Form B.

FIG. 21 shows the plasma concentration of 2,4-dinitrophenol after administration of micronized and non-micronized Compound (I).

FIG. 22A shows mean (±SD) plasma Compound (I) concentration—time plot (linear scale).

FIG. 22B shows mean (±SD) plasma Compound (I) concentration—time plot (semi-log scale).

FIG. 23A shows mean (±SD) plasma 2-4-dinitrophenol concentration—time plot (linear scale).

FIG. 23B shows mean (±SD) plasma 2-4-dinitrophenol concentration—time plot (semi-log scale).

FIG. 24A shows mean (±SD) plasma Compound (I) concentration—time plot (linear scale).

FIG. 24B shows mean (±SD) plasma Compound (I) concentration—time plot (semi-log scale).

FIG. 25A shows mean (±SD) plasma 2-4-dinitrophenol concentration—time plot (linear scale).

FIG. 25B shows mean (±SD) plasma 2-4-dinitrophenol concentration—time plot (semi-log scale).

FIG. 26A compares the plasma Compound (I) concentration following 1050 mg micronized and non-micronized Compound (I) oral administration (Linear Scale).

FIG. 26B compares the plasma Compound (I) concentration following 1050 mg micronized and non-micronized Compound (I) oral administration (semi-log Scale).

FIG. 27A compares the plasma 2,4-dinitrophenol concentration following 1050 mg micronized and non-micronized Compound (I) oral administration (Linear Scale).

FIG. 27B compares the plasma 2,4-dinitrophenol concentration following 1050 mg micronized and non-micronized Compound (I) oral administration (semi-log Scale).

FIG. 28 shows the effect of particle size distribution of Compound (I).

FIG. 29 shows AUC of micronized and non-micronized Compound (I).

FIG. 30A shows simulated dissolution data of non-micronized Compound (I).

FIG. 30B shows simulated dissolution data of micronized Compound (I).

FIG. 30C shows fraction absorbed of non-micronized and micronized Compound (I).

### DETAILED DESCRIPTION

Disclosed herein are crystalline forms of 5-[(2,4-dinitrophenoxy)methyl]-1-methyl-2-nitro-1H-imidazole (Compound (I)). Compound (I) has the following structure:

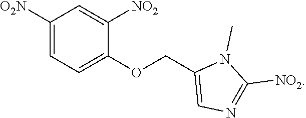 [SMILES: Cn1c(COc2ccc([N+](=O)[O-])cc2[N+](=O)[O-])cnc1[N+](=O)[O-]]

### DEFINITIONS

5-[(2,4-dinitrophenoxy)methyl]-1-methyl-2-nitro-1H-imidazole may be prepared by the procedures described in WO 2018/129258 entitled “Novel Phenyl Derivatives,” published Jul. 12, 2018, and U.S. Pat. No. 10,618,875, entitled “Novel Phenyl Derivatives,” issued Apr. 14, 2020, which are each hereby incorporated by reference in their entireties.

In this disclosure, “Compound 1,” “Compound (I),” “CM1,” “Compound of the invention,” and “Compound of the present invention” are interchangeable. Each refers to 5-[(2,4-dinitrophenoxy)methyl]-1-methyl-2-nitro-1H-imidazole.

Unless defined otherwise, technical and scientific terms used herein have the same meaning as commonly understood by a person of ordinary skill in the art. The following definitions are provided to facilitate understanding of certain terms used frequently herein and are not meant to limit the scope of the present disclosure.

As used herein, an effective amount is defined as the amount required to confer a therapeutic effect on the treated subject, and is typically determined based on age, surface area, weight, and condition of the subject.

As used herein, the term “mammal”, “patient” or “subject” refers to any animal including human, livestock, and companion animals.

As used herein, the term “controlling”, “treating” or “treatment” of a disorder, disease, or condition means (1) decrease, arrest, reduce, inhibit, attenuate, diminish, or stabilize the development of the disease or its clinical symptoms/signs; or (2) cause regression of the disease or its clinical symptoms/signs.

As used herein, “pharmaceutically acceptable” means suitable for use in human, companion animals, and livestock animals.

As used herein, the term “metabolic disorder” refers to a condition characterized by an alteration or disturbance in metabolic function.

As used herein, “crystalline” refers to a solid having a highly regular chemical structure, i.e., having long range structural order in the crystal lattice. The molecules are arranged in a regular, periodic manner in the 3-dimensional space of the lattice. In particular, a crystalline form may be produced as one or more single crystalline forms. For the purposes of this application, the terms “crystalline form”, “single crystalline form,” “crystalline solid form,” “solid form,” and “polymorph” are synonymous and used interchangeably; the terms distinguish between crystals that have different properties (e.g., different XRPD patterns and/or different DSC scan results).

As used herein, the term “substantially crystalline form” refers to at least a particular percentage by weight of Compound (I) are crystalline. Particular weight percentages include at least about 50%, 60%, 70%, 75%, 80%, 85%, 87%, 88%, 89%, 90%, 91%, 92%, 93%, 94%, 95%, 96%, 97%, 98%, 99%, 99.5% and 99.9%.

The term “substantially pure” relates to the composition of a specific crystalline solid form of Compound (I) that may be at least a particular weight percent free of impurities and/or other solid forms of Compound (I). Particular weight percentages are 70%, 75%, 80%, 85%, 90%, 95%, 99%, or any percentage between 70% and 100%. In some embodiments, Compound (I) can be a substantially pure sample of any of the crystalline solid forms described herein, (e.g., Forms A or B). In some embodiments, Compound (I) can be substantially pure Form A. In some embodiments, Compound (I) can be substantially pure Form B.

For the purposes of this application, the terms “Form” and “Pattern” when referring to a specific crystalline form of Compound (I) are used interchangeably. For example, “Form B” and “Pattern B” refer to the same crystalline form of Compound (I).

As used herein, when a crystalline form of a compound is identified using one or more XRPD peaks given as angles two-theta (2θ), each of the 2θ values is understood to mean the given value±0.2 degrees, unless otherwise expressed, for example as the given value±0.3. The term “characteristic peaks” when referring to the peaks in an XRPD pattern of a crystalline form of Compound (I) refers to a collection of certain peaks whose values of 2θ across a range of 0°-40° are, as a whole, uniquely assigned to one of the crystalline forms of Compound (I).

A crystalline form of Compound (I) described herein, e.g., Form A, can melt at a specific temperature or across a range of temperatures. Such a specific temperature or range of temperatures can be represented by the onset temperature (Tₒₙₛₑₜ) of the melting endotherm in the crystalline form's DSC trace. In some embodiments, at such an onset temperature, a sample of a crystalline form of Compound (I) melts and undergoes a concurrently occurring side-process, e.g., recrystallization or chemical decomposition. In some embodiments, at such an onset temperature, a crystalline form of Compound (I) melts in the absence of other concurrently occurring processes.

As used herein, when a crystalline form of a compound is identified using one or more temperatures from a DSC profile (e.g., onset of endothermic transition, melt, etc.), each of the temperature values is understood to mean the given value±2 °C, unless otherwise expressed.

As used herein, the term “anhydrous” or “anhydrate” when referring to a crystalline form of Compound (I) means that no solvent molecules, including those of water, form a portion of the unit cell of the crystalline form. A sample of an anhydrous crystalline form may nonetheless contain solvent molecules that do not form part of the unit cell of the anhydrous crystalline form, e.g., as residual solvent molecule left behind from the production of the crystalline form. In a preferred embodiment, a solvent can make up 0.5% by weight of the total composition of a sample of an anhydrous form. In a more preferred embodiment, a solvent can make up 0.2% by weight of the total composition of a sample of an anhydrous form. In some embodiments, a sample of an anhydrous crystalline form of Compound (I) contains no solvent molecules, e.g., no detectable amount of solvent. The term “solvate” when referring to a crystalline form of Compound (I) means that solvent molecules, e.g., organic solvents and water, form a portion of the unit cell of the crystalline form. Solvates that contain water as the solvent are also referred to herein as “hydrates.” The term “isomorphic” when referring to a crystalline form of Compound (I) means that the form can comprise different chemical constituents, e.g., contain different solvent molecules in the unit cell, but have identical XRPD patterns. Isomorphic crystalline forms are sometimes referred to herein as “isomorphs.”

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material.

“Particles” as used herein are solid forms of Compound (I) having a measurable particle size distribution. The particle size distribution can be calculated by the measuring instrument software and is generally reported in D10, D50, and D90.

The terms D10, D50 and D90 are commonly used to represent the particle size distribution of a given sample. “D10” is the value in which 10% of the particles are equal to or smaller than a defined measurement, for example a particle diameter. “D50” is the value in which 50% of the particles are equal to or smaller than a defined measurement, for example a particle diameter. “D60” is the value in which 60% of the particles are equal to or smaller than a defined measurement, for example a particle diameter. “D70” is the value in which 70% of the particles are equal to or smaller than a defined measurement. “D80” is the value in which 80% of the particles are equal to or smaller than a defined measurement, for example a particle diameter. “D90” is the value in which 90% of the particles are equal to or smaller than a defined measurement, for example a particle diameter.

A “micronized” Compound (I) has been subjected to micronization using any techniques known in the art, including but not limited to, mechanical grinding or shredding, cryogenic grinding, milling, ball milling, wet milling, high pressure homogenization, emulsification and precipitation, precipitation with a compressed fluid anti-solvent, spray freezing into a liquid, rapid expansion from a liquefied-gas solution, evaporative precipitation into an aqueous solution, and air jet micronization.

As used herein, the term “SDD” stands for spray-dried dispersion technology. An SDD is a single-phase, amorphous molecular dispersion of a drug in a polymer matrix. It is a solid solution with the compound molecularly “dissolved” in a solid matrix. As the name suggests, SDDs are obtained by dissolving drug and polymer in an organic solvent and then spray-drying the solution.

### Crystalline Forms of Compound (I)

5-[(2,4-Dinitrophenoxy)methyl]-1-methyl-2-nitro-1H-imidazole (Compound (I)) has the following structure

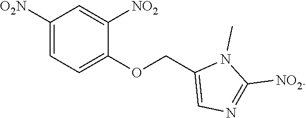 [SMILES: Cn1c(COc2ccc([N+](=O)[O-])cc2[N+](=O)[O-])cnc1[N+](=O)[O-]]

In certain embodiments, the present disclosure provides freebase 5-[(2,4-dinitrophenoxy)methyl]-1-methyl-2-nitro-1H-imidazole Compound (I) in a substantially crystalline form.

In some embodiments, crystalline Compound (I) is polymorphic Form A.

In some embodiments, polymorphic Form A of freebase Compound (I) is characterized by an X-ray powder diffraction pattern having characteristic peaks expressed, in degree 2θ, at approximately 17.6±0.2, 24.9±0.2, 26.0±0.2, and 30.0±0.2.

In some embodiments, polymorphic Form A of freebase Compound (I) is characterized by an X-ray powder diffraction pattern having characteristic peaks expressed, in degree 2θ, at 16.4±0.2, 17.9±0.2, and 20.7±0.2.

In some embodiments, polymorphic Form A of freebase Compound (I) has an X-ray powder diffraction pattern comprising four or more peaks, in 2-theta values, wherein the four or more peaks are selected from 16.4±0.2, 17.6±0.2, 17.9±0.2, 20.7±0.2, 24.9±0.2, 26.0±0.2, and 30.0±0.2.

In some embodiments, polymorphic Form A of freebase Compound (I) is characterized by at least three peaks in an X-ray powder diffraction pattern expressed, in degree 2θ, at approximately 13.0±0.2, 16.1±0.2, 20.4±0.2, and 24.3±0.2.

In some embodiments, polymorphic Form A of freebase Compound (I) has an X-ray powder diffraction pattern comprising six or more peaks, in 2-theta values, wherein the six or more peaks are selected from 13.0±0.2, 16.1±0.2, 16.4±0.2, 17.6±0.2, 17.9±0.2, 20.4 0.2, 20.7±0.2, 24.3±0.2, 24.9±0.2, 26.0±0.2, and 30.0±0.2.

In some embodiments, polymorphic Form A of freebase Compound (I) has an X-ray powder diffraction pattern comprising eight or more peaks, in 2-theta values, wherein the eight or more peaks are selected from 13.0±0.2, 16.1±0.2, 16.4±0.2, 17.6±0.2, 17.9 0.2, 20.4±0.2, 20.7±0.2, 24.3±0.2, 24.9±0.2, 26.0±0.2, and 30.0±0.2.

In some embodiments, polymorphic Form A of freebase Compound (I) is characterized by at least three peaks in an X-ray powder diffraction pattern expressed, in degree 2θ, at approximately 22.8±0.2, 26.2±0.2, 31.1±0.2, and 33.6±0.2.

In some embodiments, polymorphic Form A of freebase Compound (I) has an X-ray powder diffraction pattern comprising ten or more peaks, in 2-theta values, wherein the ten or more peaks are selected from 13.0±0.2, 16.1±0.2, 16.4±0.2, 17.6±0.2, 17.9±0.2, 20.4 0.2, 20.7 0.2, 22.8 0.2, 24.3 0.2, 24.9 0.2, 26.0 0.2, 26.2 0.2, 30.0 0.2, 31.1±0.2, and 33.6±0.2.

In some embodiments, polymorphic Form A of freebase Compound (I) has an X-ray powder diffraction pattern comprising twelve or more peaks, in 2-theta values, wherein the twelve or more peaks are selected from 13.0±0.2, 16.1±0.2, 16.4±0.2, 17.6±0.2, 17.9 0.2, 20.4 0.2, 20.7 0.2, 22.8 0.2, 24.3 0.2, 24.9 0.2, 26.0 0.2, 26.2 0.2, 30.0±0.2, 31.1±0.2, and 33.6±0.2.

In some embodiments, polymorphic Form A of freebase Compound (I) is characterized by at least three peaks in an X-ray powder diffraction pattern expressed, in degree 2θ, at approximately 21.7±0.2, 29.1±0.2, 29.6±0.2, 30.7±0.2, and 37.2±0.2.

In some embodiments, polymorphic Form A of freebase Compound (I) has an X-ray powder diffraction pattern comprising fifteen or more peaks, in 2-theta values, wherein the fifteen or more peaks are selected from 13.0±0.2, 16.1±0.2, 16.4±0.2, 17.6±0.2, 17.9±0.2, 20.4±0.2, 20.7±0.2, 21.7±0.2, 22.8±0.2, 24.3±0.2, 24.9±0.2, 26.0±0.2, 26.2±0.2, 29.1±0.2, 29.6±0.2, 30.0±0.2, 30.7±0.2, 31.1±0.2, 33.6±0.2, and 37.2±0.2.

In some embodiments, polymorphic Form A of freebase Compound (I) has an X-ray powder diffraction pattern comprising eighteen or more peaks, in 2-theta values, wherein the eighteen or more peaks are selected from 13.0±0.2, 16.1±0.2, 16.4±0.2, 17.6±0.2, 17.9±0.2, 20.4±0.2, 20.7±0.2, 21.7±0.2, 22.8±0.2, 24.3±0.2, 24.9±0.2, 26.0±0.2, 26.2±0.2, 29.1±0.2, 29.6±0.2, 30.0±0.2, 30.7±0.2, 31.1±0.2, 33.6±0.2, and 37.2±0.2.

In some embodiments, polymorphic Form A of freebase Compound (I) is characterized by at least three peaks in an X-ray powder diffraction pattern expressed, in degree 2θ, at approximately 16.6±0.2, 24.1±0.2, 25.5±0.2, and 28.8±0.2.

In some embodiments, polymorphic Form A of freebase Compound (I) has an X-ray powder diffraction pattern comprising twenty or more peaks, in 2-theta values, wherein the twenty or more peaks are selected from 13.0±0.2, 16.1±0.2, 16.4±0.2, 16.6±0.2, 17.6 0.2, 17.9±0.2, 20.4±0.2, 20.7±0.2, 21.7±0.2, 22.8±0.2, 24.1±0.2, 24.3±0.2, 24.9±0.2, 25.5±0.2, 26.0±0.2, 26.2±0.2, 28.8±0.2, 29.1±0.2, 29.6±0.2, 30.0±0.2, 30.7±0.2, 31.1±0.2, 33.6±0.2, and 37.2±0.2.

In some embodiments, polymorphic Form A of freebase Compound (I) has an X-ray powder diffraction pattern comprising twenty-two or more peaks, in 2-theta values, wherein the twenty-two or more peaks are selected from 13.0±0.2, 16.1±0.2, 16.4±0.2, 16.6±0.2, 17.6±0.2, 17.9±0.2, 20.4±0.2, 20.7±0.2, 21.7±0.2, 22.8±0.2, 24.1±0.2, 24.3±0.2, 24.9±0.2, 25.5±0.2, 26.0±0.2, 26.2±0.2, 28.8±0.2, 29.1±0.2, 29.6±0.2, 30.0±0.2, 30.7±0.2, 31.1±0.2, 33.6±0.2, and 37.2±0.2.

In some embodiments, polymorphic Form A of freebase Compound (I) is characterized by at least three peaks in an X-ray powder diffraction pattern expressed, in degree 2θ, at approximately 14.4±0.2, 19.0±0.2, 28.5±0.2, 35.7±0.2, 36.2±0.2, and 38.9±0.2.

In some embodiments, polymorphic Form A of freebase Compound (I) has an X-ray powder diffraction pattern comprising twenty-five or more peaks, in 2-theta values, wherein the twenty-five or more peaks are selected from 13.0±0.2, 14.4±0.2, 16.1±0.2, 16.4±0.2, 16.6±0.2, 17.6±0.2, 17.9±0.2, 19.0±0.2, 20.4±0.2, 20.7±0.2, 21.7±0.2, 22.8±0.2, 24.1±0.2, 24.3±0.2, 24.9±0.2, 25.5±0.2, 26.0±0.2, 26.2±0.2, 28.5±0.2, 28.8±0.2, 29.1±0.2, 29.6±0.2, 30.0±0.2, 30.7±0.2, 31.1±0.2, 33.6±0.2, 35.7±0.2, 36.2±0.2, 37.2±0.2, and 38.9±0.2.

In some embodiments, polymorphic Form A of freebase Compound (I) has an X-ray powder diffraction pattern comprising twenty-eight or more peaks, in 2-theta values, wherein the twenty-eight or more peaks are selected from 13.0±0.2, 14.4±0.2, 16.1±0.2, 16.4±0.2, 16.6±0.2, 17.6±0.2, 17.9±0.2, 19.0±0.2, 20.4±0.2, 20.7±0.2, 21.7±0.2, 22.8±0.2, 24.1±0.2, 24.3±0.2, 24.9±0.2, 25.5±0.2, 26.0±0.2, 26.2±0.2, 28.5±0.2, 28.8±0.2, 29.1±0.2, 29.6±0.2, 30.0±0.2, 30.7±0.2, 31.1±0.2, 33.6±0.2, 35.7±0.2, 36.2±0.2, 37.2±0.2, and 38.9±0.2.

In some embodiments, polymorphic Form A of freebase Compound (I) has an X-ray powder diffraction pattern comprising thirty or more peaks, in 2-theta values, wherein the thirty or more peaks are selected from 7.4±0.2, 10.2±0.2, 13.0±0.2, 14.4±0.2, 14.7±0.2, 15.37±0.2, 16.1±0.2, 16.4±0.2, 16.6±0.2, 17.6±0.2, 17.9±0.2, 19.0±0.2, 19.3±0.2, 20.4±0.2, 20.7±0.2, 21.7±0.2, 21.9±0.2, 22.8±0.2, 23.3±0.2, 23.9±0.2, 24.1±0.2, 24.3±0.2, 24.9±0.2, 25.5±0.2, 26.0±0.2, 26.2±0.2, 27.6±0.2, 28.1±0.2, 28.5±0.2, 28.8±0.2, 29.1±0.2, 29.6±0.2, 30.0±0.2, 30.7±0.2, 31.1±0.2, 31.8±0.2, 33.6±0.2, 34.1±0.2, 35.0±0.2, 35.5±0.2, 35.7±0.2, 36.2±0.2, 36.9±0.2, 37.2±0.2, 38.0±0.2, 38.51±0.2, 38.9±0.2, and 39.43±0.2.

In some embodiments, polymorphic Form A of freebase Compound (I) has an X-ray powder diffraction pattern comprising thirty-five or more peaks, in 2-theta values, wherein the thirty-five or more peaks are selected from 7.4±0.2, 10.2±0.2, 13.0±0.2, 14.4±0.2, 14.7±0.2, 15.37±0.2, 16.1±0.2, 16.4±0.2, 16.6±0.2, 17.6±0.2, 17.9±0.2, 19.0±0.2, 19.3±0.2, 20.4±0.2, 20.7±0.2, 21.7±0.2, 21.9±0.2, 22.8±0.2, 23.3±0.2, 23.9±0.2, 24.1±0.2, 24.3±0.2, 24.9±0.2, 25.5±0.2, 26.0±0.2, 26.2±0.2, 27.6±0.2, 28.1±0.2, 28.5±0.2, 28.8±0.2, 29.1±0.2, 29.6±0.2, 30.0±0.2, 30.7±0.2, 31.1±0.2, 31.8±0.2, 33.6±0.2, 34.1±0.2, 35.0±0.2, 35.5±0.2, 35.7±0.2, 36.2±0.2, 36.9±0.2, 37.2±0.2, 38.0±0.2, 38.51±0.2, 38.9±0.2, and 39.43±0.2.

In some embodiments, polymorphic Form A of freebase Compound (I) has an X-ray powder diffraction pattern comprising forty or more peaks, in 2-theta values, wherein the forty or more peaks are selected from 7.4±0.2, 10.2±0.2, 13.0±0.2, 14.4±0.2, 14.7±0.2, 15.37±0.2, 16.1±0.2, 16.4±0.2, 16.6±0.2, 17.6±0.2, 17.9±0.2, 19.0±0.2, 19.3±0.2, 20.4±0.2, 20.7±0.2, 21.7±0.2, 21.9±0.2, 22.8±0.2, 23.3±0.2, 23.9±0.2, 24.1±0.2, 24.3±0.2, 24.9±0.2, 25.5±0.2, 26.0±0.2, 26.2±0.2, 27.6±0.2, 28.1±0.2, 28.5±0.2, 28.8±0.2, 29.1±0.2, 29.6±0.2, 30.0±0.2, 30.7±0.2, 31.1±0.2, 31.8±0.2, 33.6±0.2, 34.1±0.2, 35.0±0.2, 35.5±0.2, 35.7±0.2, 36.2±0.2, 36.9±0.2, 37.2±0.2, 38.0±0.2, 38.51±0.2, 38.9±0.2, and 39.43±0.2.

In some embodiments, polymorphic Form A of freebase Compound (I) is characterized by an X-ray powder diffraction pattern having characteristic peaks expressed, in degree 2θ, at approximately 17.56±0.2, 24.95±0.2, 26.03±0.2, and 29.98±0.2.

In some embodiments, polymorphic Form A of freebase Compound (I) is characterized by an X-ray powder diffraction pattern having characteristic peaks expressed, in degree 2θ, at 16.36±0.2, 17.94±0.2, and 20.70±0.2.

In some embodiments, polymorphic Form A of freebase Compound (I) has an X-ray powder diffraction pattern comprising four or more peaks, in 2-theta values, wherein the four or more peaks are selected from 16.36±0.2, 17.56±0.2, 17.94±0.2, 20.70±0.2, 24.95±0.2, 26.03±0.2, and 29.98±0.2.

In some embodiments, polymorphic Form A of freebase Compound (I) is characterized by at least three peaks in an X-ray powder diffraction pattern expressed, in degree 2θ, at approximately 12.95±0.2, 16.09±0.2, 20.36±0.2, and 24.29±0.2.

In some embodiments, polymorphic Form A of freebase Compound (I) has an X-ray powder diffraction pattern comprising six or more peaks, in 2-theta values, wherein the six or more peaks are selected from 12.95±0.2, 16.09±0.2, 16.36±0.2, 17.56±0.2, 17.94±0.2, 20.36±0.2, 20.70±0.2, 24.29±0.2, 24.95±0.2, 26.03±0.2, and 29.98±0.2.

In some embodiments, polymorphic Form A of freebase Compound (I) has an X-ray powder diffraction pattern comprising eight or more peaks, in 2-theta values, wherein the eight or more peaks are selected from 12.95±0.2, 16.09±0.2, 16.36±0.2, 17.56±0.2, 17.94±0.2, 20.36±0.2, 20.70±0.2, 24.29±0.2, 24.95±0.2, 26.03±0.2, and 29.98±0.2.

In some embodiments, polymorphic Form A of freebase Compound (I) is characterized by at least three peaks in an X-ray powder diffraction pattern expressed, in degree 2θ, at approximately 22.78±0.2, 26.25±0.2, 31.15±0.2, and 33.56±0.2.

In some embodiments, polymorphic Form A of freebase Compound (I) has an X-ray powder diffraction pattern comprising ten or more peaks, in 2-theta values, wherein the ten or more peaks are selected from 12.95±0.2, 16.09±0.2, 16.36±0.2, 17.56±0.2, 17.94±0.2, 20.36±0.2, 20.70±0.2, 22.78±0.2, 24.29±0.2, 24.95±0.2, 26.03±0.2, 26.25±0.2, 29.98±0.2, 31.15±0.2, and 33.56±0.2.

In some embodiments, polymorphic Form A of freebase Compound (I) has an X-ray powder diffraction pattern comprising twelve or more peaks, in 2-theta values, wherein the twelve or more peaks are selected from 12.95±0.2, 16.09±0.2, 16.36±0.2, 17.56±0.2, 17.94±0.2, 20.36±0.2, 20.70±0.2, 22.78±0.2, 24.29±0.2, 24.95±0.2, 26.03±0.2, 26.25±0.2, 29.98±0.2, 31.15±0.2, and 33.56±0.2.

In some embodiments, polymorphic Form A of freebase Compound (I) is characterized by at least three peaks in an X-ray powder diffraction pattern expressed, in degree 2θ, at approximately 21.66±0.2, 29.07±0.2, 29.63±0.2, 30.67±0.2, and 37.25±0.2.

In some embodiments, polymorphic Form A of freebase Compound (I) has an X-ray powder diffraction pattern comprising fifteen or more peaks, in 2-theta values, wherein the fifteen or more peaks are selected from 12.95±0.2, 16.09±0.2, 16.36±0.2, 17.56±0.2, 17.94±0.2, 20.36±0.2, 20.70±0.2, 21.66±0.2, 22.78±0.2, 24.29±0.2, 24.95±0.2, 26.03±0.2, 26.25±0.2, 29.07±0.2, 29.63±0.2, 29.98±0.2, 30.67±0.2, 31.15±0.2, 33.56±0.2, and 37.25±0.2.

In some embodiments, polymorphic Form A of freebase Compound (I) has an X-ray powder diffraction pattern comprising eighteen or more peaks, in 2-theta values, wherein the eighteen or more peaks are selected from 12.95±0.2, 16.09±0.2, 16.36±0.2, 17.56±0.2, 17.94±0.2, 20.36±0.2, 20.70±0.2, 21.66±0.2, 22.78±0.2, 24.29±0.2, 24.95±0.2, 26.03±0.2, 26.25±0.2, 29.07±0.2, 29.63±0.2, 29.98±0.2, 30.67±0.2, 31.15±0.2, 33.56±0.2, and 37.25±0.2.

In some embodiments, polymorphic Form A of freebase Compound (I) is characterized by at least three peaks in an X-ray powder diffraction pattern expressed, in degree 2θ, at approximately 16.63±0.2, 24.11±0.2, 25.49±0.2, and 28.77±0.2.

In some embodiments, polymorphic Form A of freebase Compound (I) has an X-ray powder diffraction pattern comprising twenty or more peaks, in 2-theta values, wherein the twenty or more peaks are selected from 12.95±0.2, 16.09±0.2, 16.36±0.2, 16.63±0.2, 17.56±0.2, 17.94±0.2, 20.36±0.2, 20.70±0.2, 21.66±0.2, 22.78±0.2, 24.11±0.2, 24.29±0.2, 24.95±0.2, 25.49±0.2, 26.03±0.2, 26.25±0.2, 28.77±0.2, 29.07±0.2, 29.63±0.2, 29.98±0.2, 30.67±0.2, 31.15±0.2, 33.56±0.2, and 37.25±0.2.

In some embodiments, polymorphic Form A of freebase Compound (I) has an X-ray powder diffraction pattern comprising twenty-two or more peaks, in 2-theta values, wherein the twenty-two or more peaks are selected from 12.95±0.2, 16.09±0.2, 16.36±0.2, 16.63±0.2, 17.56±0.2, 17.94±0.2, 20.36±0.2, 20.70±0.2, 21.66±0.2, 22.78±0.2, 24.11±0.2, 24.29±0.2, 24.95±0.2, 25.49±0.2, 26.03±0.2, 26.25±0.2, 28.77±0.2, 29.07±0.2, 29.63±0.2, 29.98±0.2, 30.67±0.2, 31.15±0.2, 33.56±0.2, and 37.25±0.2.

In some embodiments, polymorphic Form A of freebase Compound (I) is characterized by at least three peaks in an X-ray powder diffraction pattern expressed, in degree 2θ, at approximately 14.44±0.2, 19.05±0.2, 28.50±0.2, 35.70±0.2, 36.22±0.2, and 38.92±0.2.

In some embodiments, polymorphic Form A of freebase Compound (I) has an X-ray powder diffraction pattern comprising twenty-five or more peaks, in 2-theta values, wherein the twenty-five more peaks are selected from 12.95±0.2, 14.44±0.2, 16.09±0.2, 16.36±0.2, 16.63±0.2, 17.56±0.2, 17.94±0.2, 19.05±0.2, 20.36±0.2, 20.70±0.2, 21.66±0.2, 22.78±0.2, 24.11±0.2, 24.29±0.2, 24.95±0.2, 25.49±0.2, 26.03±0.2, 26.25±0.2, 28.50±0.2, 28.77±0.2, 29.07±0.2, 29.63±0.2, 29.98±0.2, 30.67±0.2, 31.15±0.2, 33.56±0.2, 35.70±0.2, 36.22±0.2, 37.25±0.2, and 38.92±0.2.

In some embodiments, polymorphic Form A of freebase Compound (I) has an X-ray powder diffraction pattern comprising twenty-eight or more peaks, in 2-theta values, wherein the twenty-eight or more peaks are selected from 12.95±0.2, 14.44±0.2, 16.09±0.2, 16.36±0.2, 16.63±0.2, 17.56±0.2, 17.94±0.2, 19.05±0.2, 20.36±0.2, 20.70±0.2, 21.66±0.2, 22.78±0.2, 24.11±0.2, 24.29±0.2, 24.95±0.2, 25.49±0.2, 26.03±0.2, 26.25±0.2, 28.50±0.2, 28.77±0.2, 29.07±0.2, 29.63±0.2, 29.98±0.2, 30.67±0.2, 31.15±0.2, 33.56±0.2, 35.70±0.2, 36.22±0.2, 37.25±0.2, and 38.92±0.2.

In some embodiments, polymorphic Form A of freebase Compound (I) has an X-ray powder diffraction pattern comprising thirty or more peaks, in 2-theta values, wherein the thirty or more peaks are selected from 7.37±0.2, 10.25±0.2, 12.95±0.2, 14.44±0.2, 14.71±0.2, 15.37±0.2, 16.09±0.2, 16.36±0.2, 16.63±0.2, 17.56±0.2, 17.94±0.2, 19.05±0.2, 19.28±0.2, 20.36±0.2, 20.70±0.2, 21.66±0.2, 21.90±0.2, 22.78±0.2, 23.26±0.2, 23.89±0.2, 24.11±0.2, 24.29±0.2, 24.95±0.2, 25.49±0.2, 26.03±0.2, 26.25±0.2, 27.58±0.2, 28.09±0.2, 28.50±0.2, 28.77±0.2, 29.07±0.2, 29.63±0.2, 29.98±0.2, 30.67±0.2, 31.15±0.2, 31.80±0.2, 33.56±0.2, 34.08±0.2, 34.96±0.2, 35.47±0.2, 35.70±0.2, 36.22±0.2, 36.93±0.2, 37.25±0.2, 37.97±0.2, 38.51±0.2, 38.92±0.2, and 39.43±0.2.

In some embodiments, polymorphic Form A of freebase Compound (I) has an X-ray powder diffraction pattern comprising thirty-five or more peaks, in 2-theta values, wherein the thirty-five or more peaks are selected from 7.37±0.2, 10.25±0.2, 12.95±0.2, 14.44±0.2, 14.71±0.2, 15.37±0.2, 16.09±0.2, 16.36±0.2, 16.63±0.2, 17.56±0.2, 17.94±0.2, 19.05±0.2, 19.28±0.2, 20.36±0.2, 20.70±0.2, 21.66±0.2, 21.90±0.2, 22.78±0.2, 23.26±0.2, 23.89±0.2, 24.11±0.2, 24.29±0.2, 24.95±0.2, 25.49±0.2, 26.03±0.2, 26.25±0.2, 27.58±0.2, 28.09±0.2, 28.50±0.2, 28.77±0.2, 29.07±0.2, 29.63±0.2, 29.98±0.2, 30.67±0.2, 31.15±0.2, 31.80±0.2, 33.56±0.2, 34.08±0.2, 34.96±0.2, 35.47±0.2, 35.70±0.2, 36.22±0.2, 36.93±0.2, 37.25±0.2, 37.97±0.2, 38.51±0.2, 38.92±0.2, and 39.43±0.2.

In some embodiments, polymorphic Form A of freebase Compound (I) has an X-ray powder diffraction pattern comprising forty or more peaks, in 2-theta values, wherein the forty or more peaks are selected from 7.37±0.2, 10.25±0.2, 12.95±0.2, 14.44±0.2, 14.71±0.2, 15.37±0.2, 16.09±0.2, 16.36±0.2, 16.63±0.2, 17.56±0.2, 17.94±0.2, 19.05±0.2, 19.28±0.2, 20.36±0.2, 20.70±0.2, 21.66±0.2, 21.90±0.2, 22.78±0.2, 23.26±0.2, 23.89±0.2, 24.11±0.2, 24.29±0.2, 24.95±0.2, 25.49±0.2, 26.03±0.2, 26.25±0.2, 27.58±0.2, 28.09±0.2, 28.50±0.2, 28.77±0.2, 29.07±0.2, 29.63±0.2, 29.98±0.2, 30.67±0.2, 31.15±0.2, 31.80±0.2, 33.56±0.2, 34.08±0.2, 34.96±0.2, 35.47±0.2, 35.70±0.2, 36.22±0.2, 36.93±0.2, 37.25±0.2, 37.97±0.2, 38.51±0.2, 38.92±0.2, and 39.43±0.2.

In some embodiments, polymorphic Form A of freebase Compound (I) is characterized by at least four peaks in an X-ray powder diffraction pattern expressed, in degree 2θ, at approximately 7.3±0.2, 16.0±0.2, 16.3±0.2, and 24.7±0.2.

In some embodiments, polymorphic Form A of freebase Compound (I) characterized by an X-ray powder diffraction pattern having characteristic peaks expressed, in degree 2θ, as shown in Table 1 below.

TABLE 1

| Index | Position | Net Intensity |
|---|---|---|
| 1 | 7.371 | 195.374 |
| 2 | 10.248 | 59.5754 |
| 3 | 12.954 | 1356.69 |
| 4 | 14.439 | 367.742 |
| 5 | 14.714 | 248.948 |
| 6 | 15.370 | 37.5046 |
| 7 | 16.085 | 1807.28 |
| 8 | 16.363 | 2261.94 |
| 9 | 16.626 | 556.079 |
| 10 | 17.555 | 4947.99 |
| 11 | 17.940 | 2195.92 |
| 12 | 19.046 | 322.797 |
| 13 | 19.283 | 274.555 |
| 14 | 20.362 | 1501.25 |
| 15 | 20.699 | 2060.37 |
| 16 | 21.663 | 635.211 |
| 17 | 21.903 | 138.441 |
| 18 | 22.780 | 1166.57 |
| 19 | 23.259 | 267.993 |
| 20 | 23.886 | 78.9929 |
| 21 | 24.106 | 542.343 |
| 22 | 24.291 | 1248.46 |
| 23 | 24.947 | 4840.28 |
| 24 | 25.485 | 452.964 |
| 25 | 26.032 | 9459.21 |
| 26 | 26.249 | 1204.77 |
| 27 | 27.584 | 83.0600 |
| 28 | 28.086 | 110.795 |
| 29 | 28.501 | 393.565 |
| 30 | 28.774 | 543.702 |
| 31 | 29.069 | 755.430 |
| 32 | 29.625 | 734.331 |
| 33 | 29.984 | 6866.13 |
| 34 | 30.667 | 843.452 |
| 35 | 31.149 | 1111.77 |
| 36 | 31.800 | 190.775 |
| 37 | 32.569 | 367.074 |
| 38 | 33.103 | 345.880 |
| 39 | 33.555 | 1011.59 |
| 40 | 34.083 | 173.951 |
| 41 | 34.955 | 60.8173 |
| 42 | 35.465 | 123.869 |
| 43 | 35.696 | 370.413 |
| 44 | 36.215 | 347.181 |
| 45 | 36.934 | 116.601 |
| 46 | 37.245 | 611.594 |
| 47 | 37.967 | 139.086 |
| 48 | 38.513 | 233.270 |
| 49 | 38.920 | 398.744 |
| 50 | 39.426 | 290.024 |

In some embodiments, polymorphic Form A of freebase Compound (I) characterized by an X-ray powder diffraction pattern having characteristic peaks expressed, in degree 2θ, as shown in Table 2 below.

TABLE 2

| Index | Position | Rel. Intensity |
|---|---|---|
| 1 | 7.371 | 2.1% |
| 2 | 10.248 | 0.6% |
| 3 | 12.954 | 14.3% |
| 4 | 14.439 | 3.9% |
| 5 | 14.714 | 2.6% |
| 6 | 15.370 | 0.4% |
| 7 | 16.085 | 19.1% |
| 8 | 16.363 | 23.9% |
| 9 | 16.626 | 5.9% |
| 10 | 17.555 | 52.3% |
| 11 | 17.940 | 23.2% |
| 12 | 19.046 | 3.4% |
| 13 | 19.283 | 2.9% |
| 14 | 20.362 | 15.9% |
| 15 | 20.699 | 21.8% |
| 16 | 21.663 | 6.7% |
| 17 | 21.903 | 1.5% |
| 18 | 22.780 | 12.3% |
| 19 | 23.259 | 2.8% |
| 20 | 23.886 | 0.8% |
| 21 | 24.106 | 5.7% |
| 22 | 24.291 | 13.2% |
| 23 | 24.947 | 51.2% |
| 24 | 25.485 | 4.8% |
| 25 | 26.032 | 100.0% |
| 26 | 26.249 | 12.7% |
| 27 | 27.584 | 0.9% |
| 28 | 28.086 | 1.2% |
| 29 | 28.501 | 4.2% |
| 30 | 28.774 | 5.7% |
| 31 | 29.069 | 8.0% |
| 32 | 29.625 | 7.8% |
| 33 | 29.984 | 72.6% |
| 34 | 30.667 | 8.9% |
| 35 | 31.149 | 11.8% |
| 36 | 31.800 | 2.0% |
| 37 | 32.569 | 3.9% |
| 38 | 33.103 | 3.7% |
| 39 | 33.555 | 10.7% |
| 40 | 34.083 | 1.8% |
| 41 | 34.955 | 0.6% |
| 42 | 35.465 | 1.3% |
| 43 | 35.696 | 3.9% |
| 44 | 36.215 | 3.7% |
| 45 | 36.934 | 1.2% |
| 46 | 37.245 | 6.5% |
| 47 | 37.967 | 1.5% |
| 48 | 38.513 | 2.5% |
| 49 | 38.920 | 4.2% |
| 50 | 39.426 | 3.1% |

In some embodiments, polymorphic Form A of freebase Compound (I) characterized by an X-ray powder diffraction pattern having characteristic peaks expressed, in degree 2θ, as shown in Table 3 below.

TABLE 3

| | | | Net | Gross | Rel. |
|---|---|---|---|---|---|
| Index | Position | d Value | Intensity | Intensity | Intensity |
| 1 | 7.371 | 11.98352 Å | 195.374 | 250.537 | 2.1% |
| 2 | 10.248 | 8.62473 Å | 59.5754 | 127.133 | 0.6% |
| 3 | 12.954 | 6.82875 Å | 1356.69 | 1453.97 | 14.3% |
| 4 | 14.439 | 6.12946 Å | 367.742 | 470.706 | 3.9% |
| 5 | 14.714 | 6.01562 Å | 248.948 | 352.879 | 2.6% |
| 6 | 15.370 | 5.76030 Å | 37.5046 | 151.098 | 0.4% |
| 7 | 16.085 | 5.50572 Å | 1807.28 | 1946.02 | 19.1% |
| 8 | 16.363 | 5.41278 Å | 2261.94 | 2407.47 | 23.9% |
| 9 | 16.626 | 5.32786 Å | 556.079 | 706.464 | 5.9% |
| 10 | 17.555 | 5.04794 Å | 4947.99 | 5103.53 | 52.3% |
| 11 | 17.940 | 4.94057 Å | 2195.92 | 2348.11 | 23.2% |
| 12 | 19.046 | 4.65587 Å | 322.797 | 456.376 | 3.4% |
| 13 | 19.283 | 4.59927 Å | 274.555 | 408.723 | 2.9% |
| 14 | 20.362 | 4.35792 Å | 1501.25 | 1647.19 | 15.9% |
| 15 | 20.699 | 4.28780 Å | 2060.37 | 2208.11 | 21.8% |
| 16 | 21.663 | 4.09904 Å | 635.211 | 790.620 | 6.7% |
| 17 | 21.903 | 4.05460 Å | 138.441 | 300.061 | 1.5% |
| 18 | 22.780 | 3.90059 Å | 1166.57 | 1344.50 | 12.3% |
| 19 | 23.259 | 3.82124 Å | 267.993 | 450.971 | 2.8% |
| 20 | 23.886 | 3.72241 Å | 78.9929 | 273.605 | 0.8% |
| 21 | 24.106 | 3.68895 Å | 542.343 | 744.718 | 5.7% |
| 22 | 24.291 | 3.66114 Å | 1248.46 | 1456.58 | 13.2% |
| 23 | 24.947 | 3.56638 Å | 4840.28 | 5062.66 | 51.2% |
| 24 | 25.485 | 3.49236 Å | 452.964 | 680.055 | 4.8% |
| 25 | 26.032 | 3.42020 Å | 9459.21 | 9684.66 | 100.0% |
| 26 | 26.249 | 3.39244 Å | 1204.77 | 1427.76 | 12.7% |
| 27 | 27.584 | 3.23121 Å | 83.0600 | 271.495 | 0.9% |
| 28 | 28.086 | 3.17448 Å | 110.795 | 311.508 | 1.2% |
| 29 | 28.501 | 3.12923 Å | 393.565 | 610.702 | 4.2% |
| 30 | 28.774 | 3.10013 Å | 543.702 | 769.624 | 5.7% |
| 31 | 29.069 | 3.06937 Å | 755.430 | 988.997 | 8.0% |
| 32 | 29.625 | 3.01299 Å | 734.331 | 978.118 | 7.8% |
| 33 | 29.984 | 2.97772 Å | 6866.13 | 7113.67 | 72.6% |
| 34 | 30.667 | 2.91302 Å | 843.452 | 1090.40 | 8.9% |
| 35 | 31.149 | 2.86901 Å | 1111.77 | 1352.20 | 11.8% |
| 36 | 31.800 | 2.81176 Å | 190.775 | 414.384 | 2.0% |
| 37 | 32.569 | 2.74709 Å | 367.074 | 578.760 | 3.9% |
| 38 | 33.103 | 2.70395 Å | 345.880 | 555.690 | 3.7% |
| 39 | 33.555 | 2.66860 Å | 1011.59 | 1214.99 | 10.7% |
| 40 | 34.083 | 2.62843 Å | 173.951 | 364.212 | 1.8% |
| 41 | 34.955 | 2.56485 Å | 60.8173 | 242.690 | 0.6% |
| 42 | 35.465 | 2.52910 Å | 123.869 | 303.996 | 1.3% |
| 43 | 35.696 | 2.51330 Å | 370.413 | 549.733 | 3.9% |
| 44 | 36.215 | 2.47842 Å | 347.181 | 520.449 | 3.7% |
| 45 | 36.934 | 2.43181 Å | 116.601 | 290.342 | 1.2% |
| 46 | 37.245 | 2.41225 Å | 611.594 | 792.726 | 6.5% |
| 47 | 37.967 | 2.36802 Å | 139.086 | 331.117 | 1.5% |
| 48 | 38.513 | 2.33565 Å | 233.270 | 428.798 | 2.5% |
| 49 | 38.920 | 2.31217 Å | 398.744 | 592.662 | 4.2% |
| 50 | 39.426 | 2.28365 Å | 290.024 | 476.915 | 3.1% |

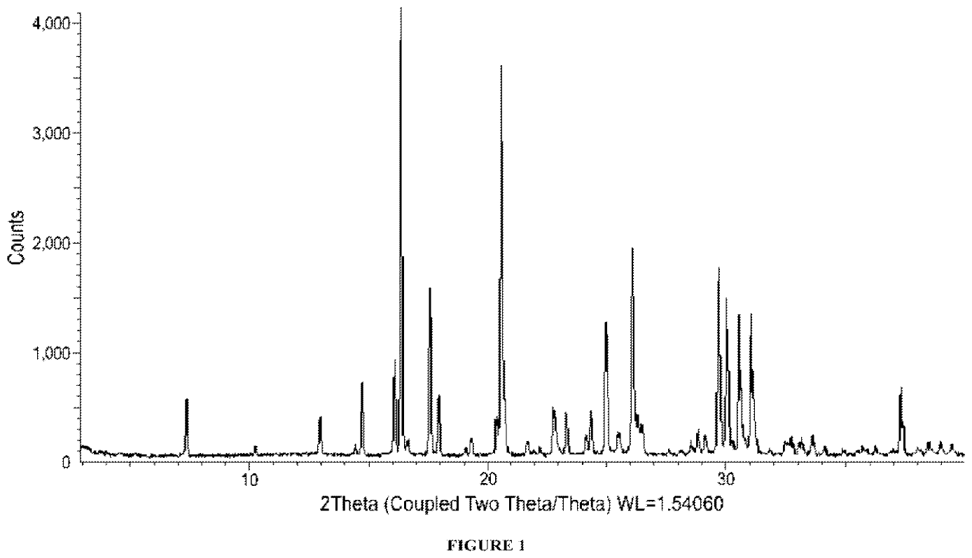

In some embodiments, the present disclosure provides polymorphic Form A of freebase Compound (I) having an XRPD pattern as shown in FIG. 1.

In some embodiments, polymorphic Form A of freebase Compound (I) has a differential scanning calorimetry thermogram (DSC) profile characterized by an endothermic transition at a temperature between 157 °C±3 and 162 °C±3 and a second endothermic transition at 183 °C±3.

In some embodiments, polymorphic Form A of freebase Compound (I) has a thermal gravimetric analysis (TGA) profile characterized by about 0.704% of weight loss at 175 °C±3.

In some embodiments, crystalline Compound (I) is polymorphic Form B.

In some embodiments, polymorphic Form B of freebase Compound (I) is characterized by an X-ray powder diffraction pattern having characteristic peaks expressed, in degree 2θ, at approximately 13.3±0.2, 23.8±0.2, and 26.3±0.2.

In some embodiments, polymorphic Form B of freebase Compound (I) is characterized by an X-ray powder diffraction pattern having characteristic peaks expressed, in degree 2θ, at 9.8±0.2, 21.6±0.2, 27.3±0.2, and 28.1±0.2.

In some embodiments, polymorphic Form B of freebase Compound (I) has an X-ray powder diffraction pattern comprising four or more peaks, in 2-theta values, wherein the four or more peaks are selected from 9.8±0.2, 13.3±0.2, 21.6±0.2, 23.8±0.2, 26.3±0.2, 27.3±0.2, and 28.1±0.2.

In some embodiments, polymorphic Form B of freebase Compound (I) is characterized by an X-ray powder diffraction pattern having characteristic peaks expressed, in degree 2θ, at approximately 14.0±0.2, 25.8±0.2, 26.6±0.2, and 31.1±0.2.

In some embodiments, polymorphic Form B of freebase Compound (I) has an X-ray powder diffraction pattern comprising six or more peaks, in 2-theta values, wherein the six or more peaks are selected from 9.8±0.2, 13.3±0.2, 14.0±0.2, 21.6±0.2, 23.8±10.2, 25.8±0.2, 26.3±0.2, 26.6±0.2, 27.3±0.2, 28.1±0.2, and 31.1±0.2.

In some embodiments, polymorphic Form B of freebase Compound (I) has an X-ray powder diffraction pattern comprising eight or more peaks, in 2-theta values, wherein the eight or more peaks are selected from 9.8±0.2, 13.3±0.2, 14.0±0.2, 21.6±0.2, 23.8±0.2, 25.8±0.2, 26.3±0.2, 26.6±0.2, 27.3±0.2, 28.1±0.2, and 31.1±0.2.

In some embodiments, polymorphic Form B of freebase Compound (I) is characterized by an X-ray powder diffraction pattern having characteristic peaks expressed, in degree 2θ, at 9.0±0.2, 21.2±0.2, 23.2±0.2, 31.8±0.2 and 33.0±0.2.

In some embodiments, polymorphic Form B of freebase Compound (I) has an X-ray powder diffraction pattern comprising ten or more peaks, in 2-theta values, wherein the ten or more peaks are selected from 9.8±0.2, 9.0±0.2, 13.3±0.2, 14.0±0.2, 21.6±0.2, 21.2±0.2, 23.2±0.2, 23.8±0.2, 25.8±0.2, 26.3±0.2, 26.6±0.2, 27.3±0.2, 28.1±0.2, 31.1±0.2, 31.8±0.2, and 33.0±0.2.

In some embodiments, polymorphic Form B of freebase Compound (I) has an X-ray powder diffraction pattern comprising twelve or more peaks, in 2-theta values, wherein the twelve or more peaks are selected from 9.8±0.2, 9.0±0.2, 13.3±0.2, 14.0±0.2, 21.6±0.2, 21.2±0.2, 23.2±0.2, 23.8±0.2, 25.8±0.2, 26.3±0.2, 26.6±0.2, 27.3±0.2, 28.1±0.2, 31.1±0.2, 31.8±0.2, and 33.0±0.2.

In some embodiments, polymorphic Form B of freebase Compound (I) is characterized by an X-ray powder diffraction pattern having characteristic peaks expressed, in degree 2θ, at 17.7±0.2, 19.1±0.2, 30.0±0.2, and 34.7±0.2.

In some embodiments, polymorphic Form B of freebase Compound (I) has an X-ray powder diffraction pattern comprising fourteen or more peaks, in 2-theta values, wherein the fourteen or more peaks are selected from 9.8±0.2, 9.0±0.2, 13.3±0.2, 14.0±0.2, 17.7±0.2, 19.1±0.2, 21.6±0.2, 21.2±0.2, 23.2±0.2, 23.8±0.2, 25.8±0.2, 26.3±0.2, 26.6±0.2, 27.3±0.2, 28.1±0.2, 30.0±0.2, 31.1±0.2, 31.8±0.2, 33.0±0.2, and 34.7±0.2.

In some embodiments, polymorphic Form B of freebase Compound (I) has an X-ray powder diffraction pattern comprising sixteen or more peaks, in 2-theta values, wherein the sixteen or more peaks are selected from 9.8±0.2, 9.0±0.2, 13.3±0.2, 14.0±0.2, 17.7±0.2, 19.1±0.2, 21.6±0.2, 21.2±0.2, 23.2±0.2, 23.8±0.2, 25.8±0.2, 26.3±0.2, 26.6±0.2, 27.3±0.2, 28.1±0.2, 30.0±0.2, 31.1±0.2, 31.8±0.2, 33.0±0.2, and 34.7±0.2.

In some embodiments, polymorphic Form B of freebase Compound (I) is characterized by an X-ray powder diffraction pattern having characteristic peaks expressed, in degree 2θ, at approximately 10.2±0.2, 17.2±0.2, 18.5±0.2, 28.7±0.2, and 35.3±0.2.

In some embodiments, polymorphic Form B of freebase Compound (I) has an X-ray powder diffraction pattern comprising eighteen or more peaks, in 2-theta values, wherein the eighteen or more peaks are selected from 9.8±0.2, 9.0±0.2, 10.2±0.2, 13.3±0.2, 14.0±0.2, 17.2±0.2, 17.7±0.2, 18.5±0.2, 19.1±0.2, 21.6±0.2, 21.2±0.2, 23.2±0.2, 23.8±0.2, 25.8±0.2, 26.3±0.2, 26.6±0.2, 27.3±0.2, 28.1±0.2, 28.7±0.2, 30.0±0.2, 31.1±0.2, 31.8±0.2, 33.0±0.2, 34.7±0.2, and 35.3±0.2.

In some embodiments, polymorphic Form B of freebase Compound (I) has an X-ray powder diffraction pattern comprising twenty or more peaks, in 2-theta values, wherein the twenty or more peaks are selected from 9.8±0.2, 9.0±0.2, 10.2±0.2, 13.3±0.2, 14.0±0.2, 17.2±0.2, 17.7±0.2, 18.5±0.2, 19.1±0.2, 21.6±0.2, 21.2±0.2, 23.2±0.2, 23.8±0.2, 25.8±0.2, 26.3±0.2, 26.6±0.2, 27.3±0.2, 28.1±0.2, 28.7±0.2, 30.0±0.2, 31.1±0.2, 31.8±0.2, 33.0±0.2, 34.7±0.2, and 35.3±0.2.

In some embodiments, polymorphic Form B of freebase Compound (I) has an X-ray powder diffraction pattern comprising twenty-two or more peaks, in 2-theta values, wherein the twenty-two or more peaks are selected from 9.8±0.2, 9.0±0.2, 10.2±0.2, 13.3±0.2, 14.0±0.2, 17.2±0.2, 17.7±0.2, 18.5±0.2, 19.1±0.2, 20.4±0.2, 21.6±0.2, 21.2±0.2, 23.2±0.2, 23.8±0.2, 24.6±0.2, 25.0±0.2, 25.8±0.2, 26.3±0.2, 26.6±0.2, 27.3±0.2, 28.1±0.2, 28.7±0.2, 30.0±0.2, 30.4±0.2, 31.1±0.2, 31.8±0.2, 32.4±0.2, 33.0±0.2, 33.5±0.2, 34.1±0.2, 34.7±0.2, 35.3±0.2, 36.2±0.2, 37.3±0.2, 38.1±0.2, 38.8±0.2.

In some embodiments, polymorphic Form B of freebase Compound (I) has an X-ray powder diffraction pattern comprising twenty-five or more peaks, in 2-theta values, wherein the twenty-five or more peaks are selected from 9.8±0.2, 9.0±0.2, 10.2±0.2, 13.3±0.2, 14.0±0.2, 17.2±0.2, 17.7±0.2, 18.5±0.2, 19.1±0.2, 20.4±0.2, 21.6±0.2, 21.2±0.2, 23.2±0.2, 23.8±0.2, 24.6±0.2, 25.0±0.2, 25.8±0.2, 26.3±0.2, 26.6±0.2, 27.3±0.2, 28.1±0.2, 28.7±0.2, 30.0±0.2, 30.4±0.2, 31.1±0.2, 31.8±0.2, 32.4±0.2, 33.0±0.2, 33.5±0.2, 34.1±0.2, 34.7±0.2, 35.3±0.2, 36.2±0.2, 37.3±0.2, 38.1±0.2, 38.8±0.2.

In some embodiments, polymorphic Form B of freebase Compound (I) has an X-ray powder diffraction pattern comprising thirty or more peaks, in 2-theta values, wherein the thirty or more peaks are selected from 9.8±0.2, 9.0±0.2, 10.2±0.2, 13.3±0.2, 14.0±0.2, 17.2±0.2, 17.7±0.2, 18.5±0.2, 19.1±0.2, 20.4±0.2, 21.6±0.2, 21.2±0.2, 23.2±0.2, 23.8±0.2, 24.6±0.2, 25.0±0.2, 25.8±0.2, 26.3±0.2, 26.6±0.2, 27.3±0.2, 28.1±0.2, 28.7±0.2, 30.0±0.2, 30.4±0.2, 31.1±0.2, 31.8±0.2, 32.4±0.2, 33.0±0.2, 33.5±0.2, 34.1±0.2, 34.7±0.2, 35.3±0.2, 36.2±0.2, 37.3±0.2, 38.1±0.2, 38.8±0.2.

In some embodiments, polymorphic Form B of freebase Compound (I) has an X-ray powder diffraction pattern comprising thirty-four or more peaks, in 2-theta values, wherein the thirty-four or more peaks are selected from 9.8±0.2, 9.0±0.2, 10.2±0.2, 13.3±0.2, 14.0±0.2, 17.2±0.2, 17.7±0.2, 18.5±0.2, 19.1±0.2, 20.4±0.2, 21.6±0.2, 21.2±0.2, 23.2±0.2, 23.8±0.2, 24.6±0.2, 25.0±0.2, 25.8±0.2, 26.3±0.2, 26.6±0.2, 27.3±0.2, 28.1±0.2, 28.7±0.2, 30.0±0.2, 30.4±0.2, 31.1±0.2, 31.8±0.2, 32.4±0.2, 33.0±0.2, 33.5±0.2, 34.1±0.2, 34.7±0.2, 35.3±0.2, 36.2±0.2, 37.3±0.2, 38.1±0.2, 38.8±0.2.

In some embodiments, polymorphic Form B of freebase Compound (I) is characterized by an X-ray powder diffraction pattern having characteristic peaks expressed, in degree 2θ, at approximately 9.8±0.2, 9.0±0.2, 10.2±0.2, 13.3±0.2, 14.0±0.2, 17.2±0.2, 17.7±0.2, 18.5±0.2, 19.1±0.2, 20.4±0.2, 21.6±0.2, 21.2±0.2, 23.2±0.2, 23.8±0.2, 24.6±0.2, 25.0±0.2, 25.8±0.2, 26.3±0.2, 26.6±0.2, 27.3±0.2, 28.1±0.2, 28.7±0.2, 30.0±0.2, 30.4±0.2, 31.1±0.2, 31.8±0.2, 32.4±0.2, 33.0±0.2, 33.5±0.2, 34.1±0.2, 34.7±0.2, 35.3±0.2, 36.2±0.2, 37.3±0.2, 38.1±0.2, 38.8±0.2.

In some embodiments, polymorphic Form B of freebase Compound (I) has an X-ray powder diffraction pattern having characteristic peaks expressed in degrees two-theta at approximately 13.26±0.2, 23.78±0.2, and 26.26±0.2.

In some embodiments, polymorphic Form B of freebase Compound (I) has an X-ray powder diffraction pattern having characteristic peaks expressed in degrees two-theta at approximately 9.81±0.2, 21.58±0.2, 27.27±0.2, and 28.10±0.2

In some embodiments, polymorphic Form B of freebase Compound (I) has an X-ray powder diffraction pattern comprising four or more peaks, in 2-theta values, wherein the four or more peaks are selected from 9.81±0.2, 13.26±0.2, 21.58±0.2, 23.78±0.2, 26.26±0.2, 27.27±0.2, and 28.10±0.2.

In some embodiments, polymorphic Form B of freebase Compound (I) is characterized by an X-ray powder diffraction pattern having characteristic peaks expressed, in degree 2θ, at approximately 13.99±0.2, 25.78±0.2, 26.63±0.2, and 31.08±0.2.

In some embodiments, polymorphic Form B of freebase Compound (I) has an X-ray powder diffraction pattern comprising six or more peaks, in 2-theta values, wherein the six or more peaks are selected from 9.81±0.2, 13.26±0.2, 13.99±0.2, 21.58±0.2, 23.78±0.2, 25.78±0.2, 26.26±0.2, 26.63±0.2, 27.27±0.2, 28.10±0.2, and 31.08±0.2.

In some embodiments, polymorphic Form B of freebase Compound (I) has an X-ray powder diffraction pattern comprising eight or more peaks, in 2-theta values, wherein the eight or more peaks are selected from 9.81±0.2, 13.26±0.2, 13.99±0.2, 21.58±0.2, 23.78±0.2, 25.78±0.2, 26.26±0.2, 26.63±0.2, 27.27±0.2, 28.10±0.2, and 31.08±0.2.

In some embodiments, polymorphic Form B of freebase Compound (I) is characterized by an X-ray powder diffraction pattern having characteristic peaks expressed, in degree 2θ, at 8.95±0.2, 21.23±0.2, 23.20±0.2, 31.77±0.2 and 32.95±0.2.

In some embodiments, polymorphic Form B of freebase Compound (I) has an X-ray powder diffraction pattern comprising ten or more peaks, in 2-theta values, wherein the ten or more peaks are selected from 9.81±0.2, 8.95±0.2, 13.26±0.2, 13.99±0.2, 21.58±0.2, 21.23±0.2, 23.20±0.2, 23.78±0.2, 25.78±0.2, 26.26±0.2, 26.63±0.2, 27.27±0.2, 28.10±0.2, 31.08±0.2, 31.77±0.2, and 32.95±0.2.

In some embodiments, polymorphic Form B of freebase Compound (I) has an X-ray powder diffraction pattern comprising twelve or more peaks, in 2-theta values, wherein the twelve or more peaks are selected from 9.81±0.2, 8.95±0.2, 13.26±0.2, 13.99±0.2, 21.58±0.2, 21.23±0.2, 23.20±0.2, 23.78±0.2, 25.78±0.2, 26.26±0.2, 26.63±0.2, 27.27±0.2, 28.10±0.2, 31.08±0.2, 31.77±0.2, and 32.95±0.2.

In some embodiments, polymorphic Form B of freebase Compound (I) is characterized by an X-ray powder diffraction pattern having characteristic peaks expressed, in degree 2θ, at 17.66±0.2, 19.15±0.2, 30.0±0.2, and 34.74±0.2.

In some embodiments, polymorphic Form B of freebase Compound (I) has an X-ray powder diffraction pattern comprising fourteen or more peaks, in 2-theta values, wherein the fourteen or more peaks are selected from 9.81±0.2, 8.95±0.2, 13.26±0.2, 13.99±0.2, 17.66±0.2, 19.15±0.2, 21.58±0.2, 21.23±0.2, 23.20±0.2, 23.78±0.2, 25.78±0.2, 26.26±0.2, 26.63±0.2, 27.27±0.2, 28.10±0.2, 30.00±0.2, 31.08±0.2, 31.77±0.2, 32.95±0.2, and 34.74±0.2.

In some embodiments, polymorphic Form B of freebase Compound (I) has an X-ray powder diffraction pattern comprising sixteen or more peaks, in 2-theta values, wherein the sixteen or more peaks are selected from 9.81±0.2, 8.95±0.2, 13.26±0.2, 13.99±0.2, 17.66±0.2, 19.15±0.2, 21.58±0.2, 21.23±0.2, 23.20±0.2, 23.78±0.2, 25.78±0.2, 26.26±0.2, 26.63±0.2, 27.27±0.2, 28.10±0.2, 30.00±0.2, 31.08±0.2, 31.77±0.2, 32.95±0.2, and 34.74±0.2.

In some embodiments, polymorphic Form B of freebase Compound (I) is characterized by an X-ray powder diffraction pattern having characteristic peaks expressed, in degree 2θ, at approximately 10.20±0.2, 17.21±0.2, 18.55±0.2, 28.71±0.2, and 35.27±0.2.

In some embodiments, polymorphic Form B of freebase Compound (I) has an X-ray powder diffraction pattern comprising eighteen or more peaks, in 2-theta values, wherein the eighteen or more peaks are selected from 9.81±0.2, 8.95±0.2, 10.20±0.2, 13.26±0.2, 13.99±0.2, 17.21±0.2, 17.66±0.2, 18.55±0.2, 19.15±0.2, 21.58±0.2, 21.23±0.2, 23.20±0.2, 23.78±0.2, 25.78±0.2, 26.26±0.2, 26.63±0.2, 27.27±0.2, 28.10±0.2, 28.71±0.2, 30.00±0.2, 31.08±0.2, 31.77±0.2, 32.95±0.2, 34.74±0.2, and 35.27±0.2.

In some embodiments, polymorphic Form B of freebase Compound (I) has an X-ray powder diffraction pattern comprising twenty or more peaks, in 2-theta values, wherein the twenty or more peaks are selected from 9.81±0.2, 8.95±0.2, 10.20±0.2, 13.26±0.2, 13.99±0.2, 17.21±0.2, 17.66±0.2, 18.55±0.2, 19.15±0.2, 21.58±0.2, 21.23±0.2, 23.20±0.2, 23.78±0.2, 25.78±0.2, 26.26±0.2, 26.63±0.2, 27.27±0.2, 28.10±0.2, 28.71±0.2, 30.00±0.2, 31.08±0.2, 31.77±0.2, 32.95±0.2, 34.74±0.2, and 35.27±0.2.

In some embodiments, polymorphic Form B of freebase Compound (I) has an X-ray powder diffraction pattern comprising twenty-two or more peaks, in 2-theta values, wherein the twenty-two or more peaks are selected from 9.81±0.2, 8.95±0.2, 10.20±0.2, 13.26±0.2, 13.99±0.2, 17.21±0.2, 17.66±0.2, 18.55±0.2, 19.15±0.2, 20.42±0.2, 21.58±0.2, 21.23±0.2, 23.20±0.2, 23.78±0.2, 24.58±0.2, 25.00±0.2, 25.78±0.2, 26.26±0.2, 26.63±0.2, 27.27±0.2, 28.10±0.2, 28.71±0.2, 30.00±0.2, 30.41±0.2, 31.08±0.2, 31.77±0.2, 32.42±0.2, 32.95±0.2, 33.50±0.2, 34.09±0.2, 34.74±0.2, 35.27±0.2, 36.21±0.2, 37.30±0.2, 38.11±0.2, 38.77±0.2.

In some embodiments, polymorphic Form B of freebase Compound (I) has an X-ray powder diffraction pattern comprising twenty-five or more peaks, in 2-theta values, wherein the twenty-five or more peaks are selected from 9.81±0.2, 8.95±0.2, 10.20±0.2, 13.26±0.2, 13.99±0.2, 17.21±0.2, 17.66±0.2, 18.55±0.2, 19.15±0.2, 20.42±0.2, 21.58±0.2, 21.23±0.2, 23.20±0.2, 23.78±0.2, 24.58±0.2, 25.00±0.2, 25.78±0.2, 26.26±0.2, 26.63±0.2, 27.27±0.2, 28.10±0.2, 28.71±0.2, 30.00±0.2, 30.41±0.2, 31.08±0.2, 31.77±0.2, 32.42±0.2, 32.95±0.2, 33.50±0.2, 34.09±0.2, 34.74±0.2, 35.27±0.2, 36.21±0.2, 37.30±0.2, 38.11±0.2, 38.77±0.2.

In some embodiments, polymorphic Form B of freebase Compound (I) has an X-ray powder diffraction pattern comprising thirty or more peaks, in 2-theta values, wherein the thirty or more peaks are selected from 9.81±0.2, 8.95±0.2, 10.20±0.2, 13.26±0.2, 13.99±0.2, 17.21±0.2, 17.66±0.2, 18.55±0.2, 19.15±0.2, 20.42±0.2, 21.58±0.2, 21.23±0.2, 23.20±0.2, 23.78±0.2, 24.58±0.2, 25.00±0.2, 25.78±0.2, 26.26±0.2, 26.63±0.2, 27.27±0.2, 28.10±0.2, 28.71±0.2, 30.00±0.2, 30.41±0.2, 31.08±0.2, 31.77±0.2, 32.42±0.2, 32.95±0.2, 33.50±0.2, 34.09±0.2, 34.74±0.2, 35.27±0.2, 36.21±0.2, 37.30±0.2, 38.11±0.2, 38.77±0.2.

In some embodiments, polymorphic Form B of freebase Compound (I) has an X-ray powder diffraction pattern comprising thirty-four or more peaks, in 2-theta values, wherein the thirty-four or more peaks are selected from 9.81±0.2, 8.95±0.2, 10.20±0.2, 13.26±0.2, 13.99±0.2, 17.21±0.2, 17.66±0.2, 18.55±0.2, 19.15±0.2, 20.42±0.2, 21.58±0.2, 21.23±0.2, 23.20±0.2, 23.78±0.2, 24.58±0.2, 25.00±0.2, 25.78±0.2, 26.26±0.2, 26.63±0.2, 27.27±0.2, 28.10±0.2, 28.71±0.2, 30.00±0.2, 30.41±0.2, 31.08±0.2, 31.77±0.2, 32.42±0.2, 32.95±0.2, 33.50±0.2, 34.09±0.2, 34.74±0.2, 35.27±0.2, 36.21±0.2, 37.30±0.2, 38.11±0.2, 38.77±0.2.

In some embodiments, polymorphic Form B of freebase Compound (I) is characterized by an X-ray powder diffraction pattern having characteristic peaks expressed, in degree 2θ, at approximately 9.81±0.2, 8.95±0.2, 10.20±0.2, 13.26±0.2, 13.99±0.2, 17.21±0.2, 17.66±0.2, 18.55±0.2, 19.15±0.2, 20.42±0.2, 21.58±0.2, 21.23±0.2, 23.20±0.2, 23.78±0.2, 24.58±0.2, 25.00±0.2, 25.78±0.2, 26.26±0.2, 26.63±0.2, 27.27±0.2, 28.10±0.2, 28.71±0.2, 30.00±0.2, 30.41±0.2, 31.08±0.2, 31.77±0.2, 32.42±0.2, 32.95±0.2, 33.50±0.2, 34.09±0.2, 34.74±0.2, 35.27±0.2, 36.21±0.2, 37.30±0.2, 38.11±0.2, 38.77±0.2.

In some embodiments, polymorphic Form B of freebase Compound (I) is characterized by at least eight peaks in an X-ray powder diffraction pattern expressed, in degree 2θ, at approximately 8.8±0.2, 9.7±0.2, 10.1±0.2, 13.1±0.2, 13.9±0.2, 15.6±0.2, 18.4±0.2, and 21.4±0.2.

In some embodiments, polymorphic Form B of freebase Compound (I) characterized by an X-ray powder diffraction pattern having characteristic peaks expressed, in degree 2θ, as shown in Table 4 below.

TABLE 4

| Index | Position | Net Intensity |
|---|---|---|
| 1 | 8.952 | 809.565 |
| 2 | 9.808 | 1791.44 |
| 3 | 10.199 | 463.703 |
| 4 | 13.260 | 5171.93 |
| 5 | 13.985 | 1415.06 |
| 6 | 15.684 | 2598.42 |
| 7 | 17.210 | 318.841 |
| 8 | 17.662 | 651.663 |
| 9 | 18.546 | 328.574 |
| 10 | 19.146 | 659.461 |
| 11 | 20.423 | 115.703 |
| 12 | 21.226 | 819.012 |
| 13 | 21.577 | 3205.55 |
| 14 | 23.200 | 853.925 |
| 15 | 23.780 | 4531.92 |
| 16 | 24.579 | 78.0296 |
| 17 | 24.996 | 67.2932 |
| 18 | 25.778 | 1069.79 |
| 19 | 26.260 | 3983.35 |
| 20 | 26.631 | 1540.77 |
| 21 | 27.271 | 2657.26 |
| 22 | 28.100 | 1775.41 |
| 23 | 28.709 | 542.840 |
| 24 | 29.996 | 777.058 |
| 25 | 30.407 | 324.405 |
| 26 | 31.082 | 1051.01 |
| 27 | 31.765 | 834.376 |
| 28 | 32.419 | 126.795 |
| 29 | 32.950 | 816.260 |
| 30 | 33.497 | 159.299 |
| 31 | 34.085 | 184.121 |
| 32 | 34.738 | 715.696 |
| 33 | 35.265 | 457.310 |
| 34 | 36.208 | 288.512 |
| 35 | 37.297 | 44.8912 |
| 36 | 38.110 | 262.917 |
| 37 | 38.770 | 61.3028. |

In some embodiments, polymorphic Form B of freebase Compound (I) characterized by an X-ray powder diffraction pattern having characteristic peaks expressed, in degree 2θ, as shown in Table 5 below.

TABLE 5

| Index | Position | Rel. Intensity |
|---|---|---|
| 1 | 8.952 | 15.7% |
| 2 | 9.808 | 34.6% |
| 3 | 10.199 | 9.0% |
| 4 | 13.260 | 100.0% |
| 5 | 13.985 | 27.4% |
| 6 | 15.684 | 50.2% |
| 7 | 17.210 | 6.2% |
| 8 | 17.662 | 12.6% |
| 9 | 18.546 | 6.4% |
| 10 | 19.146 | 12.8% |
| 11 | 20.423 | 2.2% |
| 12 | 21.226 | 15.8% |
| 13 | 21.577 | 62.0% |
| 14 | 23.200 | 16.5% |
| 15 | 23.780 | 87.6% |
| 16 | 24.579 | 1.5% |
| 17 | 24.996 | 1.3% |
| 18 | 25.778 | 20.7% |
| 19 | 26.260 | 77.0% |
| 20 | 26.631 | 29.8% |
| 21 | 27.271 | 51.4% |
| 22 | 28.100 | 34.3% |
| 23 | 28.709 | 10.5% |
| 24 | 29.996 | 15.0% |
| 25 | 30.407 | 6.3% |
| 26 | 31.082 | 20.3% |
| 27 | 31.765 | 16.1% |
| 28 | 32.419 | 2.5% |
| 29 | 32.950 | 15.8% |
| 30 | 33.497 | 3.1% |
| 31 | 34.085 | 3.6% |
| 32 | 34.738 | 13.8% |
| 33 | 35.265 | 8.8% |
| 34 | 36.208 | 5.6% |
| 35 | 37.297 | 0.9% |
| 36 | 38.110 | 5.1% |
| 37 | 38.770 | 1.2%. |

In some embodiments, polymorphic Form B of freebase Compound (I) characterized by an X-ray powder diffraction pattern having characteristic peaks expressed, in degree 2θ, as shown in Table 6 below.

TABLE 6

| | | | Net | Gross | Rel. |
|---|---|---|---|---|---|
| Index | Position | d Value | Intensity | Intensity | Intensity |
| 1 | 8.952 | 9.87086 Å | 809.565 | 921.825 | 15.7% |
| 2 | 9.808 | 9.01122 Å | 1791.44 | 1909.95 | 34.6% |
| 3 | 10.199 | 8.66627 Å | 463.703 | 580.660 | 9.0% |
| 4 | 13.260 | 6.67191 Å | 5171.93 | 5328.07 | 100.0% |
| 5 | 13.985 | 6.32760 Å | 1415.06 | 1570.78 | 27.4% |
| 6 | 15.684 | 5.64559 Å | 2598.42 | 2760.86 | 50.2% |
| 7 | 17.210 | 5.14825 Å | 318.841 | 480.746 | 6.2% |
| 8 | 17.662 | 5.01766 Å | 651.663 | 817.057 | 12.6% |
| 9 | 18.546 | 4.78038 Å | 328.574 | 493.827 | 6.4% |
| 10 | 19.146 | 4.63189 Å | 659.461 | 822.162 | 12.8% |
| 11 | 20.423 | 4.34507 Å | 115.703 | 275.839 | 2.2% |
| 12 | 21.226 | 4.18254 Å | 819.012 | 1011.17 | 15.8% |
| 13 | 21.577 | 4.11513 Å | 3205.55 | 3408.14 | 62.0% |
| 14 | 23.200 | 3.83080 Å | 853.925 | 1109.21 | 16.5% |
| 15 | 23.780 | 3.73878 Å | 4531.92 | 4808.38 | 87.6% |
| 16 | 24.579 | 3.61895 Å | 78.0296 | 373.936 | 1.5% |
| 17 | 24.996 | 3.55945 Å | 67.2932 | 368.846 | 1.3% |
| 18 | 25.778 | 3.45324 Å | 1069.79 | 1387.71 | 20.7% |
| 19 | 26.260 | 3.39101 Å | 3983.35 | 4308.41 | 77.0% |
| 20 | 26.631 | 3.34464 Å | 1540.77 | 1868.54 | 29.8% |
| 21 | 27.271 | 3.26754 Å | 2657.26 | 2983.95 | 51.4% |
| 22 | 28.100 | 3.17302 Å | 1775.41 | 2089.90 | 34.3% |
| 23 | 28.709 | 3.10702 Å | 542.840 | 840.576 | 10.5% |
| 24 | 29.996 | 2.97664 Å | 777.058 | 1084.64 | 15.0% |
| 25 | 30.407 | 2.93729 Å | 324.405 | 645.991 | 6.3% |
| 26 | 31.082 | 2.87499 Å | 1051.01 | 1389.06 | 20.3% |
| 27 | 31.765 | 2.81471 Å | 834.376 | 1180.85 | 16.1% |
| 28 | 32.419 | 2.75947 Å | 126.795 | 473.576 | 2.5% |
| 29 | 32.950 | 2.71617 Å | 816.260 | 1157.70 | 15.8% |
| 30 | 33.497 | 2.67304 Å | 159.299 | 490.012 | 3.1% |
| 31 | 34.085 | 2.62826 Å | 184.121 | 508.439 | 3.6% |
| 32 | 34.738 | 2.58039 Å | 715.696 | 1038.84 | 13.8% |
| 33 | 35.265 | 2.54300 Å | 457.310 | 773.989 | 8.8% |
| 34 | 36.208 | 2.47888 Å | 288.512 | 584.126 | 5.6% |
| 35 | 37.297 | 2.40901 Å | 44.8912 | 323.722 | 0.9% |
| 36 | 38.110 | 2.35943 Å | 262.917 | 545.364 | 5.1% |
| 37 | 38.770 | 2.32075 Å | 61.3028 | 338.047 | 1.2%. |

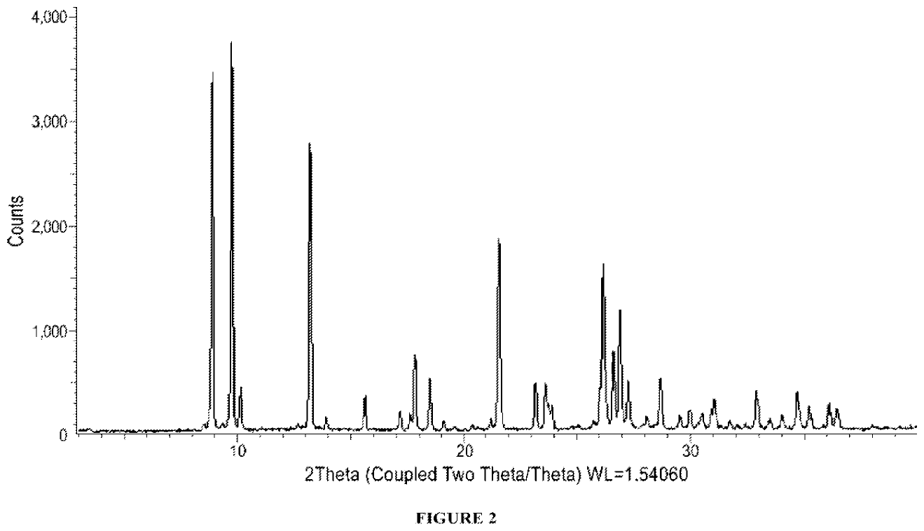

In some embodiments, polymorphic Form B of freebase Compound (I) has XRPD pattern as shown in FIG. 2.

In some embodiments, polymorphic Form B of freebase Compound (I) has a differential scanning calorimetry (DSC) thermogram profile characterized by an initial endothermic transition at about 182.3 °C±3 and a peak temperature at about 184 °C±3.

In some embodiments, polymorphic Form B of freebase Compound (I) has a thermogravimetric analysis (TGA) profile characterized by about 0.584% of weight loss at 175 °C±3.

In some embodiments, polymorphic Form B of freebase Compound (I) shows 1H NMR (DMSO-d6): δ 8.792 (d, J=2.5 Hz, 1H), 8.572 (dd, J=9.5, 2.5 Hz, 1H); 7.832 (D, J=9.5 Hz, 1H, 7.399 (s, 1H), 5.66.3 (s, 2H), 3.957 (s, 3H).

In some embodiments, polymorphic Form B of freebase Compound (I) has 13C NMR spectrum as shown in FIG. 9.

In some embodiments, polymorphic Form B of freebase Compound (I) is substantially free of impurities. In some embodiments, polymorphic Form B of freebase Compound (I) contains residual DNFB of less than 6 ppm, less than 5 ppm, less than 4 ppm, less than 3 ppm, less than 2 ppm, or less than 1 ppm. In some embodiments, polymorphic Form B of freebase Compound (I) contains residual DNFB of 1 ppm to 6 ppm, of 1 ppm to 5 ppm, of 1 ppm to 4 ppm, of 1 ppm to 3 ppm, or of 1 ppm to 2 ppm.

In some embodiments, polymorphic forms of freebase Compound (I) can be prepared by crystallization at room temperature by slow evaporation.

In some embodiments, polymorphic forms of freebase Compound (I) can be prepared by crystallization from hot saturated solutions by slow cooling.

In some embodiments, polymorphic forms of freebase Compound (I) can be prepared by crystallization from adding one or more anti-solvent.

In some embodiments, polymorphic Form B of freebase Compound (I) can be prepared by crystallization from heating a solution of polymorphic Form A.

In some embodiments, polymorphic Form B of freebase Compound (I) can be prepared by crystallization from thermal cycling a solution of polymorphic Form A.

In some embodiments, a small amount of polymorphic form of freebase Compound (I) to the above methods as seeding material.

In some embodiments, the suitable solvent is, including but not limiting to, alcohol solvents, acetone, acetonitrile, THF, ethyl acetate, isopropyl acetate, DCM, MEK, MTBE, n-heptane, 2-MeTHF, toluene, 1,4-dioxane, DMF, DMSO, or a mixture thereof.

In some embodiments, alcoholic solvents comprises methanol, ethanol, propanol, and the like.

In some embodiments, the solvent is DMSO.

In some embodiments, the solvent is DMF.

In some embodiments, the suitable anti-solvent is H2O, isopropyl acetate, MTBE, n-heptane, toluene, ethanol, or a mixture thereof.

In some embodiments, the solvent is DMF.

In some embodiments, the solvent is acetonitrile.

In some embodiments, the solvent is ethanol.

In some embodiments, the one or more acids are added to the solvent.

In some embodiments, the acid includes, but not limited to, HBr, HCl, or H2SO4.

In some embodiments, the mixture solution is heated to a temperature at above 35 °C, above 40 °C, above 45 °C, above 50 °C, above 55 °C, above 60 °C, above 65 °C, above 70 °C, above 75 °C, above 80 °C, above 100 °C, above 120 °C, above 140 °C, above 160 °C, or above 180 °C.

In some embodiments, the mixture solution comprising a polymorphic form is heated to a temperature between 35 °C and 60 °C, or between 35 °C and 50 °C, or between 40 °C and 60 °C, or between 60 °C and 80 °C, or between 65 °C and 70 °C.

In some embodiments, polymorphic Form A of freebase Compound (I) can be prepared by the following methods:
i. mixing freebase Compound (I) with a solvent, to form a mixture,
ii. optionally, adding one or more anti-solvents to the mixture,
iii. collecting crystalline Form A by filtration or slow evaporation, or heating the mixture to an elevated temperature then cooling the mixture to room temperature or 5 °C.

In some embodiments of the above methods, the solvent can be selected from acetone, acetonitrile, THF, DCM, MEK, 1,4-dioxane, DMF, DMSO, or a mixture thereof, the anti-solvent can be selected from H2O, isopropyl acetate, MTBE, n-heptane, toluene, or ethanol, and the elevated temperature is above 50 °C, above 60 °C, above 70 °C, above 80 °C, above 100 °C, above 120 °C, above 140 °C, or above 160 °C.

In some embodiments, polymorphic forms of freebase Compound (I) can be prepared by the following methods:
i. mixing freebase Compound (I) with a solvent to form a mixture,
ii. filtering the mixture,
iii. collecting crystalline Form A by slow evaporation from the mixture.

In some embodiments of the above methods, the solvent can be selected from acetone, acetonitrile, THF, DCM, MEK, 1,4-dioxane, DMF, DMSO, or a mixture thereof.

In some embodiments, polymorphic forms of freebase Compound (I) can be prepared by the following methods:
i. mixing freebase Compound (I) with 1,4-dioxane to form a mixture,
ii. filtering the mixture,
iii. collecting crystalline Form B by slow evaporation from the mixture.

In some embodiments, polymorphic Form A of freebase Compound (I) can be prepared by the following methods:
i. mixing freebase Compound (I) with a solvent to form a mixture,
ii. heating the mixture to an elevated temperature then cooling the mixture to 5 °C.

In some embodiments, the solvent can be selected from acetone, acetonitrile, THF, DCM, MEK, 1,4-dioxane, DMF, DMSO, water, or a mixture thereof. In some embodiments, the elevated temperature is above 50 °C.

In some embodiments, polymorphic forms of freebase Compound (I) can be prepared by the following methods:
i. mixing freebase Compound (I) with a solvent to form a mixture,
ii. optionally, adding one or more anti-solvents to the mixture,
iii. collecting crystalline Form B by filtration after heating the mixture to an elevated temperature,
iv. then cooling and promptly filtering the mixture.

In some embodiments of the above methods, the solvent can be selected from acetone, acetonitrile, THF, DCM, MEK, 1,4-dioxane, DMF, DMSO, or a mixture thereof, the anti-solvent can be selected from H2O, isopropyl acetate, MTBE, n-heptane, toluene, or ethanol, the elevated temperature is at 50 °C, the stirring can be at the elevated temperature or at room temperature or at 5 °C.

In some embodiments, polymorphic forms of freebase Compound (I) can be prepared by the following methods:
i. mixing freebase Compound (I) with a solvent to form a mixture,
ii. adding one or more anti-solvents to the mixture, and
iii. collecting crystalline Form A or B by filtration.

In some embodiments, the solvent can be selected from acetone, acetonitrile, THF, DCM, MEK, 1,4-dioxane, DMF, DMSO, or a mixture thereof, the anti-solvent can be selected from H2O, isopropyl acetate, MTBE, n-heptane, toluene, or ethanol, the elevated temperature is at 50 °C.

In some embodiments, polymorphic forms of freebase Compound (I) can be prepared by the following methods:
i. mixing freebase Compound (I) with a solvent to form a mixture,
ii. optionally, adding one or more anti-solvents to the mixture,
iii. collecting the crystalline Form A by filtration or slow evaporation,
iv. mixing Form A with a second solvent to form a second mixture, and heating the second mixture to an elevated temperature,
v. cooling and filtering to collecting crystalline Form A,
vi. optionally, repeating steps iv and v.

In some embodiments of the above methods, the solvent can be selected from acetone, acetonitrile, THF, DCM, MEK, 1,4-dioxane, DMF, DMSO, or a mixture thereof, the second solvent can be selected from H2O, isopropyl acetate, MTBE, n-heptane, toluene, or ethanol, the elevated temperature is above 50 °C, above 60 °C, above 70 °C, above 80 °C, above 100 °C, above 120 °C, above 140 °C, or above 160 °C, the stirring can be at the elevated temperature or at the room temperature. In some embodiments, the solvent in step iv can be a different solvent from step i.

In some embodiments, polymorphic forms of freebase Compound (I) can be prepared by the following methods:
i. mixing freebase Compound (I) with a heated solvent to form a mixture,
ii. cooling slowly the mixture to room temperature,
iii. adding a solvent to the mixture to precipitate Form B,
iv. collecting the crystalline Form B by filtration,
iv. mixing Form A with a solvent to form a second mixture and heating the second mixture to an elevated temperature, and
v. cooling and filtering to collect crystalline Form B.

In some embodiments of the above methods, the solvent can be selected from acetone, acetonitrile, THF, DCM, MEK, 1,4-dioxane, DMF, DMSO, or a mixture thereof, the anti-solvent can be selected from H2O, ethyl acetate, or ethanol, the elevated temperature is above 50 °C, above 60 °C, above 70 °C, above 80 °C, above 100 °C, above 120 °C, above 140 °C, or above 160 °C, the stirring can be at the elevated temperature or at the room temperature.

In some embodiments, the solvent used in step iv can be a different solvent from step i.

In some embodiments, the solvent mixture in step i is acetonitrile and DMSO. In some embodiments, the solvent in step iv is acetonitrile.

### Micronized Crystalline Compound (I)

Another aspect of the present disclosure provides a micronized crystalline form of 5-[(2,4-dinitrophenoxy)methyl]-1-methyl-2-nitro-1H-imidazole having the structure:

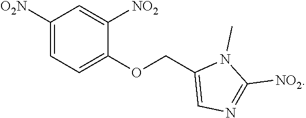 [SMILES: Cn1c(COc2ccc([N+](=O)[O-])cc2[N+](=O)[O-])cnc1[N+](=O)[O-]]

In some embodiments, the micronized crystalline form is micronized crystalline Form B.

In some embodiments, the micronized crystalline form is micronized crystalline Form A.

In some embodiments, crystalline Forms A and B are micronized. In some embodiments, the micronized crystalline form of Compound (I) is micronized crystalline Form A is micronized. In some embodiments, crystalline Form B is micronized.

In some embodiments, micronized crystalline Forms A and B have a particle size distribution (D10) of less than 4 μm. In some embodiments, micronized crystalline Forms A and B have a particle size distribution (D10) of between about 0.5 μm and about 4 μm, between about 0.5 μm to 3 μm, between about 0.5 μm to 2 μm, or between about 0.5 μm to 1.5 μm. In some embodiments, micronized crystalline Forms A and B have a particle size distribution (D10) of about 0.5 μm, 0.6 μm, 0.7 μm, 0.8 μm, 0.9 μm, 1.0 μm, 1.1 μm, or 1.2 μm.

In some embodiments, micronized crystalline Forms A and B have a particle size distribution (D50) of less than 50 μm. In some embodiments, micronized crystalline Forms A and B has a particle size distribution (D50) of less than 20 μm. In some embodiments, micronized crystalline Forms A and B has a particle size distribution (D50) of less than 15 μm. In some embodiments, micronized crystalline Forms A and B has a particle size distribution (D50) of less than 10 μm.

In some embodiments, micronized crystalline Forms A and B have a particle size distribution (D50) between about 0.5 μm and about 50 μm. In some embodiments, micronized crystalline Forms A and B have a particle size distribution (D50) between about 1 μm to 20 μm. In some embodiments, micronized crystalline Forms A and B have a particle size distribution (D50) between about 1 μm to 15 μm. In some embodiments, micronized crystalline Forms A and B have a particle size distribution (D50) between about 1 μm to 10 μm. In some embodiments, micronized crystalline Forms A and B have a particle size distribution (D50) between about 1 μm to 5 μm. In some embodiments, micronized crystalline Forms A and B have a particle size distribution (D50) between about 2 μm to 4 μm. In some embodiments, micronized crystalline Forms A and B have a particle size distribution (D50) between about 10 μm to 45 μm. In some embodiments, micronized crystalline Forms A and B have a particle size distribution (D50) between about 10 μm to 20 μm. In some embodiments, micronized crystalline Forms A and B have a particle size distribution (D50) about 12 μm.

In some embodiments, micronized crystalline Forms A and B have a particle size distribution (D50) of about 1, 1.1, 1.2, 1.3, 1.4, 1.5, 1.6, 1.7, 1.8, 1.9, 2, 2.5, 3, 3.5, or 4 μm. In some embodiments, micronized crystalline Forms A and B have a particle size distribution (D50) of about 3.5 μm. In some embodiments, micronized crystalline Forms A and B have a particle size distribution (D50) of about 3 μm. In some embodiments, micronized crystalline Forms A and B have a particle size distribution (D50) of about 1.8 μm. In some embodiments, micronized crystalline Forms A and B have a particle size distribution (D50) of about 1.2 μm. In some embodiments, micronized crystalline Forms A and B have a particle size distribution (D50) of about 1.24 μm, 1.75 μm, 12.06 μm, 19.63 μm, or 41.9 μm.

In some embodiments, micronized crystalline Forms A and B have a particle size distribution (D90) of less than 70 μm, less than 60 μm, less than 50 μm, less than 30 μm, or less than 20 μm. In some embodiments, micronized crystalline Forms A and B have a particle size distribution (D90) of between about 2 μm and about 60 μm, between about 2 μm to 55 μm, between about 1.0 μm to 10 μm, or between about 2.0 μm to 5.0 μm. In some embodiments, micronized crystalline Forms A and B have a particle size distribution (D90) of about 2.0 μm, 4.0 μm, 8.5 μm, or 53 μm.

In some embodiments, micronized crystalline Forms A and B have a particle size distribution (D90) of about 5 μm to about 15 μm. In some embodiments, micronized crystalline Forms A and B have a particle size distribution (D90) of about 8 μm to about 13 μm. In some embodiments, micronized crystalline Forms A and B have a particle size distribution (D90) of about 8, 9, 10, 11, 12, or 13 μm. In some embodiments, micronized crystalline Forms A and B have a particle size distribution (D90) of about 9 μm.

### Methods of Treatment

In some embodiments, the present disclosure provides a method for treating mitochondria-related disorders or conditions in a subject in need thereof comprising administering to the subject an effective amount of any of the crystalline forms of Compound (I) as described herein.

In some embodiments, the mitochondria-related disorders or condition is metabolic disorders, diabetes, or diabetes-associated complications.

In some embodiments, the disorder is obesity or excess body fat.

In some embodiments, the disorder is diabetes. In some embodiments, the disorder is type 2 diabetes (T2DM).

In some embodiments, the disorder is non-alcoholic fatty liver disease (NAFLD).

In some embodiments, the patient with NAFLD has elevated adiposity, or elevated HbA1c.

In some embodiments, the disorder is non-alcoholic steatohepatitis (NASH).

In some embodiments, the disorder is hepatic steatosis.

In some embodiments, the disorder is insulin resistance or intolerance.

In some embodiments, the disorder is dyslipidemia.

In some embodiments, the disorder is cardiovascular disease.

In some embodiments, the disorder is atherosclerosis.

In some embodiments, the present disclosure provides a method of reducing adiposity, controlling or preventing of weight gain in a subject in need thereof comprising administering to the subject an effective amount of any of the crystalline forms of Compound (I) as described herein.

In some embodiments, the present disclosure provides a method for stimulating oxygen consumption rate (OCR) in a subject in need thereof, comprising administering to the subject an effective amount of any of the crystalline forms of Compound (I) as described herein.

In some embodiments, the crystalline forms of 5-[(2,4-dinitrophenoxy)methyl]-1-methyl-2-nitro-1H-imidazole are useful for treating mitochondria-related disorders, including, but not limited to, obesity, diabetes, insulin resistance, and heart or renal failure in a subject in need thereof.

In some embodiments, the crystalline forms of 5-[(2,4-dinitrophenoxy)methyl]-1-methyl-2-nitro-1H-imidazole are useful for treating mitochondria-related disorders, including, is metabolic disorders, diabetes, or diabetes-associated complications.

In some embodiments, the crystalline forms of 5-[(2,4-dinitrophenoxy)methyl]-1-methyl-2-nitro-1H-imidazole are useful for controlling or preventing obesity or excess body fat in a subject in need thereof.

In some embodiments, the crystalline forms of 5-[(2,4-dinitrophenoxy)methyl]-1-methyl-2-nitro-1H-imidazole are useful for treating obesity or reducing adiposity in a subject in need thereof.

In some embodiments, the crystalline forms of 5-[(2,4-dinitrophenoxy)methyl]-1-methyl-2-nitro-1H-imidazole are useful for treating diabetes. In some embodiments, the crystalline forms of 5-[(2,4-dinitrophenoxy)methyl]-1-methyl-2-nitro-1H-imidazole are useful for treating type 2 diabetes (T2DM).

In some embodiments, the crystalline forms of 5-[(2,4-dinitrophenoxy)methyl]-1-methyl-2-nitro-1H-imidazole are useful for treating non-alcoholic fatty liver disease (NAFLD). In some embodiments, the crystalline forms of 5-[(2,4-dinitrophenoxy)methyl]-1-methyl-2-nitro-1H-imidazole are useful for treating non-alcoholic fatty liver disease (NAFLD), where the subject has elevated adiposity or elevated HbA1c.

In some embodiments, the crystalline forms of 5-[(2,4-dinitrophenoxy)methyl]-1-methyl-2-nitro-1H-imidazole are useful for treating non-alcoholic steatohepatitis (NASH).

In some embodiments, the crystalline forms of 5-[(2,4-dinitrophenoxy)methyl]-1-methyl-2-nitro-1H-imidazole are useful for treating hepatic steatosis.

In some embodiments, the crystalline forms of 5-[(2,4-dinitrophenoxy)methyl]-1-methyl-2-nitro-1H-imidazole are useful for treating insulin resistance or intolerance.

In some embodiments, the crystalline forms of 5-[(2,4-dinitrophenoxy)methyl]-1-methyl-2-nitro-1H-imidazole are useful for treating dyslipidemia.

In some embodiments, the crystalline forms of 5-[(2,4-dinitrophenoxy)methyl]-1-methyl-2-nitro-1H-imidazole are useful for treating cardiovascular disease.

In some embodiments, the crystalline forms of 5-[(2,4-dinitrophenoxy)methyl]-1-methyl-2-nitro-1H-imidazole are useful for treating atherosclerosis.

In some embodiments, the crystalline forms of 5-[(2,4-dinitrophenoxy)methyl]-1-methyl-2-nitro-1H-imidazole are useful for treating disease, disorders, and conditions which are associated with defects in mitochondrial function in a mammal in need thereof.

In some embodiments, the crystalline forms of 5-[(2,4-dinitrophenoxy)methyl]-1-methyl-2-nitro-1H-imidazole are useful for treating diabetes, including but not limiting, non-alcoholic fatty liver disease (NAFLD), non-alcoholic steatohepatitis (NASH), hepatic steatosis, and type 2 diabetes (T2DM) in a subject in need thereof.

In some embodiments, the crystalline forms of 5-[(2,4-dinitrophenoxy)methyl]-1-methyl-2-nitro-1H-imidazole are useful for controlling or preventing weight gain or maintaining of a weight in a subject in need thereof.

In some embodiments, the crystalline forms of 5-[(2,4-dinitrophenoxy)methyl]-1-methyl-2-nitro-1H-imidazole stimulate oxygen consumption rate (OCR) in a subject in need thereof.

In some embodiments, the crystalline forms of 5-[(2,4-dinitrophenoxy)methyl]-1-methyl-2-nitro-1H-imidazole are useful for treating cardiovascular disease in a subject in need thereof.

In some embodiments, the crystalline forms of 5-[(2,4-dinitrophenoxy)methyl]-1-methyl-2-nitro-1H-imidazole are useful for treating inflammation and fibrosis resulting in NASH.

In some embodiments, the present disclosure provides a method for treating above mentioned mitochondria-related disorders or conditions comprising administering micronized crystalline Compound (I) in a subject in need thereof.

In some embodiments, administering micronized crystalline Compound (I) extends the half-life (t_{1/2}) of Compound (I) compared to administering non-micronized crystalline Compound (I).

In some embodiments, administering micronized crystalline Compound (I) delays the time to maximum plasma concentration (Tₘₐₓ) of Compound (I). compared to administering non-micronized crystalline Compound (I).

In some embodiments, administering micronized crystalline Compound (I) lowers maximum plasma concentration (Cₘₐₓ) of Compound (I). compared to administering non-micronized crystalline Compound (I).

In some embodiments, administering micronized crystalline Compound (I) increases the area under the curve (AUC) of Compound (I) compared to administering non-micronized crystalline Compound (I).

In some embodiments, administering micronized crystalline Compound (I) extends the half-life (t_{1/2}) of 2,4-dinitrophenol compared to administering non-micronized crystalline Compound (I).

In some embodiments, administering micronized crystalline Compound (I) delays the time to maximum plasma concentration (Tₘₐₓ) of 2,4-dinitrophenol compared to administering non-micronized crystalline Compound (I).

In some embodiments, administering micronized crystalline Compound (I) lowers maximum plasma concentration (Cₘₐₓ) of 2,4-dinitrophenol compared to administering non-micronized crystalline Compound (I).

In some embodiments, administering micronized crystalline Compound (I) increases the area under the curve (AUC) of 2,4-dinitrophenol compared to administering non-micronized crystalline Compound (I).

### Pharmaceutical Compositions

One aspect of the disclosure provides a pharmaceutical composition comprising any of the crystalline forms described herein and a pharmaceutically acceptable carrier.

Pharmaceutical compositions for use in accordance with the present disclosure may be formulated in conventional manner using one or more pharmaceutically acceptable carriers comprising excipients and auxiliaries, which facilitate processing of the active compound into preparations that can be used pharmaceutically. Proper formulation is dependent upon the route of administration chosen. Pharmaceutically acceptable excipients and carriers are generally known to those skilled in the art. Such excipients and carriers are described, for example, Martin, Remington's Pharmaceutical Sciences, 15th Ed., Mack Publ. Co., Easton, PA (1975) or Rowe, Shesky, and Quinn, Handbook of Pharmaceutical Excipients, 6^{th} Ed. Pharmaceutical Press, London, UK (2009)).

In therapeutic use for controlling or preventing weight gain in a subject, the polymorphic forms of freebase Compound (I) is administered orally or parenterally.

In therapeutic use for treating mitochondria-related disorders or conditions in a subject, the polymorphic forms of freebase Compound (I) is administered orally or parenterally.

In therapeutic use for stimulating oxygen consumption rate (OCR) in a subject, the polymorphic forms of freebase Compound (I) is administered orally or parenterally.

In some embodiments, polymorphic forms of freebase Compound (I) or their pharmaceutical compositions are administered once, twice, or three times daily.

The amount of polymorphic freebase Compound (I) contained in the composition suitable for use in the present disclosure include an amount sufficient to achieve the intended purpose.

Determination of a therapeutically effective amount is well within the capability of those skilled in the art. Generally, the quantity of polymorphic freebase Compound (I) will range between 0.01% and 99.9% by weight of the composition. In some embodiments, the quantity of polymorphic freebase Compound (I) will range between 0.1% and 90% by weight of the composition. In some embodiments, the quantity of polymorphic freebase Compound (I) will range between 1% and 70% by weight of the composition. In some embodiments, the quantity of polymorphic freebase Compound (I) will range between 10% and 50% by weight of the composition.

A therapeutically effective amount of polymorphic freebase Compound (I) is in the range of about 0.001 to about 1000 mg/kg of body weight/day. The desired dosage may conveniently be presented in a single dose or divided doses administered at appropriate intervals, for example, as two, three, four or more sub-doses per day.

In some embodiments, the effective amount of polymorphic freebase Compound (I) is about 0.01 mg/kg to about 100 mg/kg.

In some embodiments, the effective amount of polymorphic freebase Compound (I) is between about 0.1 mg/kg to about 50 mg/kg and any and all whole or partial increments there between. For example, including but not limiting, about 0.1 mg/kg, about 1 mg/kg, about 10 mg/kg, about 100 mg/kg, about 200 mg/kg, or about 300 mg/kg.

In some embodiments, the effective amount of polymorphic freebase Compound (I) is about 1-10 mg/kg. In some embodiments, the effective amount of polymorphic freebase Compound (I) is about 2-10 mg/kg. In other embodiments, the effective amount of polymorphic freebase Compound (I) is about 3-10 mg/kg. In other embodiments, the effective amount of polymorphic freebase Compound (I) is about 4-10 mg/kg.

In some embodiments, the effective amount of polymorphic freebase Compound (I) is about 30 mg, 40 mg, 50 mg, 60 mg, 70 mg, 80 mg, 90 mg, 100 mg, 200 mg, 250 mg, 300 mg, 350 mg, 400 mg, 450 mg, 500 mg, 600 mg, 700 mg, 800 mg, 900 mg, 1000 mg, or 1050 mg.

In some embodiments, the effective amount of polymorphic freebase Compound (I) is about 2-10 mg/kg. In other embodiments, the effective amount of polymorphic freebase Compound (I) is about 3-10 mg/kg.

In other embodiments, the effective amount of polymorphic freebase Compound (I) is about 4-10 mg/kg.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

### EXAMPLES

### Definitions

Compound (I): 5-[(2,4-dinitrophenoxy)methyl]-1-methyl-2-nitro-1H-imidazole
MeOH: Methanol
EtOH: Ethanol
IPA: Isopropanol
MEK: Methyl ethyl ketone
ACN: Acetonitrile
THF: Tetrahydrofuran
EA: Ethyl acetate
MTBE: Methyl tert-butyl ether
DCM: Dichloromethane
2-MeTHF: 2-methyl tetrahydrofuran
DMSO: Dimethyl sulfoxide
XRPD: X-ray powder diffractometer
DSC: Differential scanning calorimetric
TGA: Thermal gravimetric analysis
DVS: Dynamic vapor sorption
NMR: Nuclear magnetic resonance
SEM: Scanning Electronic Microscope
FT-IR: Fourier Transform Infrared Spectrum
KF: Karl Fisher
HPLC: High Performance Liquid Chromatograph

### Example 1. Polymorph Screening

The objective of the polymorph study is to identify potential polymorphs and select an optimal polymorph in terms of stability, hygroscopicity and feasibility for downstream development. Freebase Compound (I) is poorly soluble. Crystalline forms increase solubility through different solubilizing agents.

Solubility of Compound (I) at pH 1-6.5 was equivalent, i.e. its solubility is not pH-dependent. Study results suggest that Compound (I) is not likely to be ionizable and successful salt formation was hypothesized to be unlikely.

However, precipitation of Compound (I) from solutions in EtOH with HBr and H₂SO₄ and with HCl in Acetone results in polymorphs not previously observed for freebase. Two distinct polymorphic forms, Form A and Form B were identified. Several isoforms were also identified.

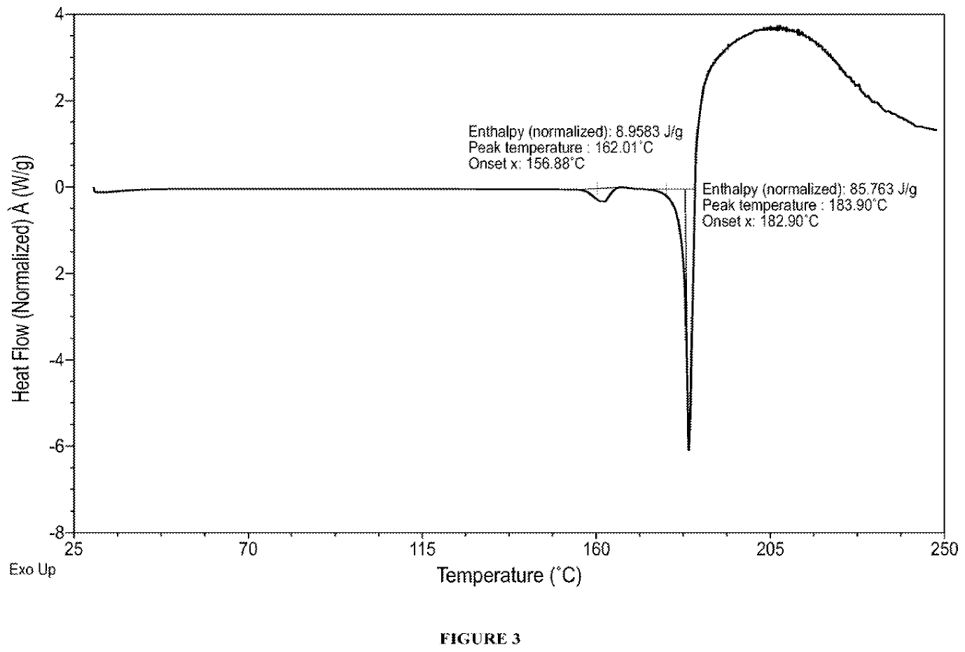

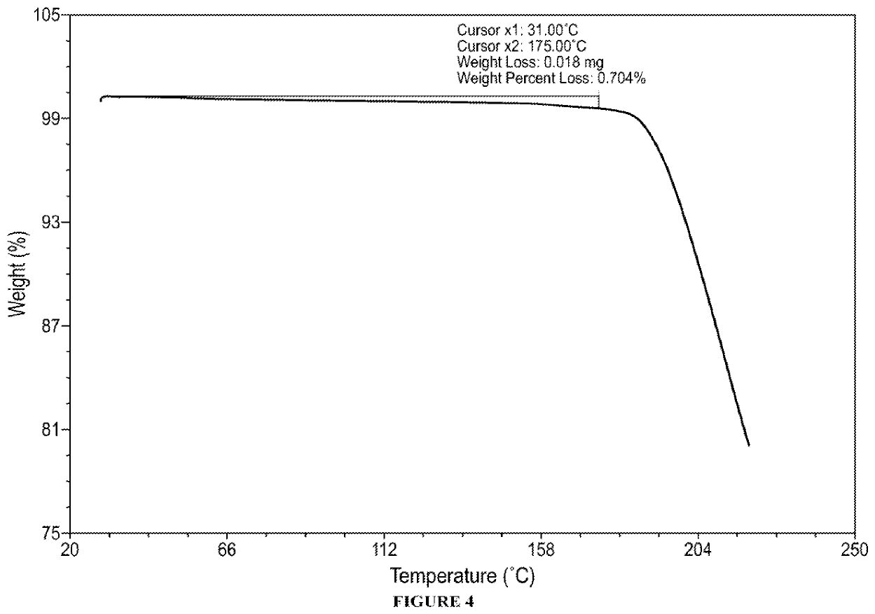

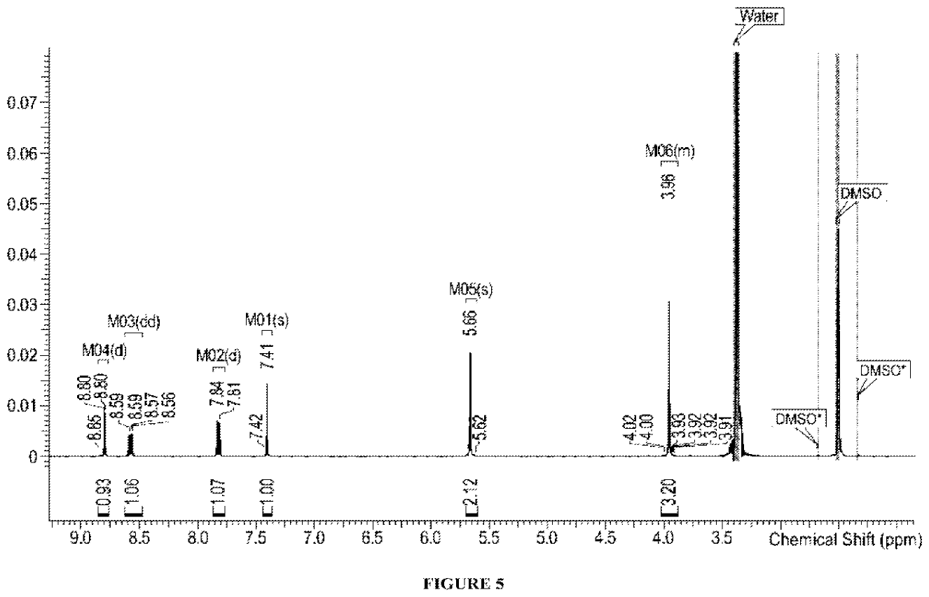

Form A is an anhydrate. It was obtained from most of solvent systems by equilibration, slow evaporation, slow cooling and anti-solvent addition. Form A, is of high crystallinity. DSC profile (FIG. 3) shows one endothermic peak at Tₒₙₛₑₜ of 156.9 °C with an enthalpy of 9 J/g. FIG. 3, corresponding to the solid-solid transition from Form A to Form B, and then a melting peak of Tₒₙₛₑₜ at 182.9 °C and enthalpy at 183.9 °C. Decomposition occurs upon the melting. TGA (FIG. 4) shows about 0.7% weight loss at about 175 °C. ¹H-NMR (FIG. 5) shows no detectable residual solvent. Form A is the thermodynamically stable anhydrate at 35 °C or below.

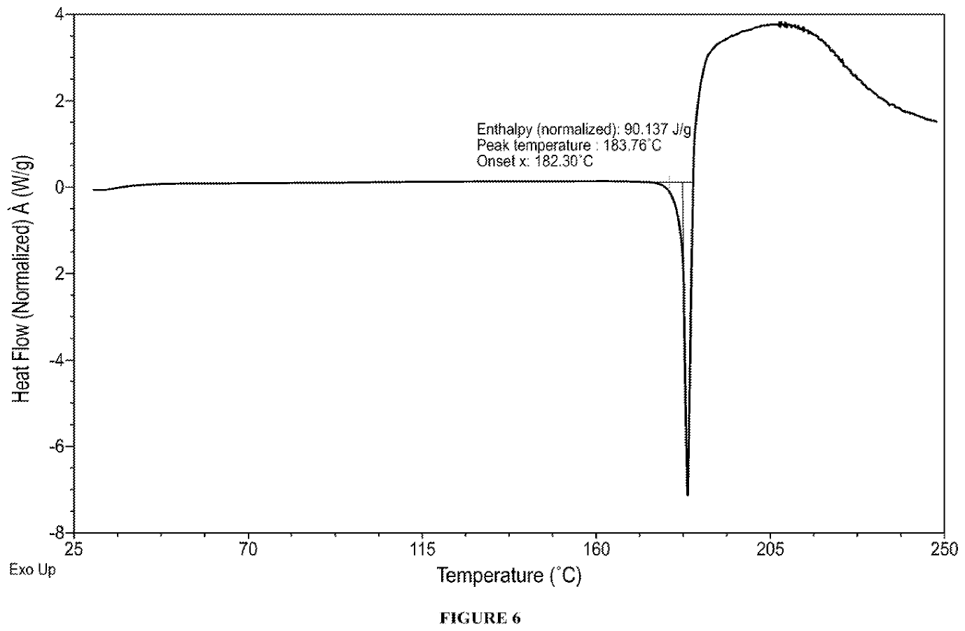

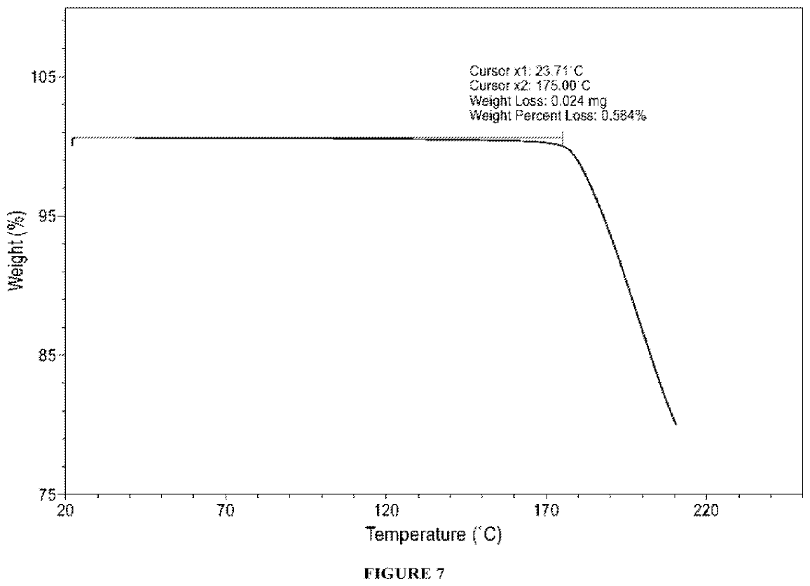

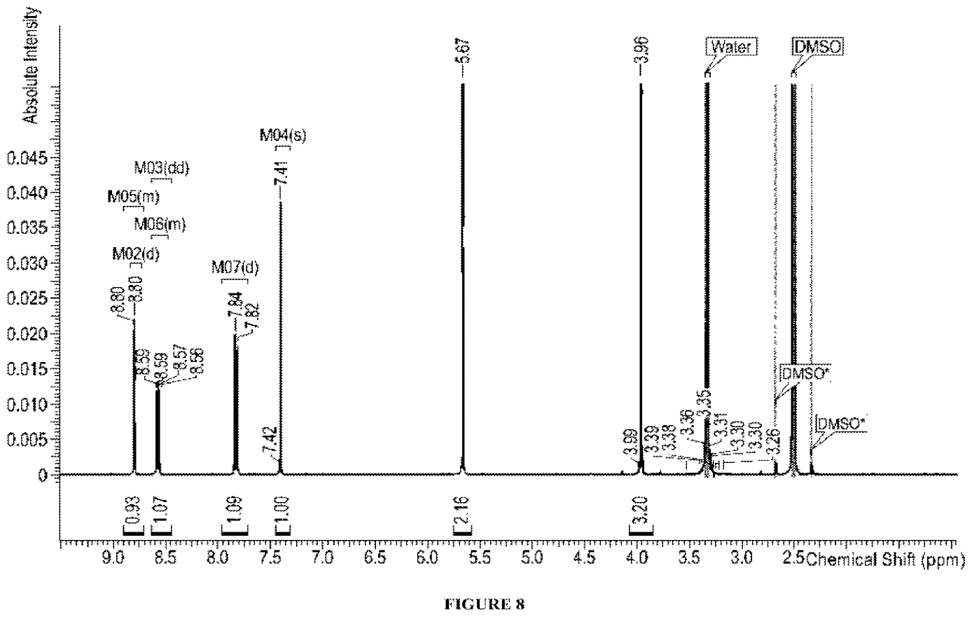

Form B is an anhydrate. It was obtained by heating Form A to 165 °C or by slow evaporation in 1,4-dioxane. Form B is of high crystallinity. DSC (FIG. 6) shows a melting peak at Tₒₙₛₑₜ of 182.3 °C. Decomposition occurs upon melting. TGA (FIG. 7) shows about 0.6% weight loss at about 175 °C. ¹H-NMR (FIG. 8) shows no detectable residual solvent. Form B is the thermodynamically stable Form A at 50 °C or above.

Based on competitive equilibration results, Form A+B are enantiotropically related. At 35 °C or below, Form A is the only polymorph or main polymorph in samples and therefore the thermodynamically stable Form A at 35 °C or below. At 50 °C, Form B is the only polymorph or main polymorph in samples and therefore the thermodynamically stable form at 50 °C or above. At 40 °C, Form A was obtained as the major product from acetone or THF, while Form B was obtained as the major product from acetonitrile/water (v:v=85:15) system, suggesting that the phase transition temperature is around 40 °C. Slight thermodynamic disturbance at this temperature may lead to different equilibrium results.

Based on competitive equilibration results, the thermodynamically stable anhydrate at ambient temperature (20-25 °C) is Form A.

Bulk stability of Form A and Form B of Compound (I) was evaluated at 25 °C/92% RH in an open container, at 40 °C/75% RH in an open container and at 60 °C in a tight container for 1 and 2 weeks. Form A and Form B of Compound (I) are physically and chemically stable under these conditions.

Hygroscopicity of both Form A and Form B of Compound (I) was evaluated by dynamic vapor sorption (DVS) test at 25 °C. Form A of Compound (I) is non-hygroscopic. It absorbs about 0.1% water from 40% RH to 95% RH at 25 °C. After the DVS test, obtained sample was still Form A. Form B of Compound (I) is non-hygroscopic. It absorbs about 0.1% water from 40% RH to 95% RH at 25 °C. After the DVS test, obtained sample was still Form B.

Form A is predominantly formed and stable at room temperatures. Form B is more stable at temperatures above (approximately) 37 °C. By heating the slurry, Form B is accumulated over time. By heating the slurry, more residual dinitrofluorobenzene (DNFB) can be removed, and the desired purity is achieved.

The removal of impurities is likely to be more effective in heated solvents, thus producing Form B at a higher temperature results in more residual impurities being removed.

Based on DSC analysis and competitive equilibration experiment results, Form A and Form B are enantiotropically related. Form A is the thermodynamically stable Form at 35 °C or below, while Form B is the thermodynamically stable Form at 50 °C or above. Both Form A and Form B show good chemical and physical stability and are non-hygroscopic at 25 °C. Although Form A and Form B show good chemical and physical stability after 2-week bulk stability study, this result only describes kinetic stability of the two interconvertible polymorphs as a bare drug substance for a short term. As revealed by the competitive equilibration experiments, given an accelerated kinetic condition and sufficient time, the two polymorphs will convert to each other depending on the temperature.

TABLE 7
Summary of characterization of polymorphs

| | Form A (anhydrate) | Form B (anhydrate) |
|---|---|---|
| Crystallinity | Highly crystalline | Highly crystalline |
| (by XRPD) | | |
| Melting onset | Endothermic onset: | Melting onset: |
| (by DSC, °C) | 156.9 °C; | 182.3 °C, |
| | Melting onset: 182.9 °C. | |
| Enthalpy | 9 J/g; | Decomposition upon |
| (by DSC, J/g) | Decomposition upon | melting |
| | melting. | |
| Weight loss (by TGA) | 0.7% at 175 °C | 0.6% at 175 °C |
| Residual solvent | No detectable residual | No detectable residual |
| (by ¹H-NMR) | solvent | solvent |
| | Form A converted to Form | Not Applicable |
| | B after heating to 165 °C | |

### Example 2. Solubility Study

About 5 mg of Form A was weighed to a 2 mL glass vial. 20 μL aliquots of each solvent were added to dissolve the drug substance at 25 °C. About 10 mg of Form A was weighed to a 2 mL glass vial. 20 μL aliquots of each solvent were to dissolve the drug substance at 50 °C. Sonication were applied to assist dissolution. Maximum volume of each solvent added is 1 mL. Approximate solubility was determined by visual observation.

TABLE 8
Solubility of Form A at 25 °C and at 50 °C

| | Solubility (mg/mL) | |
|---|---|---|
| Solvents | 25 °C | 50 °C |
| Water | <5 | <5 |
| Methanol | <5 | <5 |
| Ethanol | <5 | <5 |
| Acetone | 17-25 | 20-25 |
| Acetonitrile | 17-20 | 33-50 |
| THF | 13-14 | 22-25 |
| Ethyl acetate | <5 | 5-10 |
| Isopropyl acetate | <5 | <5 |
| DCM | ~5 | / |
| MEK | 8-9 | 20-25 |
| MTBE | <5 | / |
| Heptane | <5 | <5 |
| 2-MeTHF | <5 | <5 |
| Toluene | <5 | <5 |
| 1,4-Dioxane | 8-9 | 17-20 |
| DMSO | 125-250 | 250-500 |

Addition solubility of crystal forms were collected. Results showed that the solubility was low in all selected solvents except DMSO at 25 °C and 50 °C. Form A was used as the starting material for all the following studies. The results were listed in table 9.

TABLE 9
Solubility of Form A in single solvents

| Starting | | Solubility (mg/ml) | | Final |
|---|---|---|---|---|
| Material | Solvent | 25 °C | 50 °C | Material |
| Form A | MeOH | <5 | <5 | Form A |
| | EtOH | <5 | <5 | Form A |
| | EA | <5 | 5-10 | Form A |
| | IPAc | <5 | <5 | / |
| | Acetone | 17-25 | 20-25 | Form A |
| | MEK | 8-9 | 20-25 | Form A |
| | MTBE | <5 | // | / |
| | ACN | 17-20 | 33-50 | Form A |
| | DCM | ~5 | // | Form A |
| | THF | 13-14 | 22-25 | Form A |
| | 2-MeTHF | <5 | <5 | Form A |
| | Heptane | <5 | <5 | / |
| | H₂O | <5 | <5 | Form A |
| | DMSO | 125-250 | 250-500 | / |
| | Toluene | <5 | <5 | / |

The starting and final material were verified by XRPD. All XRPD profile were the same at 25 °C and 50 °C. Final material refers to the compounds after recrystallizing.

Some slurry experiments were carried out in different solvent systems with crystal forms. Results showed that the Forms A+B converted to Form B at 50-60 °C in acetone and MEK system. Form B was stable after slurrying in different solvents at different temperatures. The solubility by HPLC are listed in Tables 10 and 11.

TABLE 10
Solubility of crystal forms of
Compound (I) in the mixture solvents

| | | | Solubility | |
|---|---|---|---|---|
| Material | Solvent | T/°C | (mg/ml) | XRPD |
| Forms | DMSO/EA | 35 | 55 | Forms |
| A + B | (1/4 v/v) | 25 | 40 | A + B |
| | | 0-5 | 20-33 | |
| | DMSO/EA | 35 | 36 | |
| | (1/6 v/v) | 25 | 25 | |
| | | 0-5 | 17-25 | |
| | Acetone | 60 | 52 | Form B |
| | | 50 | / | Form B |
| | | 0-5 | 16 | Form A |
| | MEK | 60 | 39 | Form B |
| | | 0-5 | 8 | Form A |
| Form B | Acetone | 0-5 | 17 | Form B |
| | MEK | | 9 | Form B |

TABLE 11
Solubility of crystal forms of
Compound (I) in DMSO/H2O and

| Starting | | Ratio | | Solubility | Final |
|---|---|---|---|---|---|
| Material | Solvent | (v/v) | T/°C | (mg/ml) | Material |
| Form B | DMSO/H₂O | 9/1 | 50 | 80 | Form B |
| | | 2/1 | 50 | 1.6 | |
| | | | 25-30 | 0.5 | |
| | | 1/4 | 0-5 | 0 | |
| | DMSO/EtOH | 2/1 | 50 | 107 | |
| | | 1/1 | 50 | 39 | |
| | | 1/3 | 50 | 7 | |
| | | | 25-30 | 4 | |
| | | 1/4 | 0-5 | 1.4 | |

### Example 3. Water Sorption and Desorption Experiments of Polymorphic Form B

Water sorption and desorption behavior of Forms A and B were investigated by DVS at 25 °C with a cycle of 40-95-0-95-40% relative humidity (RH), equilibration time 240 min for each step. XRPD was measured after the DVS test to determine form change.

TABLE 12
Water sorption and desorption experiments of Form A
Method
40-95-0-95-40% RH,
equilibration time 240 min for each step, at 25 °C

| | 1^{st} sorp, | 1^{st} desorp, | 2^{nd} sorp, | 2^{nd} desorp, |
|---|---|---|---|---|
| Relative | weight | weight | weight | weight |
| humidity | % change | % change | % change | % change |
| 0% | N/A | 0.06 | 0.06 | Not applicable |
| 10% | N/A | 0.06 | 0.07 | Not applicable |
| 20% | N/A | 0.08 | 0.07 | Not applicable |
| 30% | N/A | 0.08 | 0.08 | Not applicable |
| 40% | 0.08 | 0.06 | 0.09 | 0.08 |
| 50% | 0.08 | 0.08 | 0.10 | 0.09 |
| 60% | 0.08 | 0.09 | 0.09 | 0.10 |
| 70% | 0.09 | 0.10 | 0.10 | 0.10 |
| 80% | 0.10 | 0.11 | 0.11 | 0.11 |
| 90% | 0.11 | 0.12 | 0.11 | 0.12 |
| 95% | 0.12 | 0.12 | 0.12 | 0.12 |

XRPD after DVS test
0.1% water uptake at 95% RH

TABLE 13
Water sorption and desorption experiments of Form B

| Method | 40-95-0-95-40%RH, equilibration time 240 min for each step, at 25 °C | | | |
|---|---|---|---|---|
| Relative | 1^{st} sorp, weight | 1^{st} desorp, weight | 2^{nd} sorp, weight | 2^{nd} desorp, weight |
| humidity | % change | % change | % change | % change |
| 0% | N/A | 0.09 | 0.09 | N/A Not applicable |
| 10% | N/A | 0.10 | 0.07 | Not applicable |
| 20% | N/A | 0.13 | 0.09 | Not applicable |
| 30% | N/A | 0.09 | 0.11 | Not applicable |
| 40% | 0.11 | 0.12 | 0.12 | 0.13 |
| 50% | 0.12 | 0.12 | 0.14 | 0.13 |
| 60% | 0.11 | 0.14 | 0.13 | 0.14 |
| 70% | 0.11 | 0.13 | 0.14 | 0.14 |
| 80% | 0.12 | 0.15 | 0.14 | 0.15 |
| 90% | 0.14 | 0.16 | 0.13 | 0.16 |
| 95% | 0.14 | 0.14 | 0.15 | 0.15 |

0.1% water uptake at 95% RH

### Example 4. Crystallization at Room Temperature by Slow Evaporation

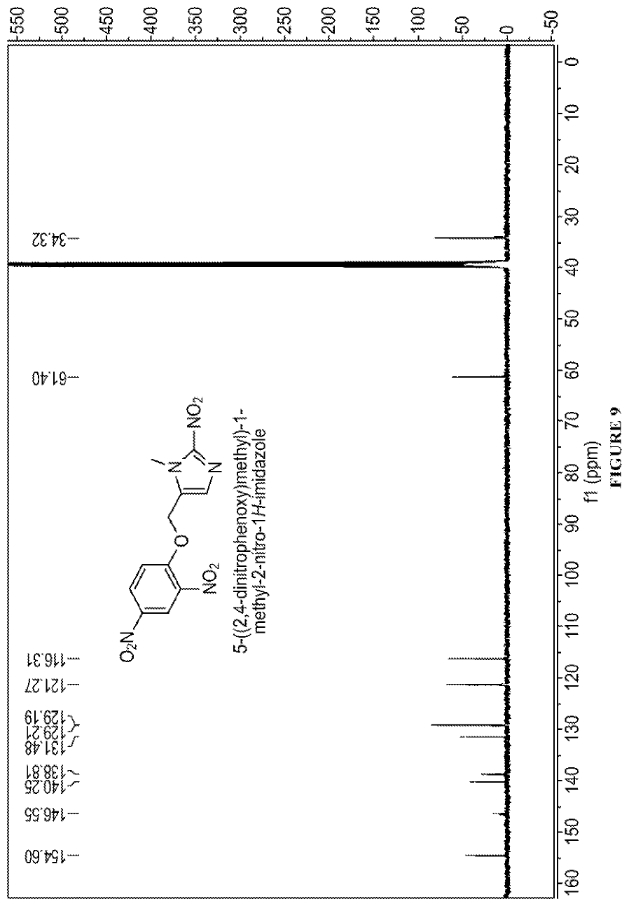

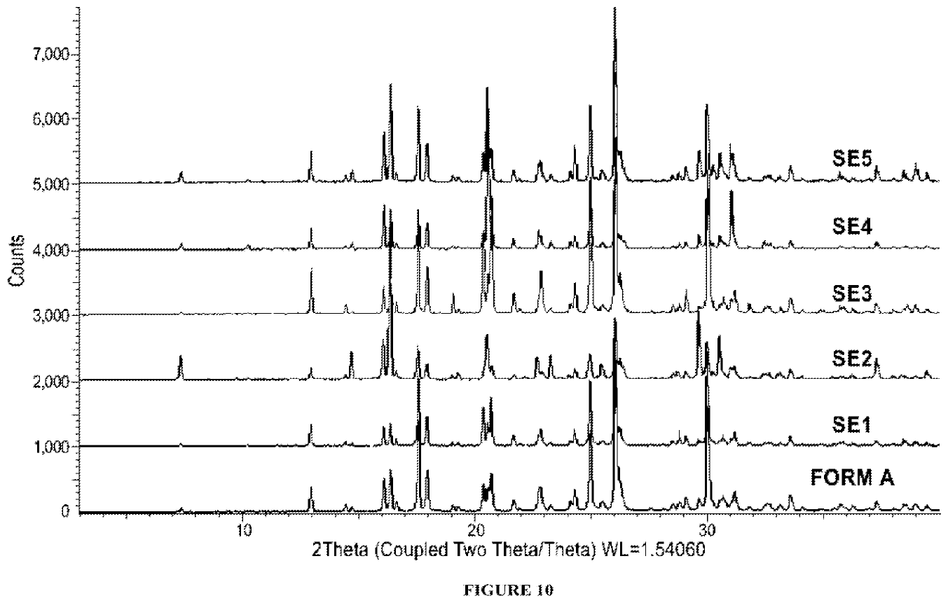

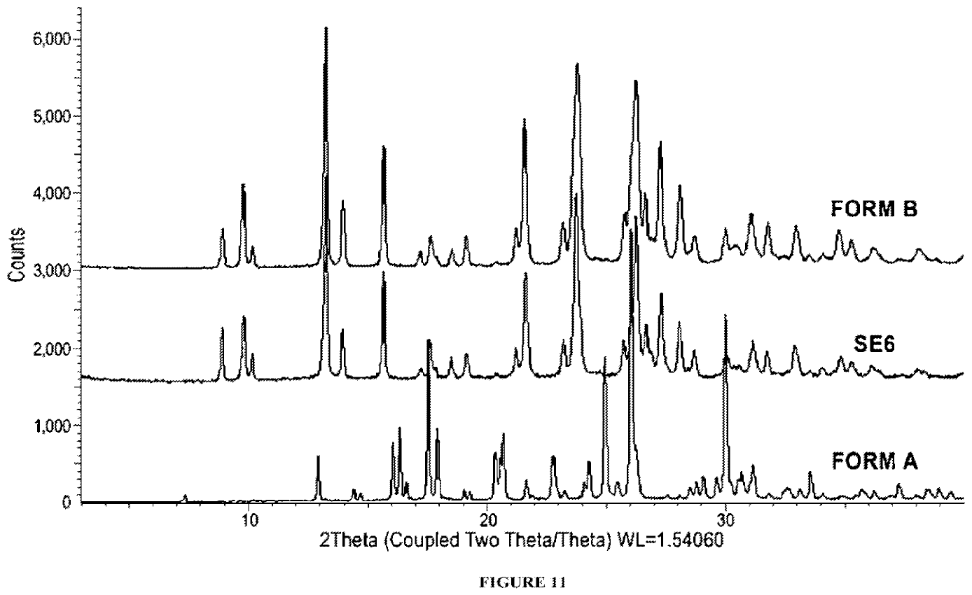

20 mg of Compound (I) free form was dissolved in 0.2-1 mL of solvents. Obtained solutions were filtered through a 0.45 μm nylon membrane filter by centrifugation at 14,000 rpm. Obtained clear solutions were slowly evaporated in ambient condition (about 20-25 °C; 40-60% RH). Solid residues were investigated by XRPD as shown in Table 14. FIG. 10 shows XRPD patterns of Form A obtained by this method. FIG. 11 shows XRPD patterns of Forms A and Form B obtained by this method.

TABLE 14
Crystallization at room temperature by slow evaporation

| Exp. ID | Solvents | XRPD |
|---|---|---|
| SE1 | Acetone | Form A |
| SE2 | Acetonitrile | Form A |
| SE3 | THF | Form A |
| SE4 | DCM | Form A |
| SE5 | MEK | Form A |
| SE6 | 1,4-Dioxane | Form B |

### Example 5. Crystallization from Hot Saturated Solutions by Slow Cooling

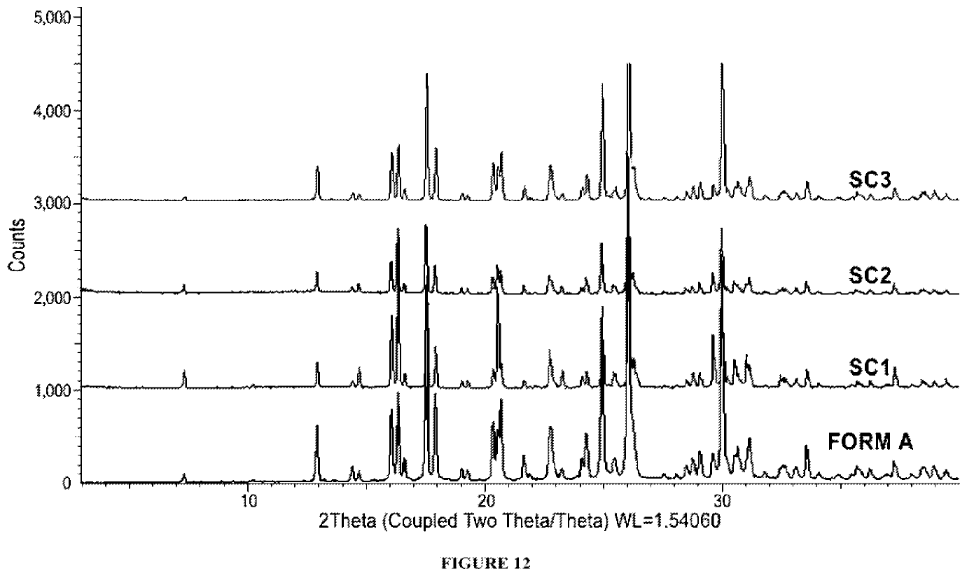

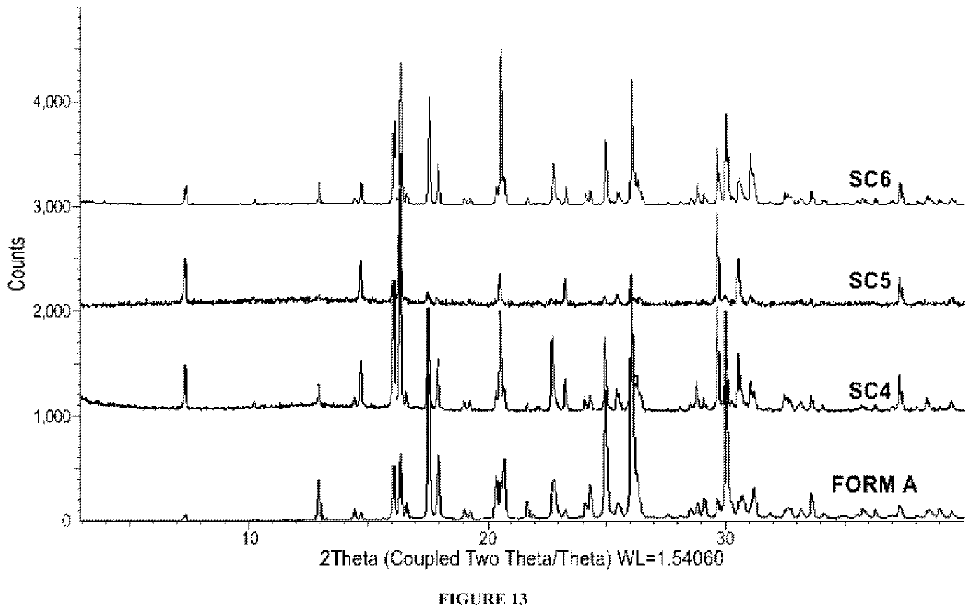

40 mg of Compound (I) free form was dissolved in the minimal amount of selected solvents at 50 °C. Obtained solutions were filtered through a 0.45 μm nylon membrane filter by centrifugation at 14,000 rpm. Obtained clear solutions were cooled to 5 °C at 0.1 °C/min. Precipitates were collected by centrifugation filtration through a 0.45 μm nylon membrane filter at 14,000 rpm. Solid parts (wet cakes) were investigated by XRPD. FIG. 12 shows XRPD patterns of Form A obtained by this method (SC1-SC3). FIG. 13 shows XRPD patterns of Form A obtained by this method (SC4-SC6).

TABLE 15
Crystallization from hot saturated solutions by slow cooling

| Exp. ID | Solvents | XRPD |
|---|---|---|
| SC1 | Acetonitrile | Form A |
| SC2 | Ethyl acetate | Form A |
| SC3 | MEK | Form A |
| SC4 | 1,4-Dioxane | Form A |
| SC5 | Acetone/water | Form A |
| | (v:v = 3:7) | |
| SC6 | Acetonitrile/water | Form A |
| | (v:v = 85:15) | |

### Example 6. Crystallization by Addition of Anti-Solvent

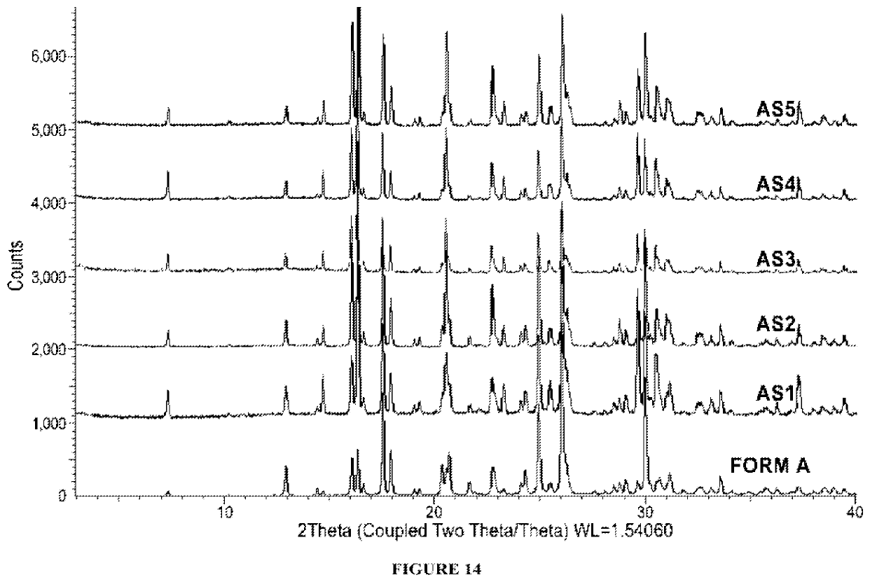

30 mg of Compound (I) free form was dissolved in the minimal amount of selected good solvents at ambient temperature (about 20-25 °C). 1-4 folds of anti-solvent were added into the obtained clear solutions slowly until a large amount of solids precipitated out. Precipitates were collected by centrifugation filtration through a 0.45 μm nylon membrane filter at 14,000 rpm. Solid parts (wet cakes) were investigated by XRPD. FIG. 14 shows XRPD patterns of Form A obtained by this method.

TABLE 16
Crystallization by addition of anti-solvent

| Exp. | | Anti- | |
|---|---|---|---|
| ID | Solvents (mL) | solvent (mL) | XRPD |
| AS1 | DMSO (0.24) | Water (0.24) | Form A |
| AS2 | Acetonitrile (1.5) | Water (1.5) | Form A |
| AS3 | THF (2.0) | Isopropyl | Form A |
| | | acetate (6) | |
| AS4 | Acetone (1.6) | MTBE (6.4) | Form A |
| AS5 | Acetone (1.6) | Heptane (2.4) | Form A |
| AS6 | Acetone (1.6) | Toluene (6.5) | // |

“//” XRPD not conducted;
Few solids produced, not enough for XRPD analysis.

### Example 7. Production of Form B of Compound (I)

Compound (I) in DMF solution is charged to a reactor and polish filtered and stored in clean containers, resulting in clarified Compound (I). Half of the clarified Compound (I) in DMF to be processed is charged to a reactor. Water is then added to the clarified filtrate to precipitate the crude Compound (I) product, which is stirred, and isolated by filtration. This procedure is repeated for the second half of the clarified Compound (I). The combined crude wetcakes are washed with ethanol. The crude wetcake is partially dried in the filter, added back to the reactor, slurried again in water at 35-45 °C, cooled, and stirred at room temperature. The crude Compound (I) is filtered, washed with ethanol and partially dried in the filter. The crude Compound (I) is suspended in ethanol and heated to 60-70 °C for 2-3 hours before being cooled to room temperature, filtered, washed with ethanol, and partially dried. The resulting solid is dried under vacuum overnight then recrystallized by heating in acetonitrile to 60-70 °C for 16-20 hours and slowly cooling to room temperature. The resulting crystals, Form B, are filtered, washed with ethanol and dried until a constant weight is achieved. The dried crystals, Form B, are then sieved with a 40 mesh sieve before packaging.

¹H NMR (DMSO-d6): δ 8.792 (d, J=2.5 Hz, 1H), 8.572 (dd, J=9.5, 2.5 Hz, 1H); 7.832 (D, J=9.5 Hz, 1H, 7.399 (s, 1H), 5.66.3 (s, 2H), 3.957 (s, 3H).

### Example 7.1. Production of Form B of Compound (I)

Form A of Compound (I) was dissolved in a heated mixture of ACN:DMSO in a glass lined reactor, polish filtered (rinsed forward with ACN) and cooled slowly to 25 °C over no less than 4 hours. After cooling to room temperature, water was added over no less than 4 h to complete the product precipitation. After agitating for not less than 1 hour, solids were isolated by filtration, rinsed with EtOH, and dried in a vacuum oven at 45 °C to constant mass. The dried solids were then analyzed for various critical quality attributes (second row of Table 17).

Acetonitrile slurry: The dried solids were slurred in acetonitrile at 70 °C. After agitating for no less than 16 hours, the mixture was then cooled to 25 °C and the product was isolated by centrifugation. The solids were rinsed with EtOH and dried to constant mass to isolate Form B (65%).

As shown in Table 17, the acetonitrile reslurry also purged impurities from Form B, particularly residual DNFB from 12.3 to 1.9 ppm (the limit is 6 ppm).

TABLE 17
Impurities quantification

| | Residual | | | | | | | |
|---|---|---|---|---|---|---|---|---|
| Notes | DNFB^{a} | XRPD | ACN^{a} | DMSO^{a} | TEA^{a} | EtOH^{a} | THF^{a} | Toluene^{a} |
| Form A of | 1113.0 | Form A | ND | 520 | 212 | 7 | 4 | ND |
| Compound (I) | | | | | | | | |
| First dried solids | 12.3 | Forms | 780 | 77 | ND | 12 | 3 | ND |
| | | A and B | | | | | | |
| Acetonitrile Slurry | 1.9 | Form B | 381 | ND | ND | 31 | ND | ND |

^{a}all values listed are in units of ppm.

### Example 8. Single Crystal Cultivation and Analysis

The purpose of this study is to use single crystal analysis to resolve single crystal structures of Form A and Form B.

Instrumental methods are listed below:

X-ray Powder Diffractometer (XRPD)

| | |
|---|---|
| Instrument | Bruker D8 Advance |

XRPD method

| | |
|---|---|
| X-ray geometry | Reflection |
| Detector | LYNXEYE XE T(ID mode) |
| Open angle | 2.94° |
| Radiation | Cu/K-Alpha1 (2=1.5406Å) |
| X-ray generator power | 40 kV, 40 mA |
| Primary beam path slits | Twin Primary motorized slit 10.0 mm by |
| | sample length; SollerMount axial soller 2.5° |
| Secondary beam path slits | Detector OpticsMount soller slit 2.5°; |
| | Twin_Secondary motorized slit 5.2 mm |
| Scan mode | Continuous scan |
| Scan type | Locked coupled |
| Step size | 0.2° |
| Time per step | 0.12 second per step |
| Scan range | 3° to 40° |
| Sample rotation speed | 15 rpm |
| Sample holder | Monocrystalline silicon, flat surface |

Single Crystal X-ray Diffractometer (SCXRD)

| | |
|---|---|
| Instrument | Bruker D8 Venture |

method

| | |
|---|---|
| Detector | CMOS area detector |
| Temperature | 170(2)K |
| Radiation | Cu/K-Alpha1 (λ = 1.5418 Å) |
| X-ray generator power | 50 kV, 10 mA |
| Distance from sample to | 40 mm |
| area detector | |
| Exposure time | 2 second |
| Resolution | 0.81 Å |

Polarized Light Microscope (PLM)

| | |
|---|---|
| Instrument | Nikon LV100POL |
| Method | Crossed polarizer, silicone oil added |

Single crystal Form A suitable for single crystal analysis were obtained from slow evaporation method in DCM. FIG. 1 shows XRPD pattern of Form A.

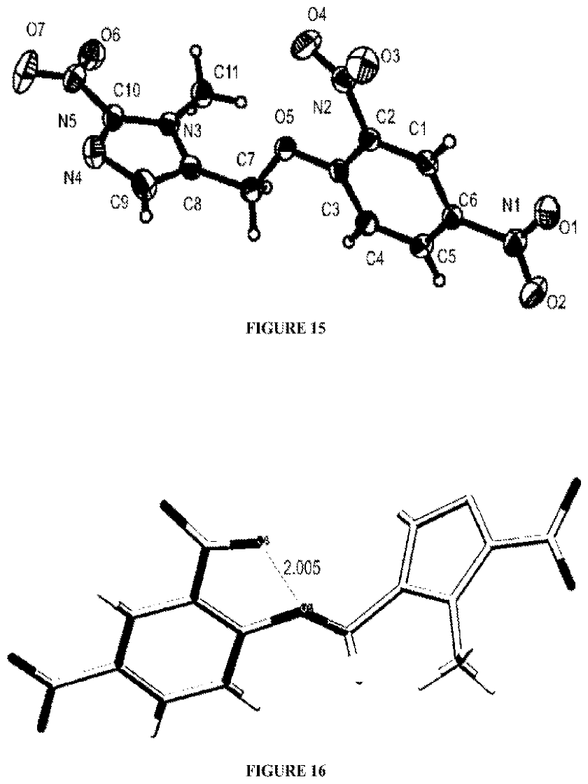

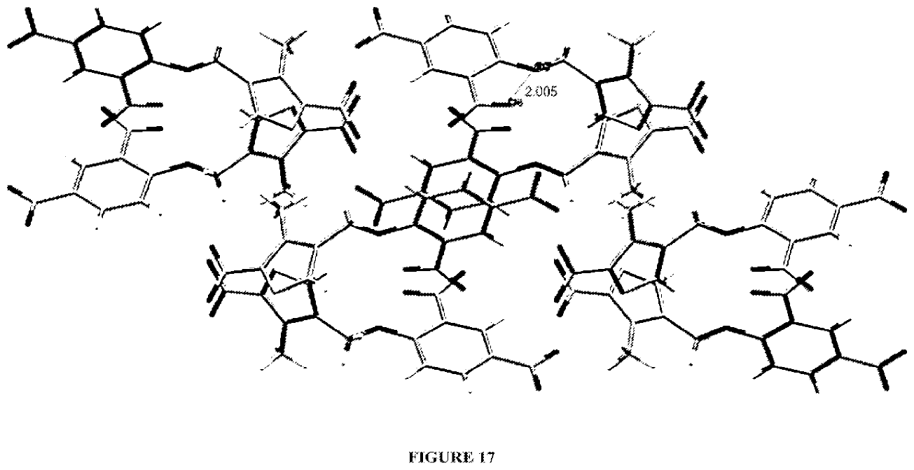

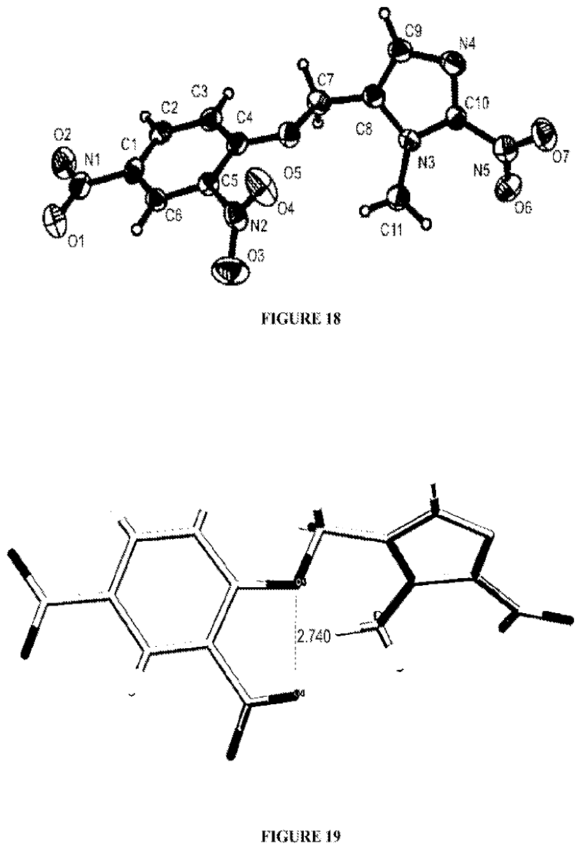

Crystal structure of Form A was determined at 298(2) K. Based on single crystal data, the single crystal is crystallized in orthorhombic system, Pbca space group with Rᵢₙₜ=6.0%, the final R1=[I>2σ(I)]=4.9% at 298(2)K. The Ortep image of Form A molecule is shown in FIG. 15. The asymmetric unit of Form A is shown in FIG. 16. The 3D packing image of Form A is shown in FIG. 17. Table 18 is the crystal dimension data of Form A

TABLE 18
Crystal dimension data of Form A.

| C₁₁H₉N₅O₇ | | F(000) = | 1328 |
|---|---|---|---|
| Mᵣ = | 323.23 | Dₓ = | 1.639 Mg m⁻³ |
| Orthorhombic, Pbca | | Mo Kα radiation, λ = | 0.71073 Å |
| a = | 12.4201 (5) Å | Cell parameters from | |
| | | 2354 reflections | |
| b = | 8.7368 (3) Å | θ = | 2.4-24.4° |
| c = | 24.1414 (10) Å | μ = | 0.14 mm⁻¹ |
| V = | 2619.63 (18) Å³ | T = | 298K |
| Z = | 8 | | |

Single crystal Form B suitable for single crystal analysis were picked from the crystalline Form B as shown in FIG. 2 shows XRPD pattern of Form B.

Crystal structure of Form B was determined at 298(2) K. Based on single crystal data, the single crystal is crystallized in monoclinic system, P2₁/c space group with Rᵢₙₜ=12.7%, the final R1=[J>26(I)]=7.7% at 298(2)K. The Ortep image of Form B molecule is shown in FIG. 20. The asymmetric unit of Form B is shown in FIG. 19. The 3D packing image of Form B is shown in FIG. 20. Table 19 is the crystal dimension data of Form B.

TABLE 19
Crystal dimension data of Form B.

| C₁₁H₉N₅O₇ | | F(000) = | 664 |
|---|---|---|---|
| Mᵣ = | 323.23 | Dₓ = | 1.577 Mg m⁻³ |
| Monoclinic, P2₁/c | | Mo Kα radiation, λ = | 0.71073 Å |
| a = | 10.3276 (15) Å | Cell parameters from | |
| | | 578 reflections | |
| b = | 18.141 (2) Å | θ = | 2.3-26.2° |
| c = | 7.5401 (9) Å | μ = | 0.13 mm⁻¹ |
| β = | 105.448 (4)° | T = | 298K |

| | | |
|---|---|---|
| V = | 1361.6 (3) Å³ | Needle, colourless |
| Z = | 4 | 0.08 0.04 × 0.2 mm |

### Example 9. Solubility Enhancing Excipient Screen of Compound (I)—Crystalline v. Amorphous Form

We evaluated solubility enhancement of crystalline Compound (I) Form B and Compound (I) in an amorphous form, as present in a spray dried dispersion formulation in suspension vehicles with solubility enhancing excipients.

In this experiment, amorphous drug/polymer colloids of Compound (I) were generated by spray dried dispersion (SDD) with Compound (I) and hydroxypropyl methylcellulose acetate succinate (HPMCAS-M). These experiments aimed to find a solubilizing agent for Compound (I), and TPGS was chosen as the excipient in dog studies. The extended dissolution time of Compound (I) prevented rapid uptake of Compound (I) in the plasma, and avoided the risk of a high Cmax (maximal blood plasma concentration). A high blood plasma concentration, or rather a rapid increase of that concentration, confers DNP toxicity.

SDD of the present screen has the following features:
Spray dried 120 g 25% Compound (I): HPMCAS-M.
   Spray solvent: 2.5% H₂O:Acetone
   Total solids: 6 wt % [1.5 wt % Compound (I), 4.5 wt % HPMCAS-M]
   87% yield
   PXRD was consistent with amorphous form
   Single Tg observed by mDSC at 75 °C

TABLE 20
Solubilization

| | Crystalline | | Amorphous | |
|---|---|---|---|---|
| | Compound (I) | | Compound (I) | |
| Solubilizing | Compound (I) Concentration (μg/mL) | | | |
| Excipient | t = 1 h | t = 7 h | t = 1 h | t = 7 h |
| TPGS 12.5 wt % | 190 | 270 | 964 | 608 |
| Phosal 50PG, | 11 | 11 | 34 | 14 |
| 0.5 wt % in water | | | | |
| Control (none) | 3.1 | 4.2 | 16 | 6.4 |
| HPBCD, 10 wt % | 3.0 | 5.6 | 18 | 3.8 |
| BCD, 2 wt % | 2.1 | 6.1 | 9.7 | 2.9 |
| Captisol, 6 wt % | 1.4 | 0.6 | 2.3 | 1.4 |
| EtOH, 7.5 vol % | 1.1 | 1.6 | 5.4 | 6.2 |
| PEG-400, 5 vol % | 0.7 | 2.6 | 3.5 | 7.0 |

*Control = 0.5 wt % MC with 0.1% SLS.

As shown in Table 20, there is a 4- to 5-fold difference in dissolution at 1 hour between crystalline and amorphous forms of Compound (I). There is also a difference in dissolution over time. As Table 20 shows, amorphous Compound (I) rapidly goes into solution, and then falls out, indicated by the lower numbers after prolonged incubation. The concentration of Crystalline Form B instead steadily increases in the media. Compound (I) is a class 2 compound with low solubility and high permeability, i.e. the dissolution is critical to achieving a slow increase of Compound (I) in plasma, and a highly solubilized form and/or formulation would lead to rapid up-take. If the dissolution increases (as it does with the amorphous form), the PK will spike and potentially reach unsafe plasma concentrations. As shown in Table 21, this is indeed the case.

As shown in Table 21, when Compound (I) is in an amorphous form, the free drug solubility increases by approximately 3-fold. Compound (I) in an amorphous form and TPGS in suspension have a combined effect on free drug solubility, resulting in an increase in free drug solubility by approximately 9-fold. Addition of TPGS to a suspension of crystalline Compound (I) increases free drug solubility by approximately 3-fold.

This result highlights the unpredicted results achieved in Examples 10 and 11, with micronized crystalline Form B, i.e. achieving a much higher bioavailability than expected.

TABLE 21
Rank Ordered by Free Drug Solubility Enhancement (Cmax)

| | | C-max | Concentration | AUC |
|---|---|---|---|---|
| Sample (Suspension = 20 mg/mL) | AUC | (μg/mL) | Enhancement | Enhancement |
| Amorphous Compound (I) in TPGS | 11150 | 45 | 8.7 | 11 |
| Amorphous Compound (I) | 5025 | 15 | 2.9 | 4.8 |
| in 0.5 wt % MC, 0.1 wt % SLS | | | | |
| Crystalline Compound (I) in TPGS | 3375 | 13 | 2.6 | 3.2 |
| Crystalline Compound (I) | 1047 | 5 | 1.0 | 1.0 |
| in 0.5 wt % MC, 0.1 wt % SLS | | | | |

All suspensions prepared at 20 mg/mL Compound (I).
The suspensions had suitable syringeability and colloidal stability for use in pre-clinical and clinical studies.
The excipient concentrations were determined based on maximum tolerable doses in dogs.

### Example 10. A Single Dose Oral Comparative Pharmacokinetics Studies of Micronized Compound (I) in Beagle Dogs

Crystalline Form B of Compound (I) (micronized and unmicronized) was administered to groups of dogs on a single occasion by oral capsule, as described in Table 22 below.

Crystalline Form B was sieved through a 40 mesh screen and micronized via jet milling. The injector and grinder gas pressures were optimized to 5.0 bar each to achieve a D90 of milled material of no more than 30 am.

TABLE 22
Group Designation and Administration.

| Dose | | Dose Level | Number of |
|---|---|---|---|
| Group | Compound (I) Description | (mg/kg) | Males |
| 1 | Unmicronized | 30 | 3 |
| 2 | Micronized Sample 1 | 30 | 3 |
| 3 | Micronized Sample 2 | 30 | 3 |
| | (PSD (D50) 1.8 μm*) | | |
| 4 | Micronized Sample 3 | 30 | 3 |
| | (PSD (D50) 12 μm*) | | |
| 5 | Micronized Sample 4 | 30 | 3 |
| | (PSD (D50) 1.2 μm*) | | |

*Reported particle size is D50.

A series of 8 blood samples (approximately 1 mL each) were collected from each dog at the following time-points relative to dosing on Day 1: 1, 2, 4, 6, 8, 12, 16, and 24 hours post-dose. All animals were returned to the ITR spare colony following collection of the last blood sample on Day 2.

For this purpose, each dog was bled by venipuncture and the samples were collected into tubes containing the anticoagulant, K₂EDTA. Following collection, the samples were centrifuged (2500 rpm for 10 minutes at approximately 4 °C) and the resulting plasma was recovered, divided into two aliquots (Set A and Set B), and stored frozen (<−60 °C) in appropriately labeled vials or tubes.

FIG. 21 shows the plasma concentration of 2,4-dinitrophenol after administration of micronized and non-micronized Compound (I).

The particle distribution size of Compound (I), mean Cₘₐₓ and mean AUC₂₄ₕ after administration are summarized in Table 23 below.

TABLE 23
Single Dose Oral Comparative Pharmacokinetics Study.

| | | | | | Mean |
|---|---|---|---|---|---|
| | | | | Mean Cₘₐₓ, | AUC₂₄ₕ , |
| Dose | D10 | D50 | D90 | ng/ml | ng * h/mL |
| Group | (μm) | (μm) | (μm) | (±SD) | (±SD) |
| 1 | 4.4 | 42 | 86 | 671 ± 182 | 9490 ± 4960 |
| 2 | 1.0 | 3.5 | 8.5 | 3730 ± 1797 | 40,539 ± 24,953 |
| 3 | 0.72 | 1.8 | 4 | 7737 ± 5777 | 81,321 ± 61,107 |
| 4 | 1.2 | 12 | 53 | 3620 ± 617 | 47,037 ± 9053 |
| 5 | 0.58 | 1.2 | 2 | 11,397 ± 7577 | 143,931 ± 89,716 |

* Micronized Sample 1 (Dose Group 2) is the identical batch that was used in Example 11 herein. Compound (I) was administered in capsules without any additional excipients.

### Example 11. Pharmacokinetics of Micronized Compound (I)

### Drug Administration

Crystalline Compound (I) or matching placebo was administered orally as a single dose. All subjects except the fed cohort were dosed in the morning after an 8-hour fast and remained in a semi-reclined position for 1 hour and fasting for 4 hours post-administration. Capsules were swallowed with 240 mL (8 fluid ounces) of room temperature water.

Micronized Compound (I), in the fasted state, was dosed at 600 mg, 1050 mg and 1400 mg. Compound (I) was absorbed rapidly with a median Tₘₐₓ from 1.50 to 1.75 hours and a median T_{lag} of 0.25 hours across all dose levels. The mean t_{1/2} was short and ranged from 1.12 hours to 1.73 hours across the dose cohorts. Mean apparent clearance and volume of distribution remained similar with increasing dose. Exposures of Compound (I), based on Cₘₐₓ and AUC, appear to be less than dose proportional for Cₘₐₓ (31=0.65) and only slightly less than for AUC_{inf} (31=0.96) across the dose range of 600 mg to 1400 mg.

2,4-Dinitrophenol appeared quickly after Compound (I) administration with a median T_{lag} of 0.25 hours and a median Tₘₐₓ ranging from 6.0 hours to 8.0 hours. The mean t_{1/2} was relatively long and ranged from 26.8 hours to 34.6 hours across all dose levels. Mean apparent clearance and volume of distribution remained similar with increasing dose. Exposures of 2,4-dinitrophenol, based on Cₘₐₓ and AUC, appear to be less than dose proportional for Cₘₐₓ and AUC_{inf} with slopes of 0.89 and 0.81, respectively across the dose range of 600 mg to 1400 mg.

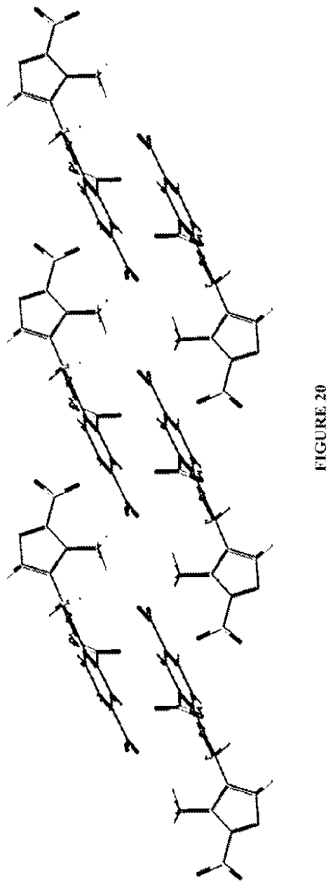

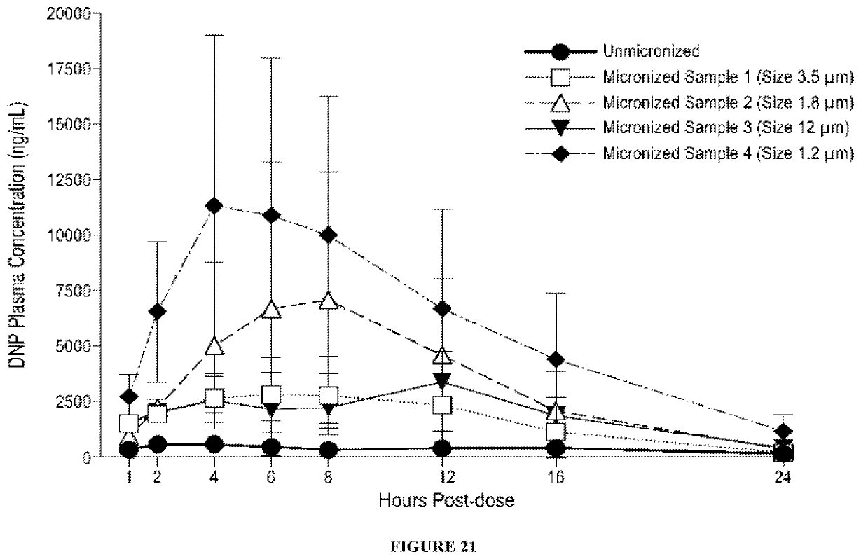

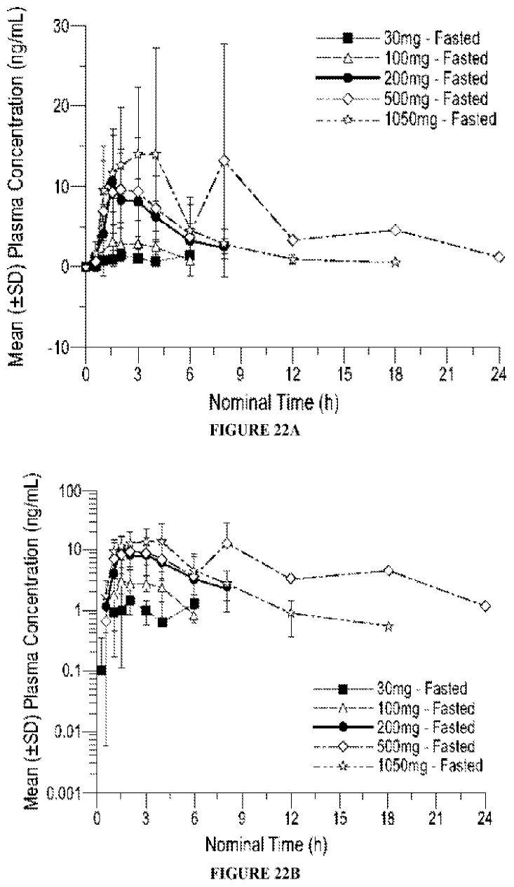

FIG. 22A and FIG. 22B show the mean (±SD) plasma Compound (I) concentration—time plot by dose following non-micronized Compound (I) oral administration (Linear (22A) and Semi-log Scale (22B)).

FIG. 23A and FIG. 23B show the mean (±SD) plasma 2,4-dinitrophenol concentration—time plot by dose following non-micronized Compound (I) oral administration (Linear (23A) and Semi-log Scale (23B)).

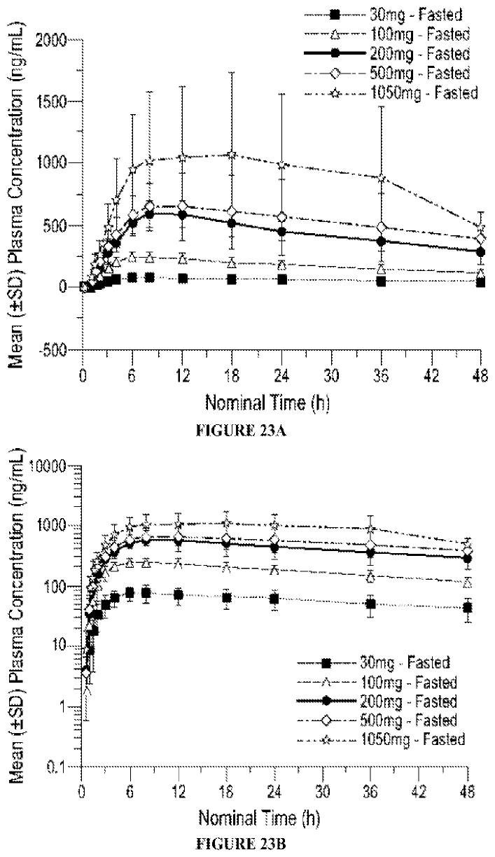

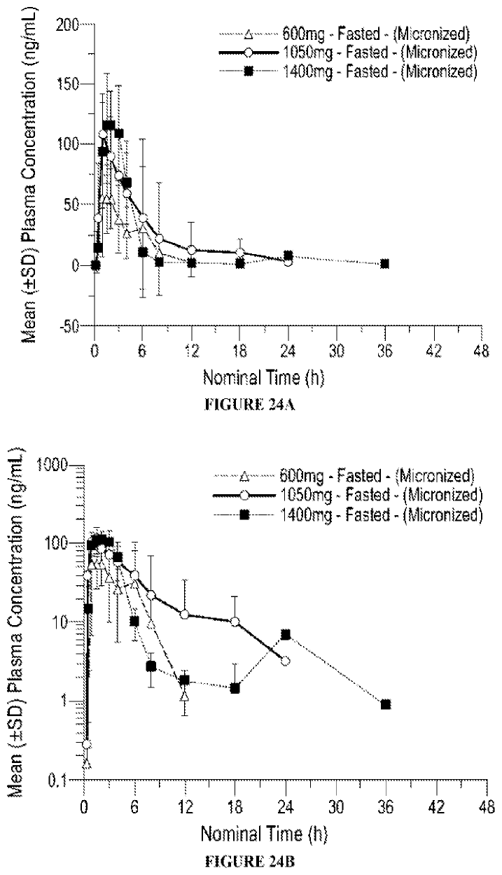

FIG. 24A and FIG. 24B show the mean (±SD) plasma Compound (I) concentration—time plot by dose following micronized Compound (I) oral administration (Linear and Semi-log Scale).

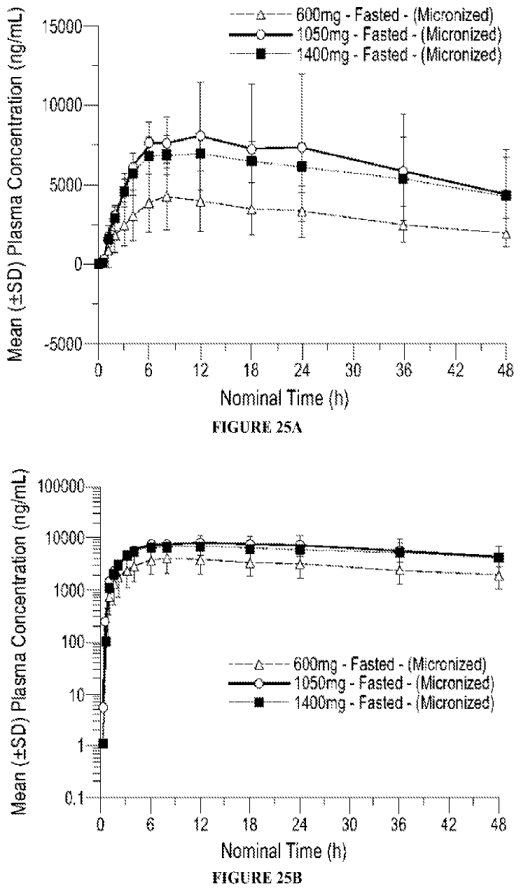

FIG. 25A and FIG. 25B below show the mean (±SD) plasma 2,4-dinitrophenol (DNP) concentration—time plot by dose following micronized Compound (I) oral administration (Linear and Semi-log Scale). DNP appeared quickly after a single dose of the micronized formulation of Compound (N), with a median T_{lag} of 0.25 hours and a median Tₘₐₓ ranging from 6.0 hours to 8.0 hours. The meant t_{1/2} was relatively long and ranged from 26.8 to 34.6 hours across dose levels. Mean apparent clearance and volume of distribution remained similar with increasing dose. Exposures of DNP were less than dose proportional across the dose range of 600 to 1400 mg of micronized Compound (I), with slopes of 0.89 and 0.81 for Cmax and AUCinf, respectively.

Table 24A shows Compound (I) Pharmacokinetic parameters after a single dose of non-micronized Compound (I). Table 24B shows Compound (I) pharmacokinetic parameters after a single dose of micronized crystalline Compound (I).

TABLE 24A
Mean (SD) Compound (I) Pharmacokinetic Parameters After a
Single Oral Dose of Non-micronized Compound (I) (Fasted Conditions)

| | Compound (I) Dose Level (Non-micronized Formulation, Fasted Treatments) | | | | |
|---|---|---|---|---|---|
| Pharmacokinetic | 30 mg | 100 mg | 200 mg | 500 mg | 1050 mg |
| Parameter | (N = 4) | (N = 6) | (N = 6) | (N = 8) | (N = 6) |
| AUCₗₐₛₜ | 4.53 | 10.7 | 38.3 | 53.8 | 69.1 |
| (h · ng/mL) | (0.913) | (4.27) | (12.8) | (44.1) | (41.4) |
| AUC₀₋₂₄ | NE | NE | NE | 68.0 | 71.7 |
| (h · ng/ml) | | | | (60.4)^{b} | (41.5) |
| AUC_{inf} | NE | NE | NE | NE | 72.0 |
| (h · ng/mL) | | | | | (41.2) |
| Cₘₐₓ (ng/mL) | 1.86 | 3.73 | 12.3 | 16.5 | 17.1 |
| | (0.656) | (1.52) | (5.29) | (7.81) | (12.0) |
| T_{lag} (h)^{a} | 0.500 | 0.500 | 0.500 | 0.250 | 0.250 |
| | (0.00, 0.517) | (0.500, 2.00) | (0.250, 0.500) | (0.250, 0.500) | (0.250, 0.500) |
| Tₘₐₓ (h)^{a} | 1.76 | 1.75 | 1.75 | 3.00 | 2.50 |
| | (1.00, 2.00) | (1.50, 3.00) | (1.75, 4.00) | (1.02, 8.00) | (1.00, 4.00) |
| t_{1/2} (h) | NE | NE | NE | NE | 2.32 |
| | | | | | (1.81) |
| CL/F (L/h) | NE | NE | NE | NE | 18 400 |
| | | | | | (8710) |
| V_{z}/F (L) | NE | NE | NE | NE | 62 900 |
| | | | | | (59500) |
| Cₘₐₓ /Dose | 0.0618 | 0.0373 | 0.0615 | 0.0329 | 0.0163 |
| (ng/ml/mg) | (0.0219) | (0.0152) | (0.0264) | (0.0156) | (0.0115) |
| AUCₗₐₛₜ/Dose | 0.151 | 0.107 | 0.191 | 0.108 | 0.0658 |
| (h · ng/mL/mg) | (0.0304) | (0.0427) | (0.0642) | (0.0882) | (0.0394) |
| AUC_{inf}/Dose | NE | NE | NE | NE | 0.0686 |
| (h · ng/ml/mg) | | | | | (0.0392) |

N = number of subjects in pharmacokinetic population;
n = number of subjects with non-missing values;
NE = not estimable, values were reported for fewer than 50% of the subjects.
^{a}Median (minimum. maximum)
^{b}n = 4.

TABLE 24B
Mean (SD) Compound (I) Pharmacokinetic Parameters After a
Single Oral Dose of micronized Crystalline Compound (I) (Fasted Conditions)

| Compound (I) | Crystalline Compound (I) Micronized Formulation (Fasted Conditions) | | | | | |
|---|---|---|---|---|---|---|
| Pharmacokinetic | 600 mg | | 1050 mg | | 1400 mg | |
| Parameter | (N = 6) | | (N = 6) | | (N = 6) | |
| AUCₗₐₛₜ | 246 | (117) | 529 | (445) | 433 | (110) |
| (h · ng/ml) | | | | | | |
| AUC₀₋₂₄ | 215 | (91.9)^{b} | 531 | (443) | 429 | (108) |
| (h · ng/ml) | | | | | | |
| AUC_{inf} | 215 | (91.9)^{b} | 534 | (449) | 428 | (121)^{b} |
| (h · ng/ml) | | | | | | |
| Cₘₐₓ (ng/ml) | 90.0 | (46.2) | 131 | (26.7) | 140 | (31.5) |
| T_{lag} (h)^{a} | 0.250 | (0.00, 0.267) | 0.250 | (0.00, 0.250) | 0.250 | (0.250, 0.250) |
| Tₘₐₓ (h)^{a} | 1.75 | (1.00, 6.00) | 1.75 | (1.00, 6.02) | 1.50 | (1.00, 3.00) |
| t_{1/2} (h) | 1.12 | (0.487)^{b} | 1.71 | (0.960) | 1.73 | (1.16)^{b} |
| CL/F (L/h) | 3360 | (1790)^{b} | 2710 | (1150) | 3480 | (909)^{b} |
| V_{z}/F (L) | 5540 | (3670)^{b} | 5640 | (2610) | 7720 | (3630)^{b} |
| Cₘₐₓ/Dose | 0.150 | (0.0770) | 0.125 | (0.0254) | 0.0998 | (0.0225) |
| (ng/ml/mg) | | | | | | |
| AUCₗₐₛₜ/Dose | 0.410 | (0.194) | 0.504 | (0.423) | 0.310 | (0.0782) |
| AUC_{inf}/Dose | 0.358 | (0.153)^{b} | 0.509 | (0.428) | 0.306 | (0.0863)^{b} |

N = number of subjects in pharmacokinetic population;
n = number of subjects with non-missing values;
NE = not estimable, values were reported for fewer than 50% of the subjects.
^{a}Median (minimum. maximum)
^{b}n = 4.

As shown in Table 24B, administration of the micronized formulation of crystalline Compound (I) 1050 mg resulted in an 8.8-fold increase in Compound (I) Cmax and a 6.9- to 7.2-fold increase in Compound (I) AUC relative to the non-micronized formulation of Compound (I) 1050 mg. Compound (I) Cₘₐₓ appeared to increase in a less than dose proportional manner and AUCinf increased in a slightly less than dose proportional manner (slope=0.96) across the range of 600 to 1400 mg of micronized crystalline Compound MI.

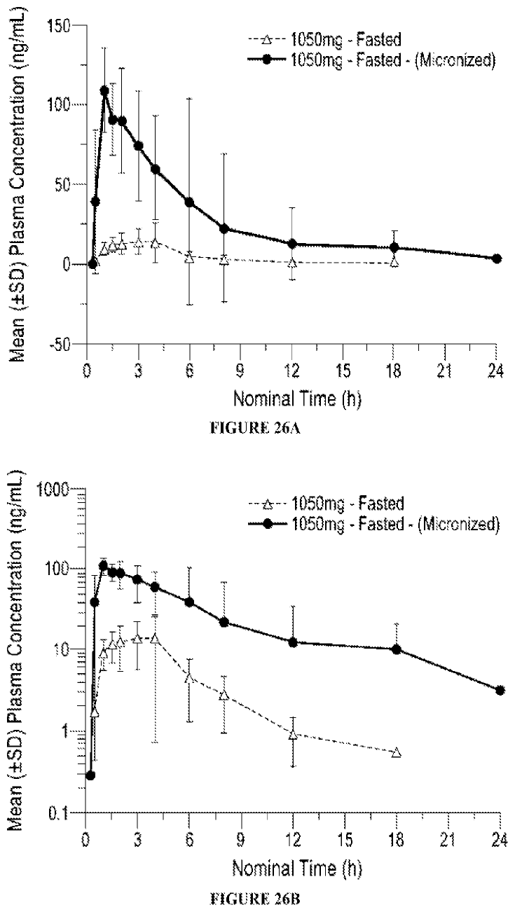

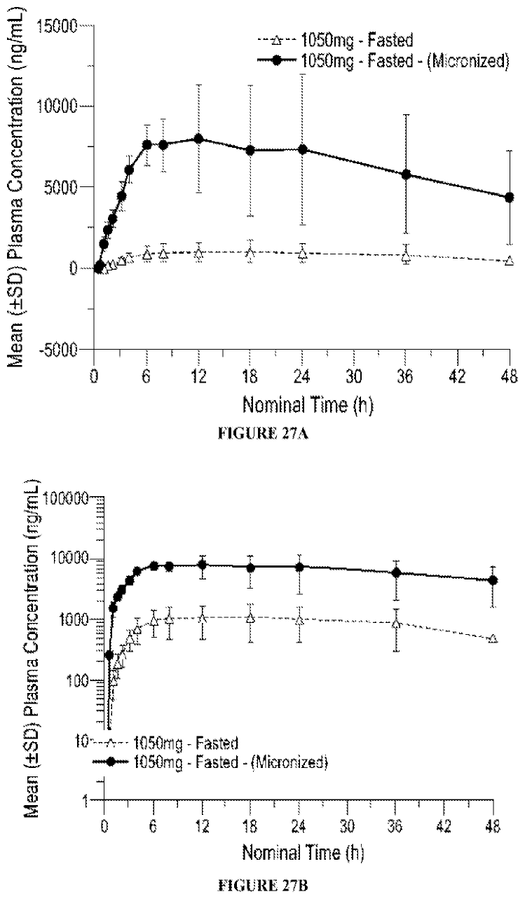

FIG. 26A and FIG. 26B compares the plasma Compound (I) concentration following 1050 mg micronized and non-micronized Compound (I) oral administration (Linear and Semi-log Scale). FIGS. 27A and 27B compares the plasma 2,4-dinitrophenol concentration following 1050 mg micronized and non-micronized Compound (I) oral administration (Linear and Semi-log Scale).

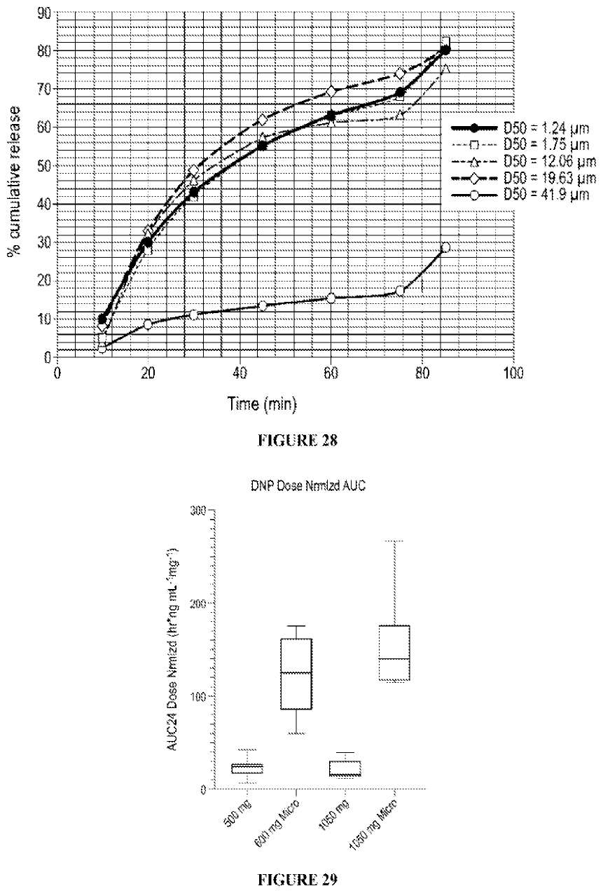

FIG. 28 shows the effect of particle size distribution of Compound (I) in cumulative release.

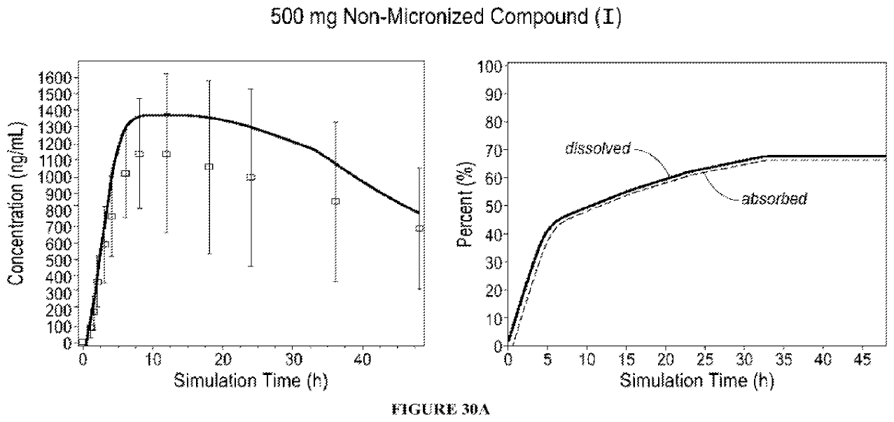

FIG. 29 compares the AUC of micronized and non-micronized Compound (I). It also shows increased exposure if Compound (I) is micronized.

The data demonstrates that micronized Compound (I) was absorbed faster and reached a higher plasma concentration compared to non-micronized Compound (I). The micronization of Compound (I) and the long half-life (i.e. build over time) provide unexpected results of therapeutically effective level for treating a wide range of diseases and conditions.

A positive food effect on Compound (I) absorption was evident as was the impact of Compound (I) particle size on absorption as two formulations of Compound (I) of different particle size were evaluated. At high single doses, there was evidence of saturation of absorption. The AUC/Cₘₐₓ ratio was approximately 18 regardless of dose.

The study demonstrates that
Compound (I) pharmacokinetics are characterized by rapid absorption and a fast elimination.
2,4-Dinitrophenol appears quickly and has a relatively slow elimination.
Micronization of Compound (I) increases Compound (I) and 2,4-dinitrophenol Cmax by >8.2-fold and increases Compound (I) and 2,4-dinitrophenol AUC by >6.90-fold.
Exposures of Compound (I) and 2,4-dinitrophenol following administration of micronized Compound (I), in general, increase in a less than dose proportional manner.

### Example 12. Pharmacokinetics of Micronized Compound (I)

A physiologically-based pharmacokinetic (PBPK) analysis was developed to establish a relationship between particle size and exposure. Micronization of crystalline Compound (I) improved its fraction absorbed, where a 600 mg micronized dose had the same fraction absorbed as a 30 mg non-micronized dose. Not only were plasma exposures of Compound (I) and one of its metabolites, DNP, much higher when dosing micronized crystalline Compound (I), but there were indications that liver metabolism was reduced as well, likely due to saturation of clearance. The effect of different particle size distributions on exposure was simulated, showing very little differences in exposure as long as particle sizes were small.

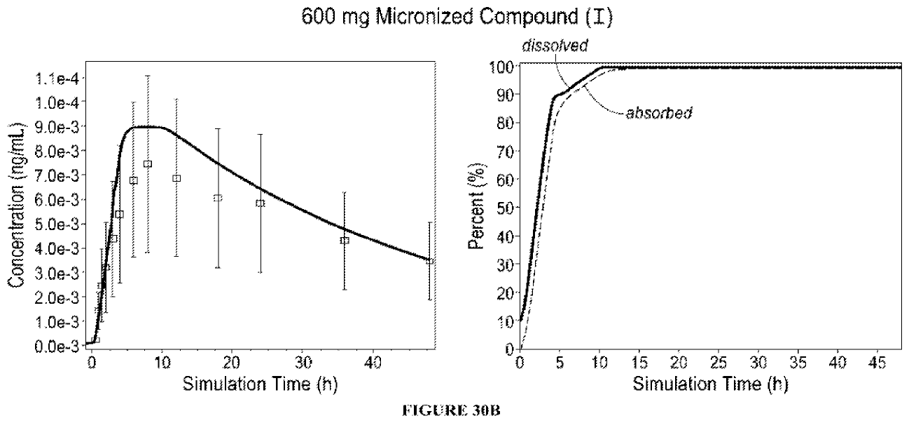

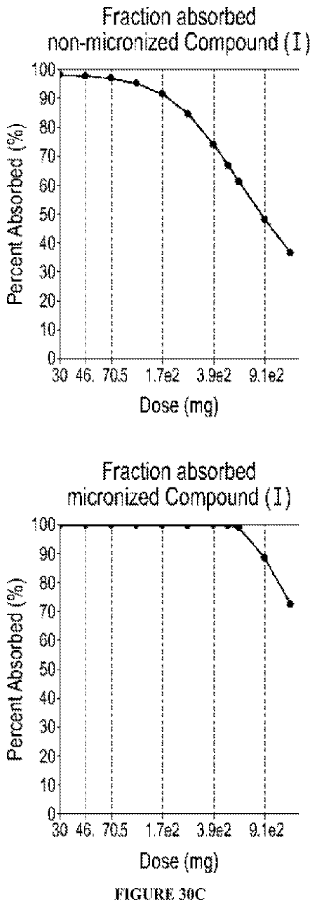

To illustrate the effect of particle size reduction, the outputs of the PBPK models for the 500 mg non-micronized dose and the 600 mg micronized dose are shown in FIGS. 30A-30C.

According to the model, the non-micronized dose resulted in 66% fraction absorbed (FIG. 30A), while the micronized dose dissolved almost completely with 98% fraction absorbed (FIG. 30B). The correlation between fraction absorbed and absorption for the dose range 30-1400 mg for the non-micronized and micronized doses are shown in FIG. 30C. A 30 mg non-micronized dose had the same fraction absorbed (Fa) as a 600 mg micronized dose, i.e. 98% (Table 25).

TABLE 25
Fraction Absorbed

| Dose (mg) | Compound (I) | Fₐ (%) |
|---|---|---|
| 30 | Non-micronized | 98 |
| 100 | Non-micronized | 95 |
| 200 | Non-micronized | 89 |
| 500 | Non-micronized | 66 |
| 1050 | Non-micronized | 44 |
| 600 | Micronized | 98 |
| 1050 | Micronized | 84 |
| 1400 | Micronized | 72 |

The PBPK model was also used to simulate the effect on exposure for hypothetical batches consisting of variable particle size distributions (Table 26). For comparison, both non-micronized as well as the micronized batch from Example 11 were added. The exposures of all micronized batches was comparable.

TABLE 26
Hypothetical Samples with different PSDs

| Hypothetical | | | |
|---|---|---|---|
| Sample | D10 (μm) | D50 (μm) | D90 (μm) |
| 1 | 3 | 7 | 15 |
| 2 | 0.9 | 3 | 11 |
| 3 | 1 | 5 | 13 |

Micronization had at least two observed effects on the crystalline Compound (I)+DNP exposure, relative to non-micronized Compound (I):
A significantly higher fraction absorbed, due to more dissolution of Compound (I).
A lower clearance, likely due to the higher liver exposure of Compound (I)+DNP saturating liver clearance.

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

We claim:

1. A crystalline form of 5-[(2,4-dinitrophenoxy)methyl]-1-methyl-2-nitro-1H-imidazole having the structure:

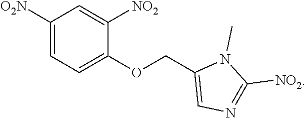 [SMILES: Cn1c(COc2ccc([N+](=O)[O-])cc2[N+](=O)[O-])cnc1[N+](=O)[O-]]

2. The crystalline form of claim 1, wherein the crystalline form is characterized by an X-ray powder diffraction pattern having characteristic peaks expressed, in degree 2θ, at approximately 13.3±0.2, 23.8±0.2, and 26.3±0.2.

3. The crystalline form of claim 1, wherein the crystalline form is characterized by an X-ray powder diffraction pattern having characteristic peaks expressed, in degree 2θ, at 9.8±0.2, 21.6±0.2, 27.3±0.2, and 28.1±0.2.

4. The crystalline form of claim 1, wherein the crystalline form has an X-ray powder diffraction pattern comprising four or more peaks, in 2-theta values, wherein the four or more peaks are selected from 9.8±0.2, 13.3±0.2, 21.6±0.2, 23.8±0.2, 26.3±0.2, 27.3±0.2, and 28.1±0.2.

5. The crystalline form of claim 1, wherein the crystalline form is characterized by an X-ray powder diffraction pattern having characteristic peaks expressed, in degree 2θ, at approximately 14.0±0.2, 25.8±0.2, 26.6±0.2, and 31.1±0.2.

6. The crystalline form of claim 1, wherein the crystalline form has an X-ray powder diffraction pattern comprising six or more peaks, in 2-theta values, wherein the six or more peaks are selected from 9.8±0.2, 13.3±0.2, 14.0±0.2, 21.6±0.2, 23.8±10.2, 25.8±0.2, 26.3±0.2, 26.6±0.2, 27.3±0.2, 28.1±0.2, and 31.1±0.2.

7. The crystalline form of claim 1, wherein the crystalline form has an X-ray powder diffraction pattern comprising eight or more peaks, in 2-theta values, wherein the eight or more peaks are selected from 9.8±0.2, 13.3±0.2, 14.0±0.2, 21.6±0.2, 23.8±0.2, 25.8±0.2, 26.3±0.2, 26.6±0.2, 27.3±0.2, 28.1±0.2, and 31.1±0.2.

8. The crystalline form of claim 1, wherein the crystalline form is characterized by an X-ray powder diffraction pattern having characteristic peaks expressed, in degree 2θ, at 9.0±0.2, 21.2±0.2, 23.2±0.2, 31.8±0.2 and 33.0±0.2.

9. The crystalline form of claim 1, wherein the crystalline form has an X-ray powder diffraction pattern comprising ten or more peaks, in 2-theta values, wherein the ten or more peaks are selected from 9.8±0.2, 9.0±0.2, 13.3±0.2, 14.0±0.2, 21.6±0.2, 21.2±0.2, 23.2±0.2, 23.8±0.2, 25.8±0.2, 26.3±0.2, 26.6±0.2, 27.3±0.2, 28.1±0.2, 31.1±0.2, 31.8±0.2, and 33.0±0.2.

10. The crystalline form of claim 1, wherein the crystalline form is characterized by an X-ray powder diffraction pattern having characteristic peaks expressed, in degree 2θ, at 17.7±0.2, 19.1±0.2, 30.0±0.2, and 34.7±0.2.

11. The crystalline form of claim 1, wherein the crystalline form is characterized by an X-ray powder diffraction pattern substantially as shown in FIG. 2.

12. The crystalline form of claim 1, wherein the crystalline form has a differential scanning calorimetry thermogram (DSC) characterized by an initial endothermic transition at about 182.3 °C±3 and a peak temperature at about 184 °C±3.

13. The crystalline form of claim 1, wherein the crystalline form has a thermogravimetric analysis (TGA) characterized by about 0.584% of weight loss at 175 °C±3.

14. The crystalline form of claim 1, wherein the crystalline form is characterized by an X-ray powder diffraction pattern having characteristic peaks expressed, in degree 2θ, at approximately 17.6±0.2, 24.9±0.2, 26.0±0.2, and 30.0±0.2.

15. The crystalline form of claim 1, wherein the crystalline form is characterized by an X-ray powder diffraction pattern having characteristic peaks expressed, in degree 2θ, at 16.4±0.2, 17.9±0.2, and 20.7±0.2.

16. The crystalline form of claim 1, wherein the crystalline form has an X-ray powder diffraction pattern comprising four or more peaks, in 2-theta values, wherein the four or more peaks are selected from 16.4±0.2, 17.6±0.2, 17.9±0.2, 20.7±0.2, 24.9±0.2, 26.0±0.2, and 30.0±0.2.

17. The crystalline form of claim 1, wherein the crystalline form is characterized by at least three peaks in an X-ray powder diffraction pattern expressed, in degree 2θ, at approximately 13.0±0.2, 16.1±0.2, 20.4±0.2, and 24.3±0.2.

18. The crystalline form of claim 1, wherein the crystalline form has an X-ray powder diffraction pattern comprising six or more peaks, in 2-theta values, wherein the six or more peaks are selected from 13.0±0.2, 16.1±0.2, 16.4±0.2, 17.6±0.2, 17.9±0.2, 20.4±0.2, 20.7±0.2, 24.3±0.2, 24.9±0.2, 26.0±0.2, and 30.0±0.2.

19. The crystalline form of claim 1, wherein the crystalline form has an X-ray powder diffraction pattern comprising eight or more peaks, in 2-theta values, wherein the eight or more peaks are selected from 13.0±0.2, 16.1±0.2, 16.4±0.2, 17.6±0.2, 17.9±0.2, 20.4±0.2, 20.7±0.2, 24.3±0.2, 24.9±0.2, 26.0±0.2, and 30.0±0.2.

20. The crystalline form of claim 1, wherein the crystalline form is characterized by at least three peaks in an X-ray powder diffraction pattern expressed, in degree 2θ, at approximately 22.8±0.2, 26.2±0.2, 31.1±0.2, and 33.6±0.2.

21. The crystalline form of claim 1, wherein the crystalline form has an X-ray powder diffraction pattern comprising ten or more peaks, in 2-theta values, wherein the ten or more peaks are selected from 13.0±0.2, 16.1±0.2, 16.4±0.2, 17.6±0.2, 17.9±0.2, 20.4±0.2, 20.7±0.2, 22.8±0.2, 24.3±0.2, 24.9±0.2, 26.0±0.2, 26.2±0.2, 30.0±0.2, 31.1±0.2, and 33.6±0.2.

22. The crystalline form of claim 1, wherein the crystalline form has an X-ray powder diffraction pattern comprising twelve or more peaks, in 2-theta values, wherein the twelve or more peaks are selected from 13.0±0.2, 16.1±0.2, 16.4±0.2, 17.6±0.2, 17.9±0.2, 20.4±0.2, 20.7±0.2, 22.8±0.2, 24.3±0.2, 24.9±0.2, 26.0±0.2, 26.2±0.2, 30.0±0.2, 31.1±0.2, and 33.6±0.2.

23. The crystalline form of claim 1, wherein the crystalline form is characterized by at least three peaks in an X-ray powder diffraction pattern expressed, in degree 2θ, at approximately 21.7±0.2, 29.1±0.2, 29.6±0.2, 30.7±0.2, and 37.2±0.2.

24. The crystalline form of claim 1, wherein the crystalline form is characterized by at least three peaks in an X-ray powder diffraction pattern expressed, in degree 2θ, at approximately 16.6±0.2, 24.1±0.2, 25.5±0.2, and 28.8±0.2.

25. The crystalline form of claim 1, wherein the crystalline form is characterized by at least three peaks in an X-ray powder diffraction pattern expressed, in degree 2θ, at approximately 14.4±0.2, 19.0±0.2, 28.5±0.2, 35.7±0.2, 36.2±0.2, and 38.9±0.2.

26. The crystalline form of claim 1, wherein the crystalline form is characterized by an X-ray powder diffraction pattern substantially as shown in FIG. 1.

27. The crystalline form of claim 1, wherein the crystalline form has a differential scanning calorimetry thermogram (DSC) profile characterized by an endothermic transition at a temperature between 157 °C±3 and 162 °C±3 and a second endothermic transition at 183 °C±3.

28. The crystalline form of claim 1, wherein the crystalline form has a thermal gravimetric analysis (TGA) characterized by about 0.704% of weight loss at 175 °C±3.

29. The crystalline form of any one of claims 2-13, which is crystalline Form B.

30. The crystalline form of any one of claims 14-28, which is crystalline Form A.

31. A micronized crystalline form of 5-[(2,4-dinitrophenoxy)methyl]-1-methyl-2-nitro-1H-imidazole having the structure:

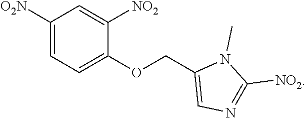 [SMILES: Cn1c(COc2ccc([N+](=O)[O-])cc2[N+](=O)[O-])cnc1[N+](=O)[O-]]

32. The micronized crystalline form of claim 31, which is micronized crystalline Form B.

33. The micronized crystalline form of claim 32, wherein micronized crystalline Form B is characterized by an X-ray powder diffraction pattern expressed, in degree 2θ, as described in any one of claims 2-11.

34. The micronized crystalline form of claim 31, which is micronized crystalline Form A.

35. The micronized crystalline form of claim 33, wherein micronized crystalline Form A is characterized by an X-ray powder diffraction pattern expressed, in degree 2θ, as described in any one of claims 14-28.

36. The micronized crystalline form of any one of claims 31-35, wherein the micronized crystalline form has a particle size distribution (D50) of about 1 μm to about 10 μm.

37. The micronized crystalline form of any one of claims 31-36, wherein the micronized crystalline form has a particle size distribution (D50) of about 1 μm to about 5 μm.

38. The micronized crystalline form of any one of claims 31-37, wherein the micronized crystalline form has a particle size distribution (D50) of about 2 μm to about 4 μm.

39. The micronized crystalline form of any one of claims 31-38, wherein the micronized crystalline form has a particle size distribution (D50) of about 3 μm.

40. The micronized crystalline form of any one of claims 31-39, wherein the micronized crystalline form has a particle size distribution (D90) of about 5 μm to about 15 μm.

41. The micronized crystalline form of any one of claims 31-39, wherein the micronized crystalline form has a particle size distribution (D90) of about 8 μm to about 13 μm.

42. The micronized crystalline form of any one of claims 31-39, wherein the micronized crystalline form has a particle size distribution (D90) of about 9 μm.

43. A pharmaceutical composition comprising the crystalline form of any one of claims 1-30 and a pharmaceutically acceptable carrier.

44. A pharmaceutical composition comprising the micronized crystalline form of any one of claims 31-42 and a pharmaceutically acceptable carrier.

45. A method of treating a mitochondria-related disorder or condition in a subject comprising administering to the subject in need thereof an effective amount of the crystalline form of any one of claims 1-30 or the micronized crystalline form of any one of claims 31-42.

46. The method of claim 45, wherein the disorder or condition is metabolic disorders, diabetes, or diabetes-associated complications.

47. The method of claim 45, wherein the disorder is obesity or excess body fat.

48. The method of claim 45, wherein the disorder or condition is metabolic disorders, diabetes, or diabetes-associated complications.

49. The method of claim 45, wherein the disorder or condition is type 2 diabetes (T2DM).

50. The method of claim 45, wherein the disorder is non-alcoholic fatty liver disease (NAFLD).

51. The method of claim 45, wherein the disorder is non-alcoholic steatohepatitis (NASH).

52. The method of claim 45, wherein the disorder is hepatic steatosis.

53. The method of claim 45, wherein the disorder is insulin resistance or intolerance.

54. The method of claim 45, wherein the disorder is dyslipidemia.

55. The method of claim 45, wherein the disorder is cardiovascular disease.

56. The method of claim 45, wherein the disorder is atherosclerosis.

57. A method of reducing adiposity, controlling or preventing of weight gain in a subject comprising administering to the subject in need thereof an effective amount of the crystalline form of any one of claims 1-30 or the micronized crystalline form of any one of claims 31-42.

58. A method for stimulating oxygen consumption rate (OCR) in a subject comprising administering to the subject in need thereof an effective amount of the crystalline form of any one of claims 1-30 or the micronized crystalline form of any one of claims 31-42.
